(19) United States
(12) Patent Application Publication
(10) Pub. No.: US 2026/0327203 A1
(43) Pub. Date: Oct. 22, 2026

(54) HAPH ANALOGS AND THERAPEUTIC AND DIAGNOSTIC USES THEREOF

(71) Applicant: MEDICAL RESEARCH & CONSULTING SERVICES, LLC, Encino, CA (US)

(72) Inventors: Thomas Lomis, Encino, CA (US); Jehangir Mistry, Encino, CA (US)

(21) Appl. No.: 19/111,996

(22) PCT Filed: Sep. 15, 2023
(86) PCT No.: PCT/US2023/074392

§ 371 Date: Mar. 14, 2025

Related U.S. Application Data
(60) Provisional application No. 63/407,507, filed on Sep. 16, 2022.
(60) Provisional application No. 63/422,340, filed on Nov. 3, 2022.

(51) Int. Cl. C07D 401/14; A61K 31/4439; A61K 51/04; A61P 35/00; C07B 59/00; C07F 1/08

(52) U.S. Cl. CPC C07D 401/14; A61K 31/4439; A61K 51/0455; A61P 35/00; C07B 59/002; C07F 1/08

(57) ABSTRACT

The present disclosure provides novel compounds and their metal chelates, for diagnostic, therapeutic and/or theranostic use in the treatment of cancer, tumor and other oxygen activity related diseases. The novel compounds are analogues of bleomycin (BLM).

### CROSS REFERENCES TO RELATED APPLICATIONS

The present PCT application claims the priority of U.S. Provisional Application USSN: 63/407,507, filed Sep. 16, 2022, and U.S. Provisional Application USSN: 63/422,340, filed Nov. 3, 2022, the entire disclosure of both of which is incorporated herein by reference.

### FIELD OF THE INVENTION

The present invention relates generally to compounds and their uses, such as in disease diagnosis and treatment.

### BACKGROUND OF THE INVENTION

Bleomycin (BLM) is an antibiotic glycopeptide that has been studied previously as an anti-tumor drug. BLM's analogs, such as N-(2-(imidazol-3-yl)ethyl)-6-(((2-(imidazol-3-yl)ethyl)-amino)methyl)-2-pyridinecarboxamide (hereinafter “HAPH”), have been synthesized and investigated. There is a need for developing more bleomycin analogs, such as HAPH analogs or derivatives, for medical use.

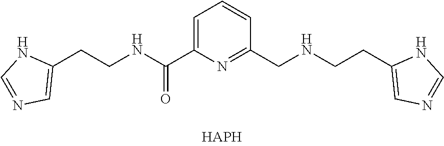 [SMILES: O=C(NCCc1cnc[nH]1)c1cccc(CNCCc2cnc[nH]2)n1]

### SUMMARY OF THE INVENTION

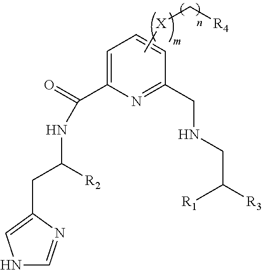

One aspect of the present disclosure encompasses a compound of Formula (I). In Formula (I), R₁ and R₃ each is independently selected from the substituted or unsubstituted group consisting of hydrogen, halogen, —NH₂, —C(O)NH₂, —C(O)OH, —C(O)OMe, —C(O)R₅, —C(O)NR₅, —OC(O)R₅, —C(O)OR₅, —(C₁-C₆ alkylene), —C₁-C₆ alkyl, —C₂-C₆ alkenyl, —C₂-C₆ alkynyl, —C₁-C₆ alkoxy, —C₁-C₆ heteroalkyl, —C₃-C₁₂ cycloalkyl, —C₆-C₁₄ aryl, —C₆-C₁₄ aryloxy, —C₆-C₁₄ aryl, 3-12 membered heterocyclyl, 3-12 membered heterocycloalkyl, 5-14 membered heteroaryl, —SH, —SMe, —SR₅, —S(O)₂R₅, —SR₅-aryl, and SR₅-aryl-O—R₅; and R₂ is selected from the substituted or unsubstituted group consisting of hydrogen, halogen, —C₁-C₆ alkyl, —C₂-C₆ alkenyl, and —C₂-C₆ alkynyl. In the Formula (I), the motif Xm-(CH₂)n-R₄ can be either at para- or meta-position relative to Nitrogen on the pyridine ring. X is —O—, —CH₂O—, —CH₂C(O)—, CH₂—NR₅—C(O)—, —NR₅—, —CH₂NR₅—, —CH₂S—, or —S—; and m is the number 0 or 1, and when m is 0, the methylene group is directly attached to the pyridine ring. Further, n is zero or an integer of 1, 2, 3, 4, 5, 6, 7, 8, 9, or 10; and R₄ is selected from the substituted or unsubstituted group consisting of hydrogen, hydroxyl, —NH₂, —C(O)NH₂, —NHC(O), —C(O)OH, —C(O)OMe, —C(O)R₅, —C(O)NR₅, —OC(O)R₅, —C(O)OR₅—, —N₃, —(CH₂)₁₀—N₃; —NH—C(O)—(CH₂)₁₁—N₃, —C₁-C₁₅ alkylazide, —C≡CH, —(CH₂)₁₀—C≡CH; —NH—C(O)—(CH₂)₁₁—C≡CH; —C₁-C₁₅—C≡CH; —SH, —SMe, —SR₅, —S(O)₂R₅, —SR₅-aryl, SR₅-aryl-O—R₅, —C₁-C₁₅ alkyl, —C₂-C₁₅ alkenyl, —C₂-C₁₅ alkynyl, and phosphate. The R₅ is selected from the group consisting of hydrogen, C₁-C₆ alkyl, C₂-C₆ alkenyl, and C₂-C₆ alkynyl.

In one aspect, Formula (I) expressly excludes those compounds that when m=n=0, R₁ is —NH₂, —SH, —SCH₃, SCH₂C₆H₄OCH₃, or 5-imidazolyl. In other words, Formula (I) may have a proviso clause, that when m=n=0, R₁ is not selected from the group consisting of —NH₂, —SH, —SCH₃, SCH₂C₆H₄OCH₃, and 5-imidazolyl.

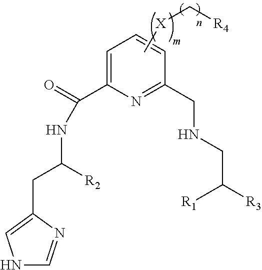

Another aspect of the present disclosure encompasses a compound of Formula (II), wherein the motif Xm-(CH₂)n-R₄ is at para-position relative to Nitrogen on the pyridine ring.

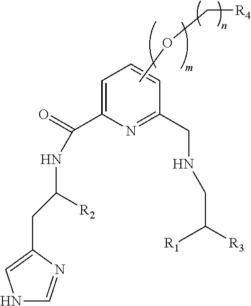

In yet another aspect, the compound is of Formula (III). In Formula (III), X is —O—; and m is the number 0 or 1. Further when m is 0, the methylene group is directly connected to the pyridine ring. In Formula (III), symbol “n” is zero or an integer of 1, 2, 3, 4, 5, 6, 7, 8, 9, 10. R₄ is selected from the substituted or unsubstituted group consisting of hydrogen, hydroxyl, —NH₂, —C(O)NH₂, —NHC(O), —C(O)OH, —C(O)OMe, —C(O)R₅, —C(O)NR₅, —OC(O)R₅, —C(O)OR₅—, —N₃, —(CH₂)₁₀—N₃; —NH—C(O)—(CH₂)₁₁—N₃, —C₁-C₁₅ alkylazide, —C≡CH, —(CH₂)₁₀—C≡CH; —NH—C(O)—(CH₂)₁₁—C≡CH; —C₁-C₁₅—C≡CH; —SH, —SMe, —SR₅, —S(O)₂R₅, —SR₅-aryl, SR₅-aryl-O—R₅, —C₁-C₁₅ alkyl, —C₂-C₁₅ alkenyl, —C₂-C₁₅ alkynyl, and phosphate. Each R₅ is selected independently from the group consisting of C₁-C₆ alkyl, C₂-C₆ alkenyl, and C₂-C₆ alkynyl. Other substituents are the same as the compound of Formula (I). In some aspect, Formula (III) has the proviso that m and no could not be both zero, in other words, Formula (III) may exclude those compounds that m=n=0.

In yet another aspect, the R₁ and R₃ of Formula (I) may together form an imidazole ring, which may optionally be further substituted. In yet another aspect, the compound of the present disclosure is of Formula (IV).

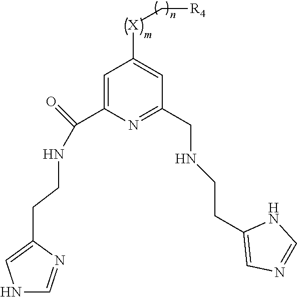

Another aspect of the present disclosure encompasses a compound corresponding to Formula (II) with the following substituents:

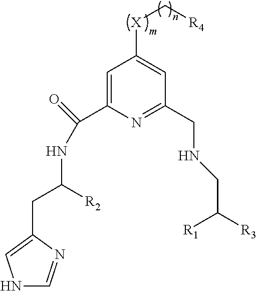

| NAME | R₁ | R₂ | R₃ | R₄ | X | m | n |
|---|---|---|---|---|---|---|---|
| HAPH-2 | 5-imidazolyl | H | H | NH₂ | N/A | 0 | 1 |
| HAPH-2 | 5-imidazolyl | H | H | NH₂ HCl | N/A | 0 | 1 |
| HCl Salt | | | | | | | |
| HAPH-2 | 5-imidazolyl | H | H | N₃ | CH₂—NHC(O) | 0 | 11 |
| Azide | | | | | | | |
| HAPH-2 | 5-imidazolyl | H | H | CΞC | CH₂—NHC(O) | 0 | 11 |
| Ethyne | | | | | | | |
| HAPH-2 | 5-imidazolyl | H | H | N₃ | N/A | 0 | 1 |
| Methylene | | | | | | | |
| Azide | | | | | | | |
| HAPH-3 | 5-imidazolyl | H | H | OH | N/A | 0 | 1 |
| HAPH-4 | 5-imidazolyl | H | H | COOH | N/A | 0 | 2 |
| HAPH-5 | 5-imidazolyl | H | H | COOH | N/A | 0 | 3 |
| HAPH-6 | 5-imidazolyl | H | H | NH₂ | N/A | 0 | 2 |
| HAPH-7 | 5-imidazolyl | H | H | NH₂ | N/A | 0 | 3 |
| HAPH-8 | 5-imidazolyl | H | H | OH | N/A | 0 | 2 |
| HAPH-9 | 5-imidazolyl | H | H | OH | N/A | 0 | 3 |
| HAPH-10 | 5-imidazolyl | H | H | NH₂ | O | 1 | 2 |
| HAPH-11 | 5-imidazolyl | H | H | COOH | O | 1 | 2 |
| HAPH-12 | 5-imidazolyl | H | H | OH | O | 1 | 2 |
| HAPH-13 | 5-imidazolyl | H | H | COOH | N/A | 0 | 1 |
| AMPHIS-1N | —NH₂ | —C(O)OCH₃ | H | NH₂ | N/A | 0 | 1 |
| AMPHIS-1O | —NH₂ | —C(O)OCH₃ | H | OH | N/A | 0 | 1 |
| AMPHIS-1A | —NH₂ | —C(O)OCH₃ | H | COOH | N/A | 0 | 1 |
| PYML-1N | NH₂ | —C(O)OH | —C(O)NH₂ | NH₂ | N/A | 0 | 1 |
| PYML-1O | NH₂ | —C(O)OH | —C(O)NH₂ | OH | N/A | 0 | 1 |
| PYML-1A | NH₂ | —C(O)OH | —C(O)NH₂ | COOH | N/A | 0 | 1 |
| SAPH-1N | —SMe | H | H | NH₂ | N/A | 0 | 1 |
| SAPH-1O | —SMe | H | H | OH | N/A | 0 | 1 |
| SAPH-1A | —SMe | H | H | COOH | N/A | 0 | 1 |
| SAPH-2N | —SCH₂C₆H₄Ome | H | H | NH₂ | N/A | 0 | 1 |
| SAPH-2O | —SCH₂C₆H₄Ome | H | H | OH | N/A | 0 | 1 |
| SAPH-2A | —SCH₂C₆H₄Ome | H | H | COOH | N/A | 0 | 1 |
| SAPH-3N | —SH | H | H | NH₂ | N/A | 0 | 1 |
| SAPH-3O | —SH | H | H | OH | N/A | 0 | 1 |
| SAPH-3A | —SH | H | H | COOH | N/A | 0 | 1 |

Another aspect of the present disclosure comprises a metal chelate of a compound of Formula (V), wherein the metal is selected from the group comprising of or consisting of Fe, Cu, Cr, Mg, Mn, Co, Ni, Zn, Ga, In, Y, and Ag;

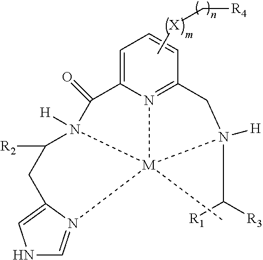

In Formula (V), R₁ and R₃ each is independently selected from the substituted or unsubstituted group consisting of hydrogen, halogen, —NH₂, —C(O)NH₂, —C(O)OH, —C(O)Ome, —C(O)R₅, —C(O)NR₅, —OC(O)R₅, —C(O)OR₅, —(C₁-C₆ alkylene), —C₁-C₆ alkyl, —C₂-C₆ alkenyl, —C₂-C₆ alkynyl, —C₁-C₆ alkoxy, —C₁-C₆ heteroalkyl, —C₃-C₁₂ cycloalkyl, —C₆-C₁₄ aryl, —C₆-C₁₄ aryloxy, —C₁₄ aryl, 3-12 membered heterocyclyl, 3-12 membered heterocycloalkyl, 5-14 membered heteroaryl, —SH, —SMe, —SR₅, —S(O)₂R₅, —SR₅-aryl, and SR₅-aryl-O—R₅. R₂ is selected from the substituted or unsubstituted group consisting of hydrogen, halogen, —C₁-C₆ alkyl, —C₂-C₆ alkenyl, and —C₂-C₆ alkynyl. In Formula (V), the motif Xm-(CH₂)n-R₄ can be either at para- or meta-position relative to Nitrogen on the pyridine ring. X is —O—, —CH₂NR₅—, CH₂O—, —CH₂C(O)—, CH₂—NR₅—C(O)—, —NR₅—, —CH₂S—, or —S—; and m is the number 0 or 1; and when m is 0, the methylene group is directly attached to the pyridine ring. Further, n is zero or an integer of 1, 2, 3, 4, 5, 6, 7, 8, 9, or 10; and R₄ is selected from the substituted or unsubstituted group consisting of hydrogen, hydroxyl, —NH₂, —C(O)NH₂, —NHC(O), —C(O)OH, —C(O)OMe, —C(O)R₅, —C(O)NR₅, —OC(O)R₅, —C(O)OR₅—, —N₃, —(CH₂)₁₀—N₃; —NH—C(O)—(CH₂)₁₁—N₃, —C₁-C₁₅ alkylazide, —C≡CH, —(CH₂)₁₀—C≡CH; —NH—C(O)—(CH₂)₁₁—C≡CH; —C₁-C₁₅—C≡CH; —SH, —SMe, —SR₅, —S(O)₂R₅, —SR₅-aryl, SR₅-aryl-O—R₅, —C₁-C₁₅ alkyl, —C₂-C₁₅ alkenyl, —C₂-C₁₅ alkynyl, and phosphate. Each R₅ is independently selected from the group consisting of C₁-C₆ alkyl, C₂-C₆ alkenyl, and C₂-C₆ alkynyl.

Another aspect of the present disclosure excludes the metal chelates of Formula (V), when metal is Fe or Cu, m=n=0, and R₁ is —SH, SMe, —SCH₂C₆H₄Ome or 5-imidazolyl. In other words, the present disclosure has the proviso clause that when metal is Fe or Cu, m=n=0, R₁ is —SH, SMe, —SCH₂C₆H₄Ome or 5-imidazolyl Formula (V), the metal chelates formed thereupon are NOT encompassed.

Another aspect of the present disclosure encompasses the metal chelate of Formula (V), and the metal is a metal ion comprises Fe (II), Fe (III), Cu (II), Cr (III), Cr (VI), Mg (II), Mn (II), Mn (III), Mn (VI), Co (II), Co (III), Ni (II), Zn (II), Sc (II), Ga (III), In (III), Y (III) and Ag (I). In another aspect, the metal is an isotope form or a radioisotope form. In another aspect, the metal chelate is a metal chelate of Formula (VI)

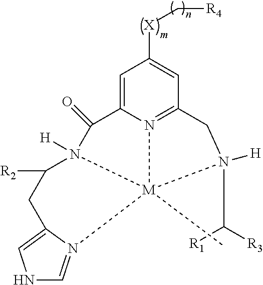

| NAME | R₁ | R₂ | R₃ | R₄ | X | m | n |
|---|---|---|---|---|---|---|---|
| AMPHIS | —NH₂ | —C(O)OCH₃ | H | H | N/A | 0 | 0 |
| PYML | NH₂ | —C(O)OH | —C(O)NH₂ | H | N/A | 0 | 0 |
| SAPH-1 | —SMe | H | H | H | N/A | 0 | 0 |
| SAPH-2 | —SCH₂C₆H₄OMe | H | H | H | N/A | 0 | 0 |
| SAPH-3 | —SH | H | H | H | N/A | 0 | 0 |
| HAPH-1 | 5-imidazolyl | H | H | H | N/A | 0 | 0 |
| HAPH-2 | 5-imidazolyl | H | H | NH₂ | N/A | 0 | 1 |
| HAPH-2 | 5-imidazolyl | H | H | NH₂ HCl | N/A | 0 | 1 |
| HCl Salt | | | | | | | |
| HAPH-2 | 5-imidazolyl | H | H | N₃ | CH₂—NHC(O) | 0 | 11 |
| Azide | | | | | | | |
| HAPH-2 | 5-imidazolyl | H | H | CΞC | CH₂—NHC(O) | 0 | 11 |
| Ethyne | | | | | | | |
| HAPH-2 | 5-imidazolyl | H | H | N₃ | N/A | 0 | 1 |
| Methylene | | | | | | | |
| Azide | | | | | | | |
| HAPH-3 | 5-imidazolyl | H | H | OH | N/A | 0 | 1 |
| HAPH-4 | 5-imidazolyl | H | H | COOH | N/A | 0 | 2 |
| HAPH-5 | 5-imidazolyl | H | H | COOH | N/A | 0 | 3 |
| HAPH-6 | 5-imidazolyl | H | H | NH₂ | N/A | 0 | 2 |
| HAPH-7 | 5-imidazolyl | H | H | NH₂ | N/A | 0 | 3 |
| HAPH-8 | 5-imidazolyl | H | H | OH | N/A | 0 | 2 |
| HAPH-9 | 5-imidazolyl | H | H | OH | N/A | 0 | 3 |
| HAPH-10 | 5-imidazolyl | H | H | NH₂ | O | 1 | 2 |
| HAPH-11 | 5-imidazolyl | H | H | COOH | O | 1 | 2 |
| HAPH-12 | 5-imidazolyl | H | H | OH | O | 1 | 2 |
| HAPH-13 | 5-imidazolyl | H | H | COOH | N/A | 0 | 1 |
| AMPHIS-1N | —NH₂ | —C(O)OCH₃ | H | NH₂ | N/A | 0 | 1 |
| AMPHIS-1O | —NH₂ | —C(O)OCH₃ | H | OH | N/A | 0 | 1 |
| AMPHIS-1A | —NH₂ | —C(O)OCH₃ | H | COOH | N/A | 0 | 1 |
| PYML-1N | NH₂ | —C(O)OH | —C(O)NH₂ | NH₂ | N/A | 0 | 1 |
| PYML-1O | NH₂ | —C(O)OH | —C(O)NH₂ | OH | N/A | 0 | 1 |
| PYML-1A | NH₂ | —C(O)OH | —C(O)NH₂ | COOH | N/A | 0 | 1 |
| SAPH-1N | —SMe | H | H | NH₂ | N/A | 0 | 1 |
| SAPH-1O | —SMe | H | H | OH | N/A | 0 | 1 |
| SAPH-1A | —SMe | H | H | COOH | N/A | 0 | 1 |
| SAPH-2N | —SCH₂C₆H₄Ome | H | H | NH₂ | N/A | 0 | 1 |
| SAPH-2O | —SCH₂C₆H₄Ome | H | H | OH | N/A | 0 | 1 |
| SAPH-2A | —SCH₂C₆H₄Ome | H | H | COOH | N/A | 0 | 1 |
| SAPH-3N | —SH | H | H | NH₂ | N/A | 0 | 1 |
| SAPH-3O | —SH | H | H | OH | N/A | 0 | 1 |
| SAPH-3A | —SH | H | H | COOH | N/A | 0 | 1 |

In yet another aspect, the present disclosure encompasses a composition comprising an amount of the aforementioned compounds, the metal chelates, with a carrier. In one aspect, the amount is a pharmaceutical effective amount, and the carrier is a pharmaceutically acceptable carrier. In yet another aspect, the amount is a diagnostic effective amount, and the carrier is a diagnostically acceptable carrier. In yet another aspect, the amount is a theranostic effective amount, and the carrier is a theranostic acceptable carrier. In yet another aspect, the carrier is suitable for parenteral delivery or for enteral delivery. In another aspect, the carrier is suitable for injection. In yet another aspect, the injection comprises intravenous injection, intratumor injection, subcutaneous injection, intramuscular injection, or intrathecal injection. In yet another aspect, the composition is in a unit dosage form.

In another aspect, the present disclosure encompasses a method of modulating oxygen activity, a method of inducing DNA scission, and/or a method inducing oxygen radical formulation. Such methods comprise administering to a subject in need thereof an effective amount of the aforementioned HAPH analogs, metal chelates, or the compositions thereof. The subject is having or suspected of having a cancer, a tumor, a blood pool, a thrombus, an inflammation, a hypoxia, a myocardial abnormality, a brain abnormality, or a disorder related with oxygen activity abnormality, dysfunction, deficiency, and/or disruption.

In another aspect, the present disclosure encompasses use of the aforementioned HAPH analogs, metal chelates, and/or the compositions thereof in activating oxygen activity, in inducing DNA scission, and/or in inducing oxygen radical formulation in a subject in need thereof. The subject is having or suspected of having a cancer, a tumor, a blood pool, a thrombus, an inflammation, a hypoxia, a myocardial abnormality, a brain abnormality, or a disorder related with oxygen activity abnormality, dysfunction, deficiency, and/or disruption.

In another aspect, the present disclosure encompasses a method of manufacture a medicament for modulating oxygen activity, for inducing DNA scission, and/or for inducing oxygen radical formulation. Such medicament comprises aforementioned HAPH analogs, metal chelates, with or without a carrier.

In another aspect, the present disclosure encompasses a tumor-imaging method and/or a theranostic method by administering the aforementioned metal chelate to a subject in need thereof. In one aspect, the metal chelate is a fluorescent metal chelate or a radioisotope metal chelate. In another aspect, the radioisotope metal chelate is used in PET, MRI, and/or CT. In another aspect, the radioisotope comprises of ⁴³Sc, ⁴⁴Sc, ⁴⁶Sc, ⁴⁷Sc, ⁴⁸Sc, ⁵⁵Co, ⁶⁰Cu, ⁶¹Cu, ⁶²Cu, ⁶⁷Cu, ⁶⁴Cu, ¹⁸F, ⁶⁶Ga, ⁶⁷Ga, ⁶⁸Ga, ¹⁸⁸Re, ¹¹¹In, ¹¹³In, ⁹⁰Y, ⁸⁶Y and ^{99m}Tc. In one aspect, the radioisotope is ⁶⁷Cu (II) or ⁶⁴Cu (II). In another aspect, the subject in need of such imaging has or is suspected of having a cancer, a tumor, a blood pool, a thrombus, an inflammation, a hypoxia, a myocardial abnormality, a brain abnormality, or a disorder related with oxygen activity abnormality, deficiency, dysfunction and/or disruption.

In another aspect, the present disclosure encompasses a kit comprises at least a container, and an instruction. In one aspect, the at least one container holding the aforementioned compounds, metal chelates, and/or compositions, and the instruction provide directions on practicing the method or use thereof.

In another aspect, the present disclosure encompasses a scalable synthesis of an intermediate

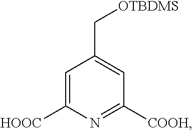 [SMILES: CC(C)(C)[Si](C)(C)OCc1cc(C(=O)O)nc(C(=O)O)c1]

comprising the steps showing in Scheme 8.

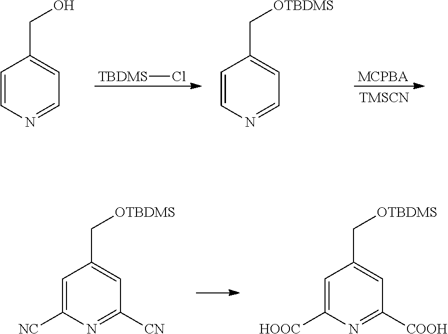

In another aspect, the scaled-up synthesis is used to synthesize one or more of the following intermediates:

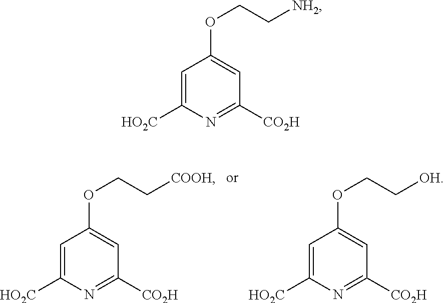 [SMILES: NCCOc1cc(C(=O)O)nc(C(=O)O)c1 ; O=C(O)CCOc1cc(C(=O)O)nc(C(=O)O)c1 ; O=C(O)c1cc(OCCO)cc(C(=O)O)n1]

### Reference to Color Figures

The application file contains at least one photograph executed in color. Copies of this patent application publication with color photographs will be provided by the Office upon request and payment of the necessary fee.

### BRIEF DESCRIPTION OF THE FIGURES

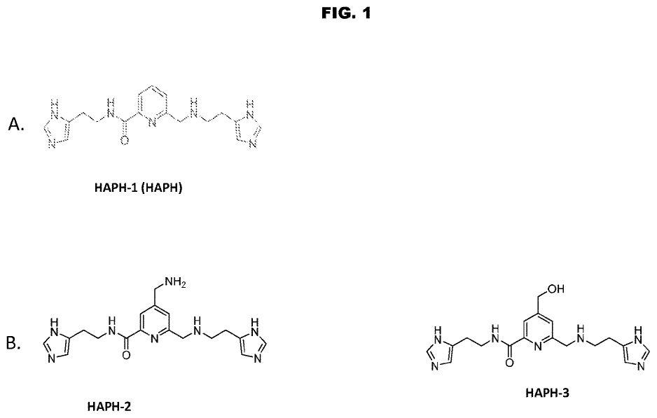

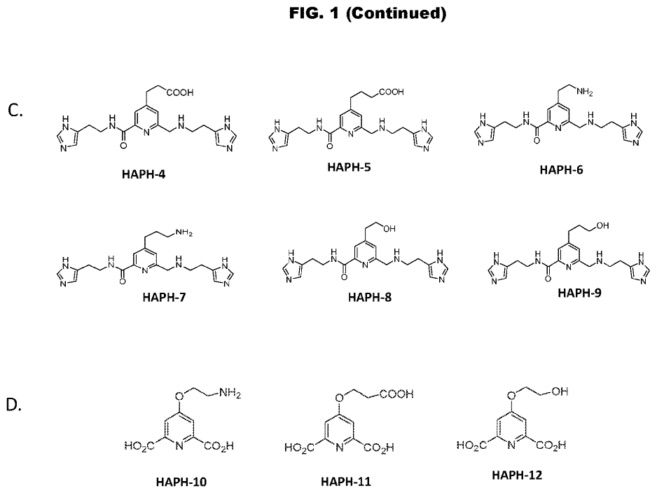

FIGS. 1A-D are structures of some exemplary HAPH analogs according to the present disclosure.

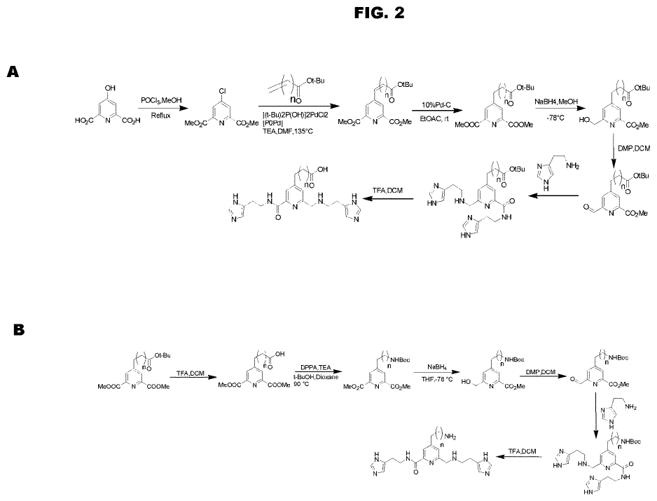

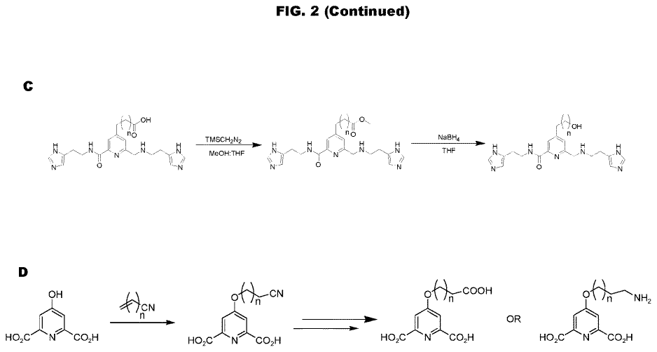

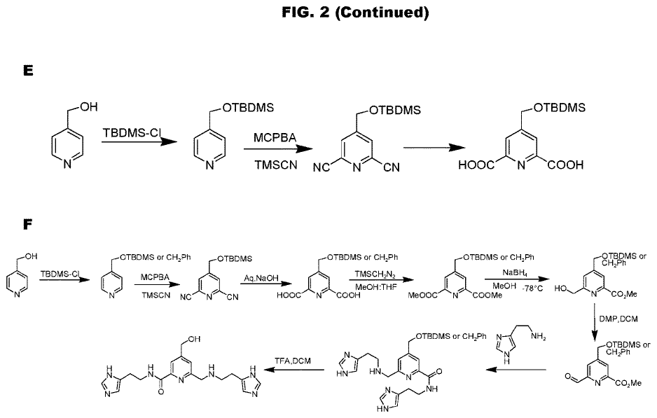

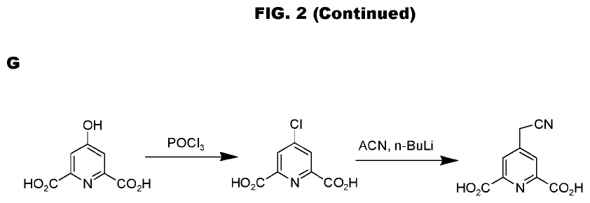

FIGS. 2A-G are schematic illustrations of exemplary processes for synthesizing HAPH analogs according to the present disclosure.

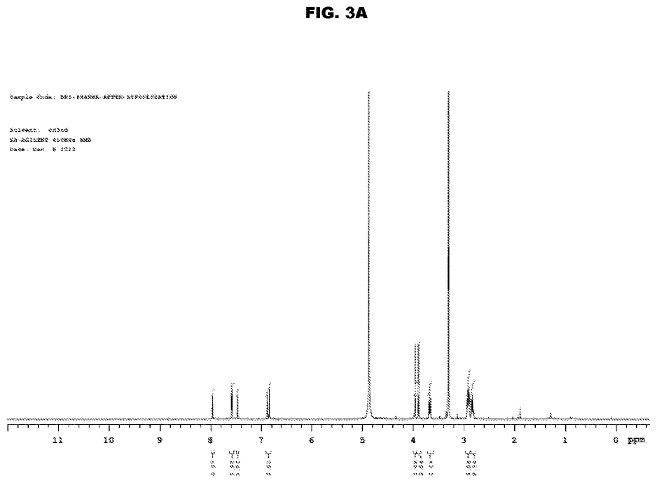

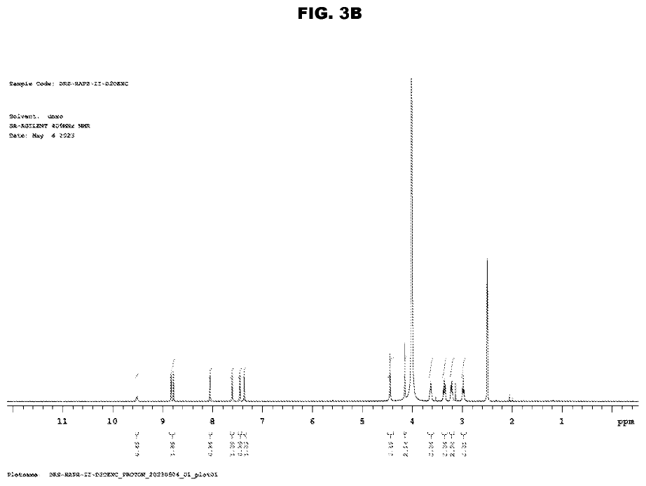

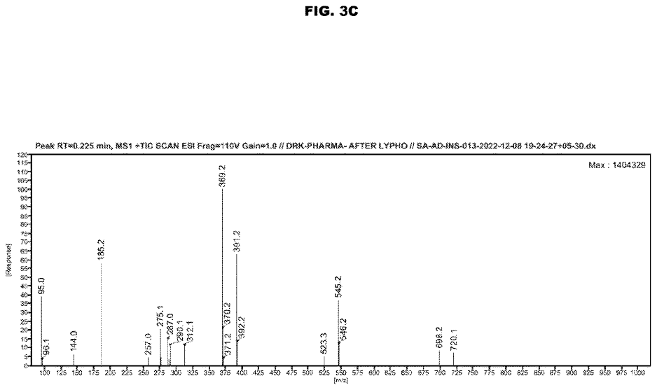

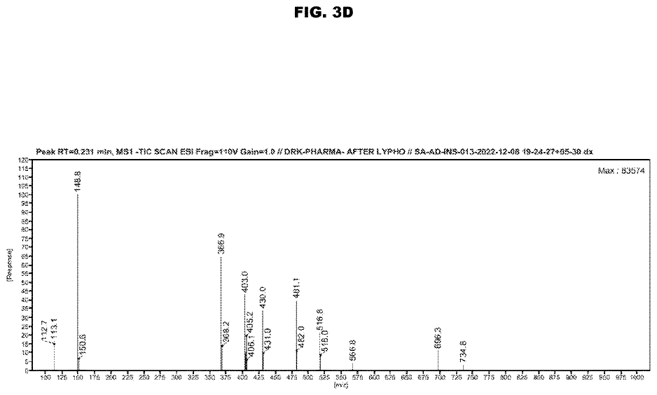

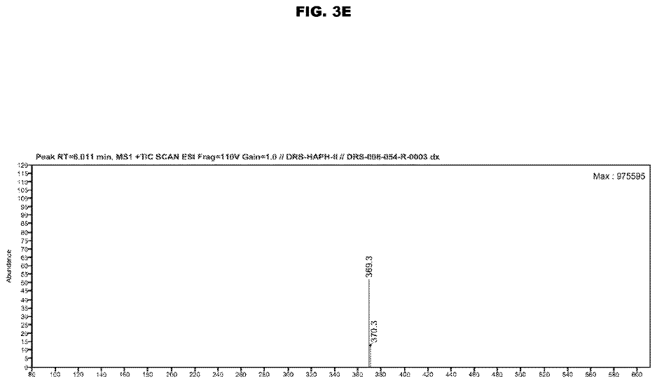

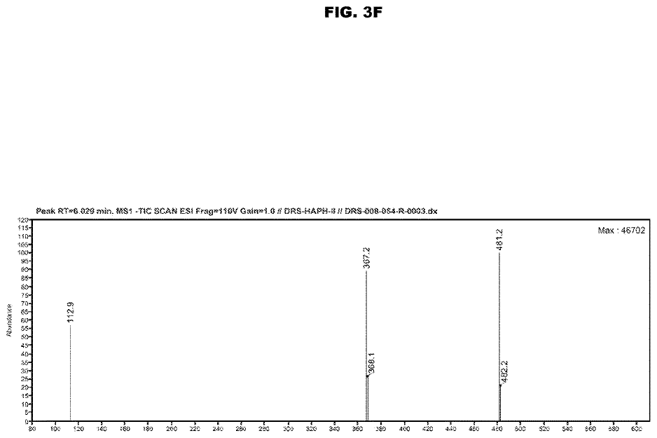

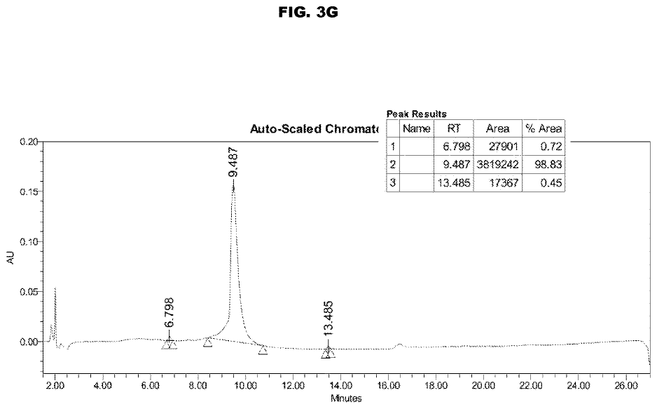

FIGS. 3A-3G are characterizations of HAPH-2. FIG. 3A is the ¹H NMR of HAPH-2; FIG. 3B is the ¹H NMR of HAPH-2 with D₂O exchange; FIG. 3C is the Mass Spectroscopy (MS) of HAPH-2 at RT=0.225 min; FIG. 3D is the MS of HAPH-2 at RT=0.231 min; FIG. 3E is the Mass Spectroscopy (MS) of HAPH-2 at RT=6.011 min; FIG. 3F is the LCMS of HAPH-2 at RT=6.029 min; FIG. 3G is the HPLC of HAPH-2 at RT=0.231 min, under the conditions of buffers A being 0.1% ammonia solution in water and B being acetonitrile; column being x-bridge shield rp 150*4.6*3.5 μm; gradient progression t/b being 0/10, 2/10, 15/50, 25/95, 25.5/10, 28/10.

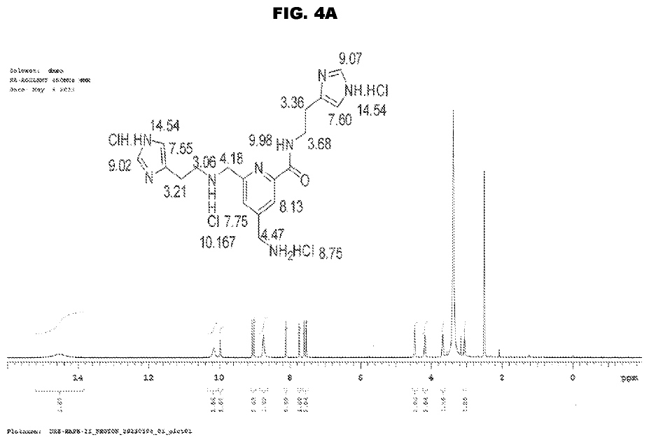

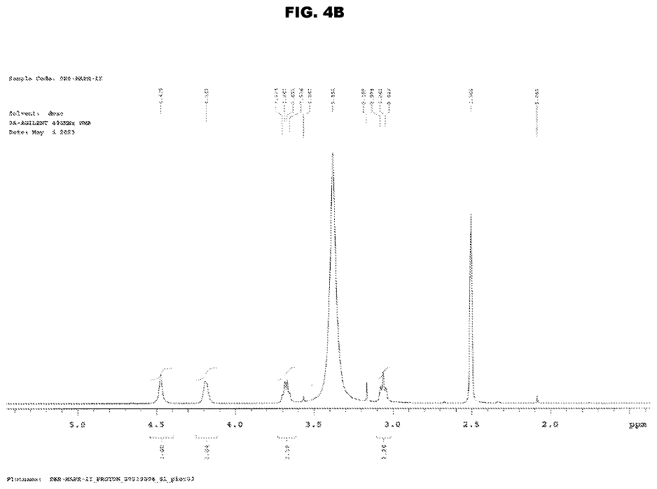

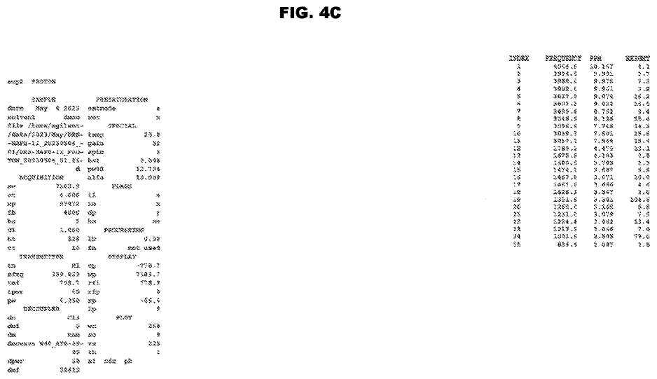

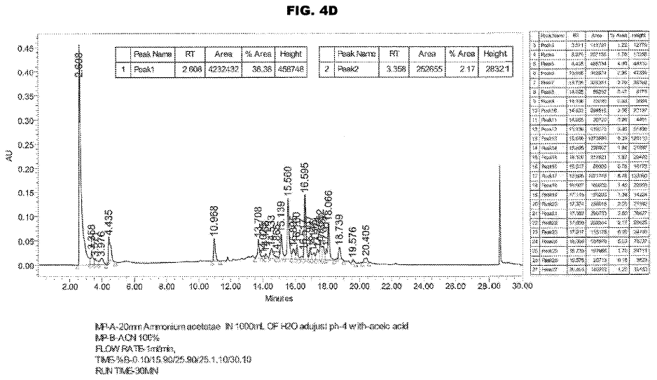

FIGS. 4A-4D are the characterization of HAPH-2 HCl. FIG. 4A is the 1H NMR confirmation of HAPH-2 HCl synthesis; FIG. 4B is the ¹H NMR of HAPH-2; FIG. 4C is the ¹H NMR reads of HAPH-2 HCl; FIG. 4D is the HPLC of HAPH-2 HCl with peak table.

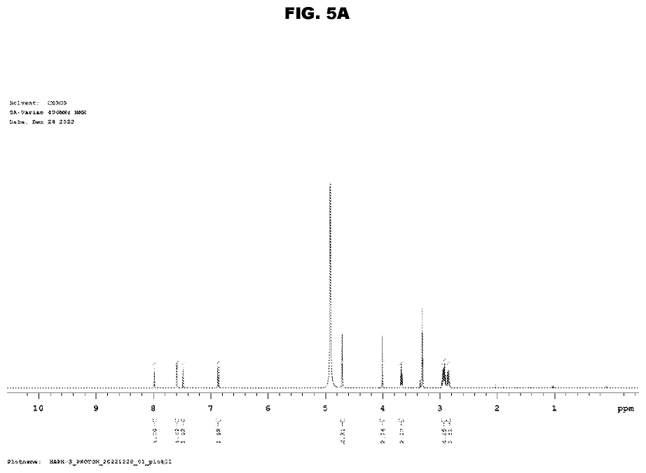

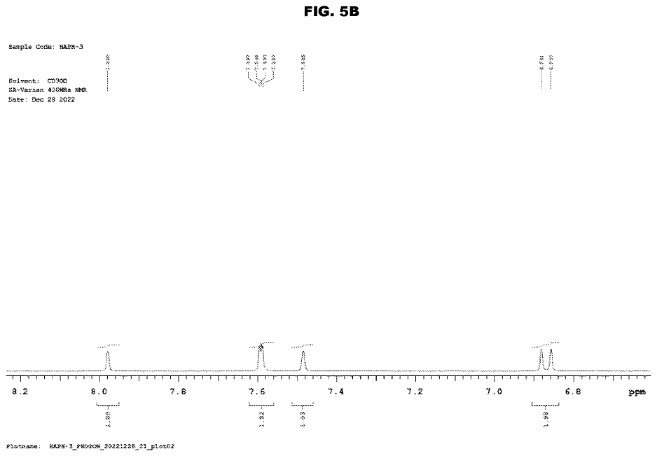

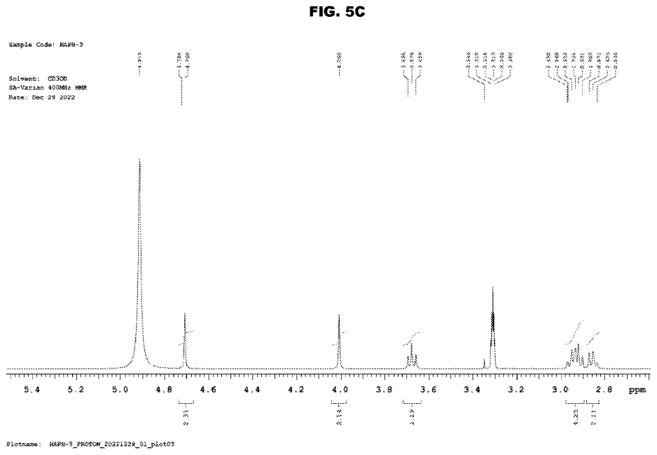

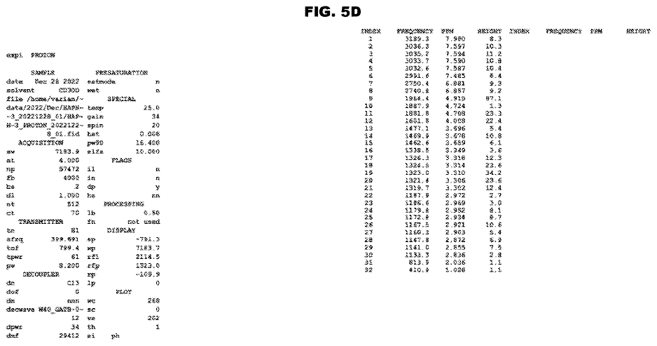

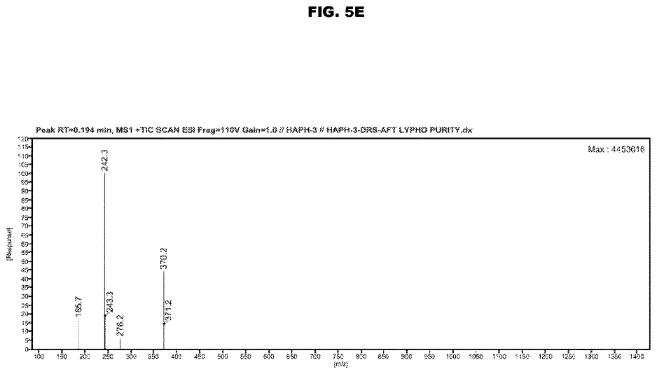

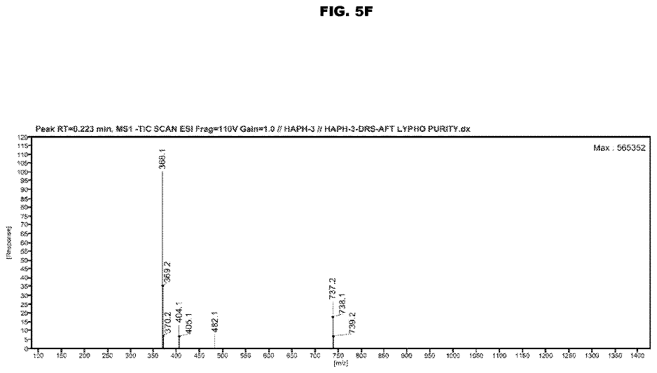

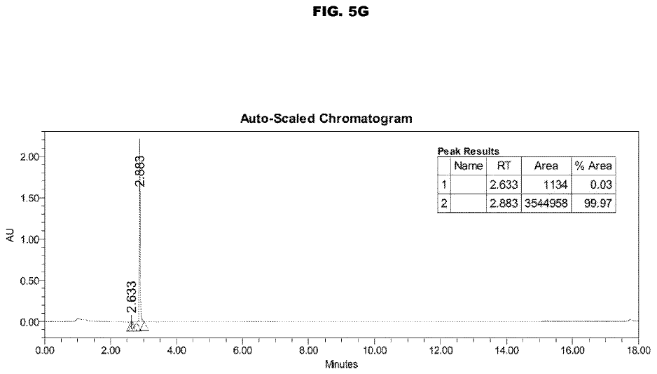

FIGS. 5A-5G are the characterization of HAPH-3. FIG. 5A is the 1H NMR of HAPH-3 (Plot 1); FIG. 5B is the ¹H NMR of HAPH-3 (Plot 2); FIG. 5C is the ¹H NMR of HAPH-3 (Plot 3); FIG. 5D is the ¹H NMR reads of HAPH-3; FIG. 5E is the Mass Spectroscopy (MS) of HAPH-3 at RT=0.194 min; FIG. 5F is the Mass Spectroscopy (MS) of HAPH-3 at RT=0.223 min; FIG. 5G is the HPLC of HAPH-3 performed under the conditions of Mobile Phase A being 0.1% TFA in water, phase B being ACN; Column being XBRIDGE SHIELD RP (150*4.6*3.5μ); Grad Prog being T/B: 0/10, 1/10, 12/95, 15/95, 15.5/10, 18/10; Flow being ml/min-1.0; and Runtime at 18.

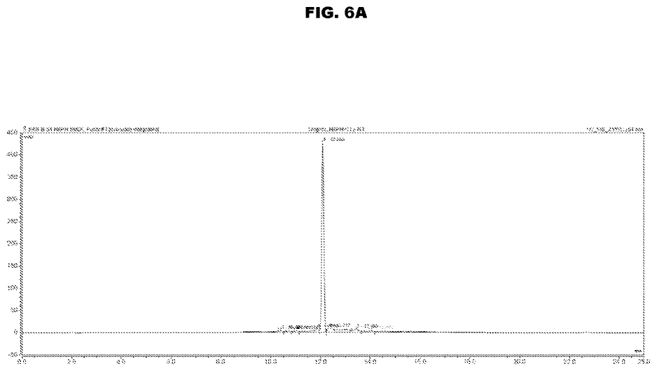

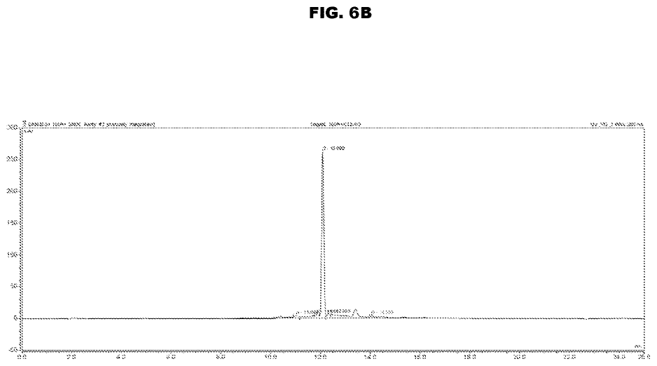

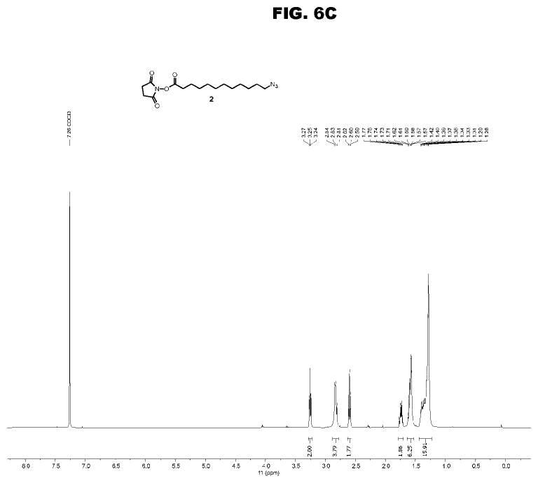

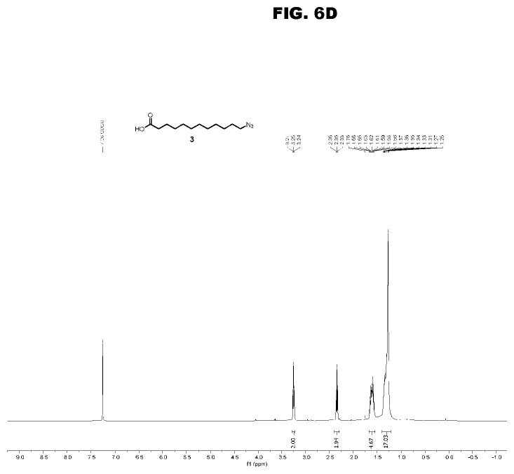

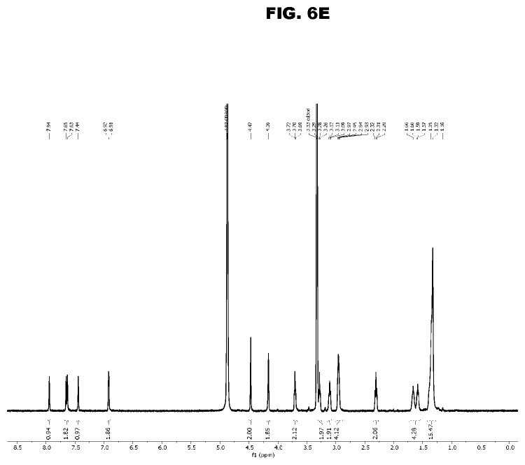

FIGS. 6A-6E are the characterization of HAPH-2 Azide. FIG. 6A provides HPLC chromatogram of HAPH-2 Azide monitored at 254 nm with elution time being 12.083 min. FIG. 6B provides HPLC chromatogram of HAPH-2 Azide monitored at 280 nm with elution time being 12.083 min. FIG. 6C is the ¹H NMR Spectrum of Intermediate 2 of HAPH-2 Azide. FIG. 6D is the ¹H NMR Spectrum of Intermediate 3 of HAPH-2 Azide. FIG. 6E is the ¹H NMR Spectrum of HAPH-2 Azide.

### DETAILED DESCRIPTION

The present disclosure is based in part on the design and synthesis of novel HAPH analogs, their metal chelates. Another aspect of the present disclosure is based on the discovery that aforementioned HAPH analogs and their metal chelates have beneficial biological activities and may be useful in treating cancer, tumor, a blood pool, a thrombus, an inflammation, a hypoxia, a myocardial abnormality, a brain abnormality, or other disorders involving oxygen activities.

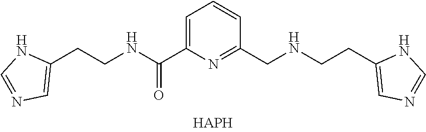 [SMILES: O=C(NCCc1cnc[nH]1)c1cccc(CNCCc2cnc[nH]2)n1]

The present disclosure relates to novel HAPH analogs, their metal chelates, for diagnostic, theranostic and/or therapeutic uses. The uses may include, but not limited to, treatment and/or prevention of cancer, tumor and/or other diseases. Such diseases may relate to oxygen activity abnormality, dysfunction, defects and/or deficiency, and may include, but not limited to cancer or tumor. The core molecule, HAPH, was first synthesized in 1988. HAPH, also coded as “HAPH-1” interchangeably in the present disclosure, was designed as an analogue of bleomycin (BLM), which is a glycopeptide antibiotic isolated from Streptomyces verticillus. BLM is a chemotherapeutic agent that exhibits antitumor activity toward at least Hodgkin's lymphoma and tumors of the head, neck, testis, and ovaries. Its antitumor action is produced by DNA strand scissions in the presence of Fe (II), Co (II), or Cu (II) and O₂. The metal ion binding region of BLM is similar to the Cu (II) binding site of human serum albumin. It has been previously demonstrated that the Fe (II) complex of HAPH has the ability to bind diatomic oxygen which then results in the generation of reactive oxygen species (e.g. Hydroxyl radical OH-and superoxide ion, O₂⁻). It has also been demonstrated that the Fe^{II}-HAPH-1 molecule binds relatively non-selectively to sequences in the minor groove of DNA. The generation of active oxygen species in intimate association to the DNA results in significant DNA strand cleavage. The ability of the Fe^{II}-HAPH-1 to cleave DNA may be useful in the design and application of antitumor and/or antitumor agents.

### I. HAPH Analogs

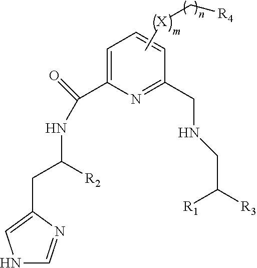

Various HAPH analogs are designed and synthesized. They contain the bleomycin core donors of imidazole, amide nitrogen, pyridine, and secondary amine joined to a terminal amine functional group. One aspect of the present disclosure encompasses a compound of Formula (I). In the structure, R₁ and R₃ each is independently selected from the substituted or unsubstituted group consisting of hydrogen, halogen, —NH₂, —C(O)NH₂, —C(O)OH, —C(O)Ome, —C(O)R₅, —C(O)NR₅, —OC(O)R₅, —C(O)OR₅, —(C₁-C₆ alkylene), —C₁-C₆ alkyl, —C₂-C₆ alkenyl, —C₂-C₆ alkynyl, —C₁-C₆ alkoxy, —C₁-C₆ heteroalkyl, —C₃-C₁₂ cycloalkyl, —C₆-C₁₄ aryl, —C₆-C₁₄ aryloxy, —C₆-C₁₄ aryl, 3-12 membered heterocyclyl, 3-12 membered heterocycloalkyl, 5-14 membered heteroaryl, —SH, —SMe, —SR₅, —S(O)₂R₅, —SR₅-aryl, and SR₅-aryl-O—R₅; and R₂ is selected from the substituted or unsubstituted group consisting of hydrogen, halogen, —C₁-C₆ alkyl, —C₂-C₆ alkenyl, and —C₂-C₆ alkynyl. In the Formula (I), X is —O—, —CH₂NR₅—, CH₂O—, —CH₂C(O)—, CH₂—NR₅—C(O)—, —NR₅—, —CH₂S—, or —S—; m is the number 0 or 1. And when m is 0, the methylene group is directly connected to the pyridine ring; n is zero or an integer of 1, 2, 3, 4, 5, 6, 8, 9, or 10 and R₄ is selected from the substituted or unsubstituted group consisting of hydrogen, hydroxyl, —NH₂, —C(O)NH₂, —NHC(O), —C(O)OH, —C(O)OMe, —C(O)R₅, —C(O)NR₅, —OC(O)R₅, —C(O)OR₅—, —N₃, —(CH₂)₁₀—N₃; —NH—C(O)—(CH₂)₁₁—N₃, —C₁-C₁₅ alkylazide, —C≡CH, —(CH₂)₁₀—C≡CH; —NH—C(O)—(CH₂)₁₁—C≡CH; —C₁-C₁₅—C≡CH; —SH, —SMe, —SR₅, —S(O)₂R₅, —SR₅-aryl, SR₅-aryl-O—R₅, —C₁-C₁₅ alkyl, —C₂-C₁₅ alkenyl, —C₂-C₁₅ alkynyl, and phosphate. Each R₅ is selected independently from the group consisting of C₁-C₆ alkyl, C₂-C₆ alkenyl, and C₂-C₆ alkynyl. Optionally, the compound of Formula (I) has the proviso that when m=n=0, R₁ is not selected from the group consisting of —NH₂, —SH, —SCH₃, SCH₂C₆H₄OCH₃, and 5-imidazolyl.

Another example of such HAPH analogs has the structure of Formula (II).

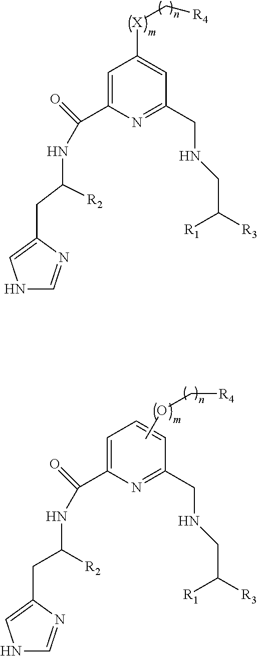

In some aspect, the compound can be expressed as Formula (III). In Formula (III), X is —O—, and m is the number 0 or 1. When m is 0, the methylene group is directly connected to the pyridine ring. The symbol “n” is zero or an integer of 1, 2, 3, 4, 5, 6, 7, 8, 9, or 10, and R₄ is selected from the substituted or unsubstituted group consisting of hydrogen, hydroxyl, —NH₂, —C(O)NH₂, —NHC(O), —C(O)OH, —C(O)OMe, —C(O)R₅, —C(O)NR₅, —OC(O)R₅, —C(O)OR₅—, —N₃, —(CH₂)₁₀—N₃; —NH—C(O)—(CH₂)₁₁—N₃, —C₁-C₁₅ alkylazide, —C≡CH, —(CH₂)₁₀—C≡CH; —NH—C(O)—(CH₂)₁₁—C≡CH; —C₁-C₁₅—C≡CH; —SH, —SMe, —SR₅, —S(O)₂R₅, —SR₅-aryl, SR₅-aryl-O—R₅, —C₁-C₁₅ alkyl, —C₂-C₁₅ alkenyl, —C₂-C₁₅ alkynyl, and phosphate. Each R₅ is selected independently from the group consisting of C₁-C₆ alkyl, C₂-C₆ alkenyl, and C₂-C₆ alkynyl.

In some aspect, Formula (III) has the proviso that m and no could not be both zero, in other words, Formula (III) may exclude those compounds that m=n=0. In yet another aspect, the compound of the present disclosure is of Formula (IV)

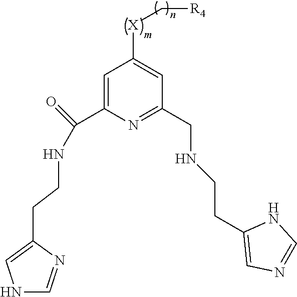

Another aspect of the present disclosure encompasses a compound corresponding to Formula (II) with the following substituents:

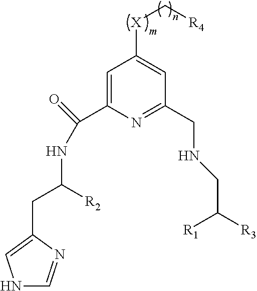

| NAME | R₁ | R₂ | R₃ | R₄ | X | m | n |
|---|---|---|---|---|---|---|---|
| HAPH-2 | 5-imidazolyl | H | H | NH₂ | N/A | 0 | 1 |
| HAPH-2 | 5-imidazolyl | H | H | NH₂ HCl | N/A | 0 | 1 |
| HCl Salt | | | | | | | |
| HAPH-2 | 5-imidazolyl | H | H | N₃ | CH₂—NHC(O) | 0 | 11 |
| Azide | | | | | | | |
| HAPH-2 | 5-imidazolyl | H | H | CΞC | CH₂—NHC(O) | 0 | 11 |
| Ethyne | | | | | | | |
| HAPH-2 | 5-imidazolyl | H | H | N₃ | N/A | 0 | 1 |
| Methylene | | | | | | | |
| Azide | | | | | | | |
| HAPH-3 | 5-imidazolyl | H | H | OH | N/A | 0 | 1 |
| HAPH-4 | 5-imidazolyl | H | H | COOH | N/A | 0 | 2 |
| HAPH-5 | 5-imidazolyl | H | H | COOH | N/A | 0 | 3 |
| HAPH-6 | 5-imidazolyl | H | H | NH₂ | N/A | 0 | 2 |
| HAPH-7 | 5-imidazolyl | H | H | NH₂ | N/A | 0 | 3 |
| HAPH-8 | 5-imidazolyl | H | H | OH | N/A | 0 | 2 |
| HAPH-9 | 5-imidazolyl | H | H | OH | N/A | 0 | 3 |
| HAPH-10 | 5-imidazolyl | H | H | NH₂ | O | 1 | 2 |
| HAPH-11 | 5-imidazolyl | H | H | COOH | O | 1 | 2 |
| HAPH-12 | 5-imidazolyl | H | H | OH | O | 1 | 2 |
| HAPH-13 | 5-imidazolyl | H | H | COOH | N/A | 0 | 1 |
| AMPHIS-1N | —NH₂ | —C(O)OCH₃ | H | NH₂ | N/A | 0 | 1 |
| AMPHIS-1O | —NH₂ | —C(O)OCH₃ | H | OH | N/A | 0 | 1 |
| AMPHIS-1A | —NH₂ | —C(O)OCH₃ | H | COOH | N/A | 0 | 1 |
| PYML-1N | NH₂ | —C(O)OH | —C(O)NH₂ | NH₂ | N/A | 0 | 1 |
| PYML-1O | NH₂ | —C(O)OH | —C(O)NH₂ | OH | N/A | 0 | 1 |
| PYML-1A | NH₂ | —C(O)OH | —C(O)NH₂ | COOH | N/A | 0 | 1 |
| SAPH-1N | —SMe | H | H | NH₂ | N/A | 0 | 1 |
| SAPH-1O | —SMe | H | H | OH | N/A | 0 | 1 |
| SAPH-1A | —SMe | H | H | COOH | N/A | 0 | 1 |
| SAPH-2N | —SCH₂C₆H₄Ome | H | H | NH₂ | N/A | 0 | 1 |
| SAPH-2O | —SCH₂C₆H₄Ome | H | H | OH | N/A | 0 | 1 |
| SAPH-2A | —SCH₂C₆H₄Ome | H | H | COOH | N/A | 0 | 1 |
| SAPH-3N | —SH | H | H | NH₂ | N/A | 0 | 1 |
| SAPH-3O | —SH | H | H | OH | N/A | 0 | 1 |
| SAPH-3A | —SH | H | H | COOH | N/A | 0 | 1 |

Each and every functional group may optionally be substituted or unsubstituted. Suitable substituents include, but are not limited to, C1-C6 alkyl, halogen, —CN, —NO2, —N3, C2-6 alkenyl, C2-6 alkynyl, —OR, —NH2, or —SR, R being hydrogen, halogen, —CN, —NO2, —N3, acyl, C1-6 alkyl, C2-6 alkenyl, C2-6 alkynyl.

Some exemplary compounds of the present disclosure are given in FIG. 1A-D. Some exemplary synthesis schemes of the present disclosure are given in FIG. 2A-G.

### II. Metal Chelates of HAPH Analogs

Another aspect of the present disclosure provides metal chelates of HAPH analogs. The term metal chelate encompasses any coordination or complex compounds consisting of a central metal atom attached to an organic molecule, optionally forming a cyclic or ring structure. In one aspect, the metal is a transition metal. In another aspect, the metal comprises, but not limited to, Fe, Cu, Cr, Mn, Co, Sc, Ni, Zn, Ag, Re, Y, or Tc. In yet another aspect, the metal may be in a form of a metal ion, comprising, but not limited to, Fe (II), Fe (III), Cu (II), Cr (III), Cr (VI), Mn (II), Mn (III), Mn (VI), Co (II), Co (III), Sc (II), Ni (II), Zn (II), Ga (III), In (IIII), Y (III), or Ag (I). Further, the metal may be an isotope form. The isotope may be a stable or an unstable isotope. The isotope may be a radioactive or non-radioactive isotope.

The present disclosure comprises a metal chelate of a compound of Formula (V), wherein the metal M is a transition metal, for example, M may be selected from the group comprising of Fe, Cu, Cr, Mg, Mn, Co, Sc, Ni, Zn, Ag, Re, Ga, In, Y, or Tc;

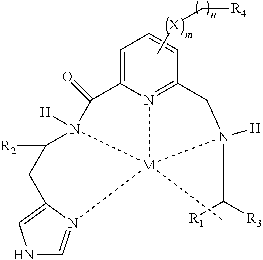

In Formula (V), the dashed bond to metal M is a chelate bond, indicating a type of bonding of ions and molecules to metal ions. It involves the formation or presence of two or more separate coordinate bonds between a polydentate (multiple bonded) ligand and a single central metal atom. When such bond is directed to a space between two substituents, such as R₁ and R₃ illustrated in Formula (V), the bond is to be interpreted as any viable bond formed by M with a viable atom of R₁ and R₃. R₁ and R₃ each is independently selected from the substituted or unsubstituted group consisting of hydrogen, halogen, —NH₂, —C(O)NH₂, —C(O)OH, —C(O)Ome, —C(O)R₅, —C(O)NR₅, —OC(O)R₅, —C(O)OR₅, —(C₁-C₆ alkylene), —C₁-C₆ alkyl, —C₂-C₆ alkenyl, —C₂-C₆ alkynyl, —C₁-C₆ alkoxy, —C₁-C₆ heteroalkyl, —C₃-C₁₂ cycloalkyl, —C₆-C₁₄ aryl, —C₆-C₁₄ aryloxy, —C₁₄ aryl, 3-12 membered heterocyclyl, 3-12 membered heterocycloalkyl, 5-14 membered heteroaryl, —SH, —SMe, —SR₅, —S(O)₂R₅, —SR₅-aryl, and SR₅-aryl-O—R₅. R₂ is selected from the substituted or unsubstituted group consisting of hydrogen, halogen, —C₁-C₆ alkyl, —C₂-C₆ alkenyl, and —C₂-C₆ alkynyl. In Formula (V), the motif Xm-(CH₂)n-R₄ can be either at para- or meta-position relative to Nitrogen on the pyridine ring. X is —O—, —CH₂NR₅—, CH₂O—, —CH₂C(O)—, CH₂—NR₅—C(O)—, —NR₅—, —CH₂S—, or —S—; and m is the number 0 or 1; and when m is 0, the methylene group is directly attached to the pyridine ring. Further, n is zero or an integer of 1, 2, 3, 4, 5, 6, 7, 8, 9, or 10; and R₄ is selected from the substituted or unsubstituted group consisting of hydrogen, hydroxyl, —NH₂, —C(O)NH₂, —NHC(O), —C(O)OH, —C(O)OMe, —C(O)R₅, —C(O)NR₅, —OC(O)R₅, —C(O)OR₅—, —N₃, —(CH₂)₁₀—N₃; —NH—C(O)—(CH₂)₁₁—N₃, —C₁-C₁₅ alkylazide, —C≡CH, —(CH₂)₁₀—C≡CH; —NH—C(O)—(CH₂)₁₁—C≡CH; —C₁-C₁₅—C≡CH; —SH, —SMe, —SR₅, —S(O)₂R₅, —SR₅-aryl, SR₅-aryl-O—R₅, —C₁-C₁₅ alkyl, —C₂-C₁₅ alkenyl, —C₂-C₁₅ alkynyl, and phosphate. Each R₅ is independently selected from the group consisting of C₁-C₆ alkyl, C₂-C₆ alkenyl, and C₂-C₆ alkynyl.

The present disclosure may optionally exclude the metal chelates of Formula (V), when metal is Fe or Cu, m=n=0, and R₁ is —SH, SMe, —SCH₂C₆H₄Ome or 5-imidazolyl. In other words, the present disclosure has the proviso clause that when metal is Fe or Cu, m=n=0, the metal chelates would not include those R₁ is —SH, SMe, —SCH₂C₆H₄OMe or 5-imidazolyl.

The present disclosure encompasses the metal chelate of Formula (V), and the metal is a transition metal. In one aspect, the metal is an ion form and the ion comprises Fe (II), Fe (III), Cu (II), Cr (III), Cr (VI), Mg (II), Mn (II), Mn (III), Mn (VI), Co (II), Co (III), Sc (II), Ni (II), Zn (II), Ga (III), In (IIII), Y (III), and Ag (I). In another aspect, the metal is an isotope form or a radioisotope form. In another aspect, the metal chelate is of Formula (VI).

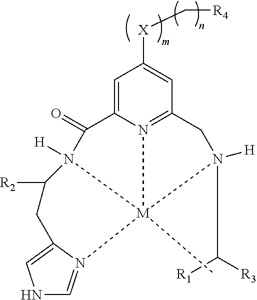

| NAME | R₁ | R₂ | R₃ | R₄ | X | m | n |
|---|---|---|---|---|---|---|---|
| AMPHIS | —NH₂ | —C(O)OCH₃ | H | H | N/A | 0 | 0 |
| PYML | NH₂ | —C(O)OH | —C(O)NH₂ | H | N/A | 0 | 0 |
| SAPH-1 | —SMe | H | H | H | N/A | 0 | 0 |
| SAPH-2 | —SCH₂C₆H₄Ome | H | H | H | N/A | 0 | 0 |
| SAPH-3 | —SH | H | H | H | N/A | 0 | 0 |
| HAPH-1 | 5-imidazolyl | H | H | H | N/A | 0 | 0 |
| HAPH-2 | 5-imidazolyl | H | H | NH₂ | N/A | 0 | 1 |
| HAPH-2 | 5-imidazolyl | H | H | NH₂ HCl | N/A | 0 | 1 |
| HCl Salt | | | | | | | |
| HAPH-2 | 5-imidazolyl | H | H | N₃ | CH₂—NHC(O) | 0 | 11 |
| Azide | | | | | | | |
| HAPH-2 | 5-imidazolyl | H | H | CΞC | CH₂—NHC(O) | 0 | 11 |
| Ethyne | | | | | | | |
| HAPH-2 | 5-imidazolyl | H | H | N₃ | N/A | 0 | 1 |
| Methylene | | | | | | | |
| Azide | | | | | | | |
| HAPH-3 | 5-imidazolyl | H | H | OH | N/A | 0 | 1 |
| HAPH-4 | 5-imidazolyl | H | H | COOH | N/A | 0 | 2 |
| HAPH-5 | 5-imidazolyl | H | H | COOH | N/A | 0 | 3 |
| HAPH-6 | 5-imidazolyl | H | H | NH₂ | N/A | 0 | 2 |
| HAPH-7 | 5-imidazolyl | H | H | NH₂ | N/A | 0 | 3 |
| HAPH-8 | 5-imidazolyl | H | H | OH | N/A | 0 | 2 |
| HAPH-9 | 5-imidazolyl | H | H | OH | N/A | 0 | 3 |
| HAPH-10 | 5-imidazolyl | H | H | NH₂ | O | 1 | 2 |
| HAPH-11 | 5-imidazolyl | H | H | COOH | O | 1 | 2 |
| HAPH-12 | 5-imidazolyl | H | H | OH | O | 1 | 2 |
| HAPH-13 | 5-imidazolyl | H | H | COOH | N/A | 0 | 1 |
| AMPHIS-1N | —NH₂ | —C(O)OCH₃ | H | NH₂ | N/A | 0 | 1 |
| AMPHIS-1O | —NH₂ | —C(O)OCH₃ | H | OH | N/A | 0 | 1 |
| AMPHIS-1A | —NH₂ | —C(O)OCH₃ | H | COOH | N/A | 0 | 1 |
| PYML-1N | NH₂ | —C(O)OH | —C(O)NH₂ | NH₂ | N/A | 0 | 1 |
| PYML-1O | NH₂ | —C(O)OH | —C(O)NH₂ | OH | N/A | 0 | 1 |
| PYML-1A | NH₂ | —C(O)OH | —C(O)NH₂ | COOH | N/A | 0 | 1 |
| SAPH-1N | —SMe | H | H | NH₂ | N/A | 0 | 1 |
| SAPH-1O | —SMe | H | H | OH | N/A | 0 | 1 |
| SAPH-1A | —SMe | H | H | COOH | N/A | 0 | 1 |
| SAPH-2N | —SCH₂C₆H₄Ome | H | H | NH₂ | N/A | 0 | 1 |
| SAPH-2O | —SCH₂C₆H₄Ome | H | H | OH | N/A | 0 | 1 |
| SAPH-2A | —SCH₂C₆H₄Ome | H | H | COOH | N/A | 0 | 1 |
| SAPH-3N | —SH | H | H | NH₂ | N/A | 0 | 1 |
| SAPH-3O | —SH | H | H | OH | N/A | 0 | 1 |
| SAPH-3A | —SH | H | H | COOH | N/A | 0 | 1 |

The metal chelates may elicit antitumor or anticancer action through DNA strand scissions and/or modulation of oxygen activity. The metal ion binding region of BLM is similar to the Cu (II) binding site of human serum albumin. It has been previously demonstrated that the Fe (II) complex of HAPH-1 has the ability to bind diatomic oxygen which results in the generation of reactive oxygen species (e.g. hydroxyl radical OH-and superoxide ion (O₂⁻). It has also been demonstrated that the Fe^{II}-HAPH-1 molecule bound relatively non-selectively to sequences in the minor groove of DNA. The generation of active oxygen species in intimate association to the DNA results in significant DNA strand cleavage. The ability of Fe^{II}-HAPH-1 chelate to cleave DNA is useful in the design and application of antitumor and/or anticancer agents.

Additionally, the metal chelates have utility in diagnostic imaging or testing. One limitation of ¹⁸F-FDG CT-PET imaging for cancer surveillance is the relatively short half-life (~109.7 minutes) of the ¹⁸F isotope. This makes production and delivery chronologically challenging especially in rural or remote areas. ⁶⁴Cu with a half-life of about 12.7 hours would be able to overcome this obstacle. ⁶⁷Cu is another option. Earlier studies on ⁶⁷Cu(II)-HAPH-1 and ⁶⁴Cu(II)-HAPH-1 showed favorable results towards medical imagining. Radioisotope ⁶⁷Cu is a 100% β-emitter that theoretically would have the ability to mediate DNA strand scission.

### III. Method of Making HAPH Analogs and Metal Chelates

The present disclosure encompasses methods of making HAPH analogs, and/or metal chelates thereof. These compounds may be synthesized through various schemes and involving custom-made intermediates as those provided in FIGS. 1A-1D and FIGS. 2A-2G. One example of such synthesis is shown in Scheme 1 below together with intermediates.

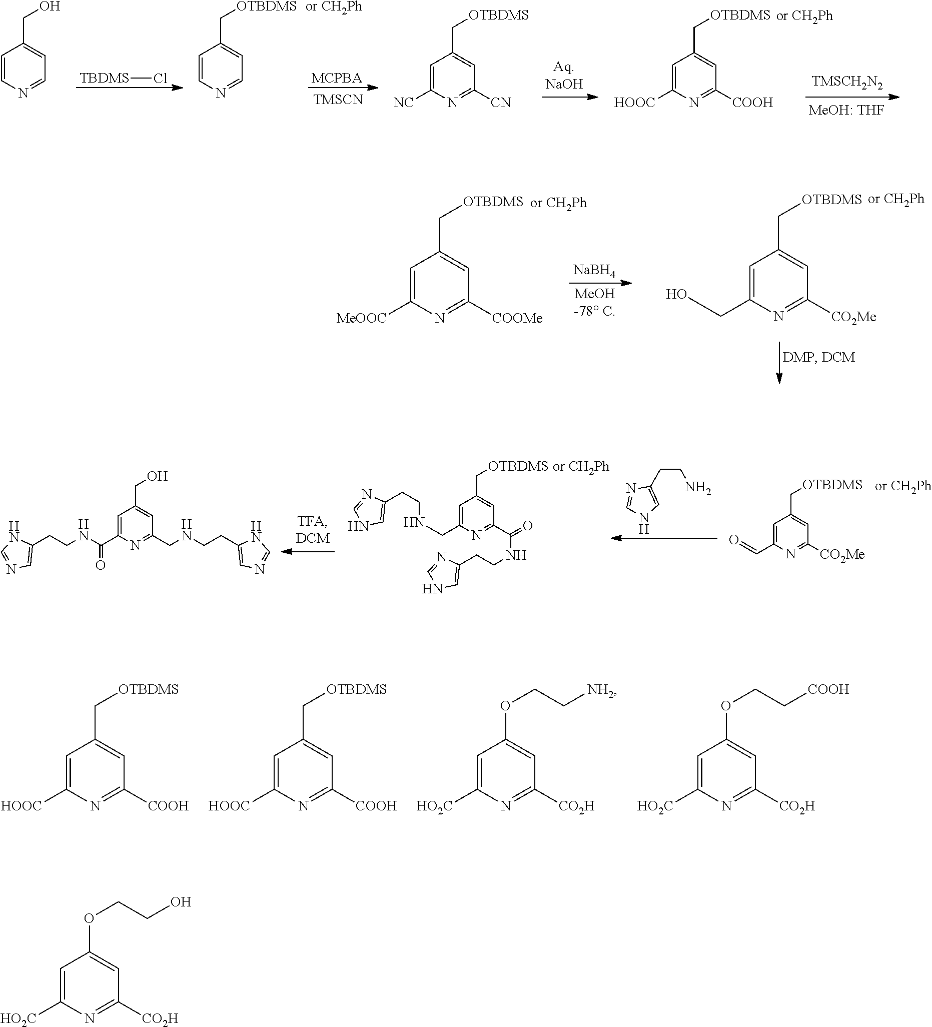

The HAPH analogs and metal chelates may be produced in lab amount and/or industrial quantities. Their availability for large-scale or industrial-scale synthesis is critical in drug discovery, clinical development, and commercialization for new small molecule medicines. A practical industrial-scale synthesis must be efficient, cost-effective, and reproducible. Further, all starting materials and reagents must be reliably available in bulk, or able to be produced on site in a safe and economical fashion. Additionally, all reagents, materials, conditions and equipment must meet the regulatory standards. These challenges make the large-scale synthesis a great hurdle to overcome and often require consideration even in the very beginning of drug discovery. The present disclosure is able to tackle this issue successfully and provides novel intermediates with industrial scale potential. Some of the intermediates are shown in Scheme 1, and their representative synthesis is given in Scheme 8.

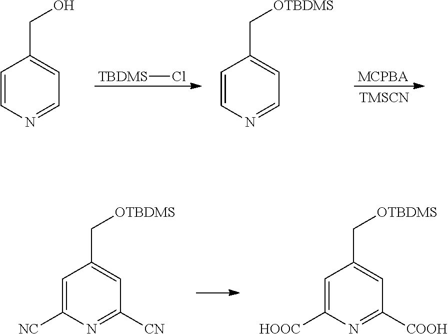

### IV. Methods of Use

The present disclosure encompasses uses and methods of use. The aforementioned compounds and metal chelates may be used in various medical fields, such as in disease therapy, in disease diagnosis, and/or as theranostics. One such use is to modulate oxygen activity in a subject, to induce DNA scission, and/or to induce oxygen radical formation in a subject. One method of use is to administer an amount sufficient to modulate oxygen activity, sufficient to induce DNA scission, and/or sufficient to induce oxygen radical formation in a subject by administering the compounds, the chelates, or their compositions thereof. The subject comprises, but not limited to, mammals and non-mammals. The subject may be a human being. The subject may be having or is suspect of having a cancer, a tumor, a blood pool, a thrombus, an inflammation, a hypoxia, a myocardial abnormality, a brain abnormality, or any other disorders related with oxygen activity.

Oxygen activity plays critical roles in many biological functions, including but not limited to DNA scission, and/or inducing the formation of oxygen radicals. Appropriate oxygen activity modulation may promote chemical reactions to trigger DNA scission in tumor cells, to degrade tumor polypeptides and polynucleotides by converting tumor proteins into peroxides that cleave the DNA backbone. The present disclosure encompasses HAPH analogs that are capable of inducing tumor DNA strand scissions, such as upon oxidation at the DNA backbones. Reactive oxygen species are widely generated in biological systems. Intracellular production of active oxygen species, such as—radical OH, O₂⁻ and H₂O₂, is associated with, among other things, the arrest of cell proliferation. Similarly, generation of oxidative stress in response to various external stimuli has been implicated in the activation of transcription factors and to the triggering of apoptosis. The present disclosure encompasses HAPH analogs and their metal chelates that are capable of initiating apoptosis signaling leading to tumor cell death, or to the activation of several proto-oncogenes and/or the activation of tumor suppressor genes.

The present disclosure provides methods of use by administering to a subject in need thereof an effective amount of the HAPH analogs, metal chelates, or the compositions thereof. Such use can be a therapeutic use, a diagnostic use, an imaging use, a theranostic use, or any combination thereof. Such use of the HAPH analogs, metal chelates, and/or the compositions may relate to activate oxygen activity, to induce DNA scission, and/or to induce oxygen radical formulation in a subject in need thereof. The effective amount of the HAPH analogs, metal chelates, or the compositions thereof, may be a pharmaceutical effective amount, a diagnostic effective amount, an imaging effective amount, a theranostic effective amount. The subject, such as a human being or other mammals, may be having or suspected of having a cancer, a tumor, a blood pool, a thrombus, an inflammation, a hypoxia, a myocardial abnormality, a brain abnormality, or a disorder related with oxygen activity abnormality, dysfunction, deficiency or disruption.

The present disclosure provides a method of manufacturing a medicament for activating oxygen activity, for inducing DNA scission, and/or for inducing oxygen radical formulation. Such medicament comprises aforementioned compounds, metal chelates, or compositions thereof.

The present disclosure provides a tumor-imaging method and/or a theranostic method by administering the metal chelates of HAPH analogs to a subject in need thereof. The metal chelate may be a fluorescent metal chelate or a radioisotope metal chelate. The radioisotope metal chelate may be used in Positron emission tomography (PET), Magnetic Resonance Imagine (MRI), and/or computed tomography (CT), and any other imaging or diagnostic instruments or tools, such as brain imaging, hypoxia imaging, inflammation imaging, thrombus imaging, blood pool and myocardial imaging. The radioisotope may comprise ⁴³Sc, ⁴⁴Sc, ⁴⁶Sc, ⁴⁷Sc, ⁴⁸Sc, ⁵⁵Co, ⁶⁰Cu, ⁶¹Cu, ⁶²Cu, ⁶⁷Cu, ⁶⁴Cu, ¹⁸F, ⁶⁶Ga, ⁶⁷Ga, ⁶⁸Ga, ¹⁸Re, ¹¹¹In, ¹¹³In, ⁹⁰Y, ⁸⁶Y and ^{99m}Tc. The radioisotope may comprise ⁶⁷Cu (II) or ⁶⁴Cu (II). The subject in need of such imaging has or is suspected of having a cancer, a tumor, a blood pool, a thrombus, an inflammation, a hypoxia, a myocardial abnormality, a brain abnormality, or a disorder related with oxygen activity abnormality, deficiency, dysfunction and/or disruption. The present disclosure also provides a tumor-imaging method by administering the metal chelate to a subject in need thereof. The administration may be parenteral, enteral, topical or transdermal. The administration may be through an injection. The injection includes, but not limited to, subcutaneous, intravenous, intramuscular, intrathecal, intrasternal, or infusion injections.

The present disclosure also provides a theranostics method by administering the HAPH analogs or their metal chelates to a subject. Theranostics is a treatment using diagnostic imaging to identify if target receptors are present on cancer cells, followed by precision radiation treatment that target these receptors. One advantage of the metal chelates in the present disclosure is that they are both biological active in terms of disease treatment, and also diagnostic probing due to the isotope labeling and tracing effect. Such theranostics use may be combined with imaging tools, such as PET, MRI, and/or CT.

### V. Diseases and Disorders Encompassed by the Present Disclosure

HAPH analogs, and/or metal chelates thereof may elicit various biological beneficial activities, including but not limited to, DNA scission, oxygen activity modulation, and beneficial radical formation. Therefore, the present disclosure encompasses the use of HAPH analogs and/or metal chelates thereof as cytotoxic compounds for treatment of any condition which requires arresting cell cycle and initiating cell apoptosis, and other degenerative diseases caused by oxidative radical stress or free radical toxicity in a subject. Such conditions may include cancer, neoplasm, tumor, a blood pool, a thrombus, an inflammation, a hypoxia, a myocardial abnormality, a brain abnormality, or any disorder related to oxygen activity abnormality, dysfunction, deficiency and/or disruption.

As it will be recognized by individuals skilled in the art, cancer as used throughout the instant disclosure may be one or more neoplasm or cancer, may be a solid tumor cancer and/or a soft tissue cancer. The solid tumor cancer comprises, but not limited to colorectal cancer, pancreatic cancer, primary liver cancers, kidney cancer, ovarian cancer, uterine cancer, lung cancer, breast cancer, prostate cancer, sarcomas, adipose tissue cancer, a subtype or any combination thereof. A soft tissue cancer or soft tissue sarcomas is understood as malignant neoplasms in the muscles, tendons, fat, blood vessels, lymphatic vessels, nerves and tissues around the joints. Normally, soft tissue sarcomas in adults can form in almost any part of the body, but they are more common in the head, neck, arms, legs, trunk and abdomen. Examples of soft tissue cancer are, but are not limited to, Solitary Fibrous Tumor (TFS), Hemangiopericytoma (HPC), Ewing's sarcoma, synovial sarcoma, rhabdomyosarcoma, and myxofibrosarcoma. The neoplasm may be malignant or benign, the cancer may be primary or metastatic; the neoplasm or cancer may be early stage or late stage. Non-limiting examples of neoplasms or cancers that may be treated include acute lymphoblastic leukemia, acute myeloid leukemia, adrenocortical carcinoma, AIDS-related cancers, AIDS-related lymphoma, anal cancer, appendix cancer, astrocytomas (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas), breast cancer, bronchial adenomas/carcinoids, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenström), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, sarcoma (Ewing family of tumors, Kaposi, soft tissue, uterine), Sézary syndrome, skin cancers (nonmelanoma, melanoma), skin carcinoma (Merkel cell), small cell lung cancer, small intestine cancer, soft tissue sarcoma, squamous cell carcinoma, squamous neck cancer with occult primary (metastatic), stomach cancer, supratentorial primitive neuroectodermal tumor (childhood), T-Cell lymphoma (cutaneous), testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), unknown primary site (adult, childhood), ureter and renal pelvis transitional cell cancer, urethral cancer, uterine cancer (endometrial), uterine sarcoma, vaginal cancer, visual pathway and hypothalamic glioma (childhood), vulvar cancer, Waldenström macroglobulinemia, and Wilms tumor (childhood). The cancer stages include advanced-stage, unresectable, or metastatic solid tumor cancers, including in subjects that have failed, or become intolerant, resistant, or refractory to an existing cancer therapy. The current methods may apply to cancer of various stages, and may apply to a cancer subject comorbidity with other diseases or disorders.

The method comprises administering a therapeutically effective, a theranostically effective, and/or diagnostic effective amount of HAPH analogs or their metal chelates to a subject when the subject is determined to have, is having, or is suspecting of having anyone of the above disclosed conditions or is determined to an eligible candidate for the therapy.

### VI. Compositions, Dosage Forms and Administration Regimen

One aspect of the disclosure encompasses a pharmaceutical composition or formulation for delivery of the HAPH analog and/or metal chelates thereof. A pharmaceutical composition or formulation comprises an effective amount of the active, and any pharmaceutically acceptable salt thereof.

Pharmaceutically acceptable salts of the compound of Formulas (I)-(IV) include, without limitation, acetate, aspartate, benzoate, bitartrate, citrate, formate, gluconate, glucuronate, glutamate, fumarate, hydrochloride, hydrobromide, hydroiodide, hypophosphite, isobutyrate, isocitrate, lactate, malate, maleate, meconate, methylbromide, methanesulfonate, monohydrate, mucate, nitrate, oxalate, phenylpropionate, phosphate, phthalate, propionate, pyruvate, salicylate, stearate, succinate, sulfate, tannate, tartrate, terephthalate, valerate, and the like.

The amount of compounds of Formula (I) to Formula (VI) in the composition, hereinafter collectively called “HAPH composition”, may be a therapeutically effective amount. As used herein “therapeutically effective amount” or “therapeutically effective dosage” refers to an amount that is effective to achieve a desired therapeutic result. In some embodiments, the desired therapeutic result is oxygen activity modulation and/or DNA scissions. For example, the therapeutically effective amount may be an amount that increases oxygen activity by at least 10%, preferably 20% or more, preferably 25% or more, preferably 30% or more, preferably 35% or more, preferably 40% or more, preferably 45% or more, preferably 50% or more, preferably 60% or more, preferably 70% or more, preferably 80% or more, preferably 90% or more. The therapeutically effective amount may be an amount that induces DNA scission by at least 10%, preferably 20% or more, preferably 25% or more, preferably 30% or more, preferably 35% or more, preferably 40% or more, preferably 45% or more, preferably 50% or more, preferably 60% or more, preferably 70% or more, preferably 80% or more, preferably 90% or more. The therapeutically effective amount may be an amount that induces oxygen radical formation by at least 10%, preferably 20% or more, preferably 25% or more, preferably 30% or more, preferably 35% or more, preferably 40% or more, preferably 45% or more, preferably 50% or more, preferably 60% or more, preferably 70% or more, preferably 80% or more, preferably 90% or more.

In some cases, the amount of compound of Formula (I) through Formula (VI) (hereinafter collectively called “HAPH composition”) present in the composition is more than about 1 μg. For example, the HAPH composition may comprise an amount of compounds of Formula (I) to (VI) of about 2 μg or more, about 5 μg or more, about 10 μg or more, about 100 μg or more, about 500 μg or more, about 1000 μg or more, about 1500 μg or more, about 2000 μg or more, about 2500 μg or more, about 3000 μg or more, about 3500 μg or more, about 4000 μg or more, about 4500 μg or more, about 5000 μg or more, about 5500 μg or more, about 6000 μg or more, about 6500 μg or more, about 7000 μg or more, about 7500 μg or more, about 8000 μg or more, about 8500 μg or more, about 9000 μg or more, about 9500 μg or more, about 10 mg or more, about 20 mg or more, about 30 mg or more, about 40 mg or more, about 50 mg or more, about 60 mg or more, about 70 mg or more, about 80 mg or more, about 90 mg or more, about 100 mg or more, about 150 mg or more, about 200 mg or more, about 250 mg or more, about 300 mg or more, about 350 mg or more, about 400 mg or more, about 450 mg or more, about 500 mg or more, about 550 mg or more, about 600 mg or more, about 650 mg or more, about 700 mg or more, about 800 mg or more, about 900 mg or more, or about 1 g or more.

Additionally or alternatively, the amount of compounds of Formula (I) to Formula (VI) in the HAPH composition may be about 0.01 wt. % to about 95 wt. %, about 0.1 wt. % to about 95 wt. %, about 1 wt. % to about 95 wt. %, about 5 wt. % to about 95 wt. %, about 10 wt. % to about 95 wt. %, about 15 wt. % to about 95 wt. %, about 20 wt. % to about 95 wt. %, about 30 wt. % to about 95 wt. %, about 40 wt. % to about 95 wt. %, about 50 wt. % to about 95 wt. %, about 60 wt. % to about 95 wt. %, about 70 wt. % to about 95 wt. %, about 80 wt. % to about 95 wt. %; about 0.01 wt. % to about 85 wt. %, about 0.1 wt. % to about 85 wt. %, about 1 wt. % to about 85 wt. %, about 5 wt. % to about 85 wt. %, about 10 wt. % to about 85 wt. %, about 15 wt. % to about 85 wt. %, about 20 wt. % to about 85 wt. %, about 30 wt. % to about 85 wt. %, about 40 wt. % to about 85 wt. %, about 50 wt. % to about 85 wt. %, about 60 wt. % to about 85 wt. %, about 70 wt. % to about 85 wt. %; about 0.01 wt. % to about 75 wt. % about 0.1 wt. % to about 75 wt. %, about 1 wt. % to about 75 wt. %, about 5 wt. % to about 75 wt. %, about 10 wt. % to about 75 wt. %, about 15 wt. % to about 75 wt. %, about 20 wt. % to about 75 wt. %, about 30 wt. % to about 75 wt. %, about 40 wt. % to about 75 wt. %, about 50 wt. % to about 75 wt. %, about 60 wt. % to about 75 wt. %; about 0.01 wt. % to about 65 wt. %, about 0.1 wt. % to about 65 wt. %, about 1 wt. % to about 65 wt. %, about 5 wt. % to about 65 wt. %, about 10 wt. % to about 65 wt. %, about 15 wt. % to about 65 wt. %, about 20 wt. % to about 65 wt. %, about 30 wt. % to about 65 wt. %, about 40 wt. % to about 65 wt. %, about 50 wt. % to about 65 wt. %; about 0.01 wt. % to about 55 wt. %, about 0.1 wt. % to about 55 wt. %, about 1 wt. % to about 55 wt. %, about 5 wt. % to about 55 wt. %, about 10 wt. % to about 55 wt. %, about 15 wt. % to about 55 wt. %, about 20 wt. % to about 55 wt. %, about 30 wt. % to about 55 wt. %, about 40 wt. % to about 55 wt. %; about 0.01 wt. % to about 45 wt. %, about 0.1 wt. % to about 45 wt. %, about 1 wt. % to about 45 wt. %, about 5 wt. % to about 45 wt. %, about 10 wt. % to about 45 wt. %, about 15 wt. % to about 45 wt. %, about 20 wt. % to about 45 wt. %, about 30 wt. % to about 45 wt. %; about 0.01 wt. % to about 35 wt. %, about 0.1 wt. % to about 35 wt. %, about 1 wt. % to about 35 wt. %, about 5 wt. % to about 35 wt. %, about 10 wt. % to about 35 wt. %, about 15 wt. % to about 35 wt. %, about 20 wt. % to about 35 wt. %; about 0.01 wt. % to about 25 wt. %, about 0.1 wt. % to about 25 wt. %, about 1 wt. % to about 25 wt. %, about 5 wt. % to about 25 wt. %, about 10 wt. % to about 25 wt. %; about 0.1 wt. % to about 15 wt. %, about 1 wt. % to about 15 wt. %, about 5 wt. % to about 15 wt. %, about 10 wt. % to about 15 wt. %; about 0.01 wt. % to about 25 wt. %, about 0.1 wt. % to about 10 wt. %, about 1 wt. % to about 10 wt. %, or about 5 wt. % to about 10 wt. %, including ranges and subranges thereof, based on the total weight of the HAPH composition.

Alternatively or additionally, the HAPH composition may include other anti-tumor or anti-cancer actives, or any combination thereof in an amount of about 0.01 wt. % to about 95 wt. %, about 0.1 wt. % to about 95 wt. %, about 1 wt. % to about 95 wt. %, about 5 wt. % to about 95 wt. %, about 10 wt. % to about 95 wt. %, about 15 wt. % to about 95 wt. %, about 20 wt. % to about 95 wt. %, about 30 wt. % to about 95 wt. %, about 40 wt. % to about 95 wt. %, about 50 wt. % to about 95 wt. %, about 60 wt. % to about 95 wt. %, about 70 wt. % to about 95 wt. %, about 80 wt. % to about 95 wt. %; about 0.01 wt. % to about 85 wt. %, about 0.1 wt. % to about 85 wt. %, about 1 wt. % to about 85 wt. %, about 5 wt. % to about 85 wt. %, about 10 wt. % to about 85 wt. %, about 15 wt. % to about 85 wt. %, about 20 wt. % to about 85 wt. %, about 30 wt. % to about 85 wt. %, about 40 wt. % to about 85 wt. %, about 50 wt. % to about 85 wt. %, about 60 wt. % to about 85 wt. %, about 70 wt. % to about 85 wt. %; about 0.01 wt. % to about 75 wt. %, about 0.1 wt. % to about 75 wt. %, about 1 wt. % to about 75 wt. %, about 5 wt. % to about 75 wt. %, about 10 wt. % to about 75 wt. %, about 15 wt. % to about 75 wt. %, about 20 wt. % to about 75 wt. %, about 30 wt. % to about 75 wt. %, about 40 wt. % to about 75 wt. %, about 50 wt. % to about 75 wt. %, about 60 wt. % to about 75 wt. %; about 0.01 wt. % to about 65 wt. % about 0.1 wt. % to about 65 wt. %, about 1 wt. % to about 65 wt. %, about 5 wt. % to about 65 wt. %, about 10 wt. % to about 65 wt. %, about 15 wt. % to about 65 wt. %, about 20 wt. % to about 65 wt. %, about 30 wt. % to about 65 wt. %, about 40 wt. % to about 65 wt. %, about 50 wt. % to about 65 wt. %; about 0.01 wt. % to about 55 wt. %, about 0.1 wt. % to about 55 wt. %, about 1 wt. % to about 55 wt. %, about 5 wt. % to about 55 wt. %, about 10 wt. % to about 55 wt. %, about 15 wt. % to about 55 wt. %, about 20 wt. % to about 55 wt. %, about 30 wt. % to about 55 wt. %, about 40 wt. % to about 55 wt. %; about 0.01 wt. % to about 45 wt. %, about 0.1 wt. % to about 45 wt. %, about 1 wt. % to about 45 wt. %, about 5 wt. % to about 45 wt. %, about 10 wt. % to about 45 wt. %, about 15 wt. % to about 45 wt. %, about 20 wt. % to about 45 wt. %, about 30 wt. % to about 45 wt. %; about 0.01 wt. % to about 35 wt. %, about 0.1 wt. % to about 35 wt. %, about 1 wt. % to about 35 wt. %, about 5 wt. % to about 35 wt. %, about 10 wt. % to about 35 wt. %, about 15 wt. % to about 35 wt. %, about 20 wt. % to about 35 wt. %; about 0.01 wt. % to about 25 wt. %, about 0.1 wt. % to about 25 wt. %, about 1 wt. % to about 25 wt. %, about 5 wt. % to about 25 wt. %, about 10 wt. % to about 25 wt. %; about 0.01 wt. % to about 15 wt. % about 0.1 wt. % to about 15 wt. %, about 1 wt. % to about 15 wt. %, about 5 wt. % to about 15 wt. %, about 10 wt. % to about 15 wt. %; about 0.01 wt. % to about 10 wt. % about 0.1 wt. % to about 10 wt. %, about 1 wt. % to about 10 wt. %, or about 5 wt. % to about 10 wt. %, including ranges and subranges thereof, based on the total weight of the HAPH composition.

Additionally or alternatively, the HAPH compositions may be formulated to have a weight ratio of at least one compound of Formula (I) to Formula (VI), in combination with another HAPH analog, or in combination with any metal chelates. The different actives in the formulation may have a weight ratio ranges from 1:100 to 100:1, 1:50 to 50:1, 1:20 to 20:1 1:10 to 10:1, 1:9 to 10:1, 1:8 to 10:1, 1:7 to 10:1, 1:6 to 10:1, 1:5 to 10:1, 1:4 to 10:1, 1:3 to 10:1, 1:2 to 10:1, 1:1 to 10:1, 1:10 to 9:1, 1:10 to 8:1, 1:10 to 7:1, 1:10 to 6:1, 1:10 to 5:1, 1:10 to 4:1, 1:10 to 3:1, 1:10 to 2:1, or 1:10 to 1:1, including ranges and subranges thereof. In one instance, the weight ratio of the total weight of compounds of Formula (I) to the total weight of another HAPH analog or metal chelate is 1:10 to 10:1, 1:9 to 10:1, 1:8 to 10:1, 1:7 to 10:1, 1:6 to 10:1, 1:5 to 10:1, 1:4 to 10:1, 1:3 to 10:1, 1:2 to 10:1, 1:1 to 10:1, 1:10 to 9:1, 1:10 to 8:1, 1:10 to 7:1, 1:10 to 6:1, 1:10 to 5:1, 1:10 to 4:1, 1:10 to 3:1, 1:10 to 2:1, or 1:10 to 1:1, including ranges and subranges thereof.

The amount of compounds of Formula (I) to Formula (VI) in the composition, may be a diagnostically effective amount. As used herein “diagnostically effective amount” refers to an amount that is effective to achieve a detecting limit in a living body. In some embodiments, the desired diagnostically effective amount may be an amount that sufficient to be detected in an imaging tool, including but not limited to PET, MRI, or CT. The amount of compound of Formula (I) through Formula (VI), such as metal chelates in an isotope form, may be more than about 1 μg. For example, the amount is at or about 2 μg or more, about 5 μg or more, about 10 μg or more, about 100 μg or more, about 500 μg or more, about 1000 μg or more, about 1500 μg or more, about 2000 μg or more, about 2500 μg or more, about 3000 μg or more, about 3500 μg or more, about 4000 μg or more, about 4500 μg or more, about 5000 μg or more, about 5500 μg or more, about 6000 μg or more, about 6500 μg or more, about 7000 μg or more, about 7500 μg or more, about 8000 μg or more, about 8500 μg or more, about 9000 μg or more, about 9500 μg or more, about 10 mg or more, about 20 mg or more.

The amount of compounds of Formula (I) to Formula (VI) in the composition, may be a diagnostically effective amount. As used herein “theranostically effective amount” refers to an amount that is sufficient to identify if target receptors are present on cancer cells, followed by precision radiation treatment that target these receptors. In some embodiments, the desired theranostically effective amount may be an amount that sufficient to be coupled with in an imaging tool, including but not limited to PET, MRI, or CT. The amount of compound of Formula (I) through Formula (VI), such as metal chelates in an isotope form, may be more than about 1 μg. For example, the amount is at or about 2 μg or more, about 5 μg or more, about 10 μg or more, about 100 μg or more, about 500 μg or more, about 1000 μg or more, about 1500 μg or more, about 2000 μg or more, about 2500 μg or more, about 3000 μg or more, about 3500 μg or more, about 4000 μg or more, about 4500 μg or more, about 5000 μg or more, about 5500 μg or more, about 6000 μg or more, about 6500 μg or more, about 7000 μg or more, about 7500 μg or more, about 8000 μg or more, about 8500 μg or more, about 9000 μg or more, about 9500 μg or more, about 10 mg or more, about 20 mg or more.

The HAPH composition can be formulated and administered to a subject by several different routes and means. For instance, a composition can generally be administered parenterally, intraperitoneally, intravascularly, topically, transdermally, subcutaneously, or intrapulmonarily in dosage unit formulations containing conventional nontoxic pharmaceutically or diagnositically acceptable adjuvants, carriers, excipients, and vehicles as desired. The term parenteral as used herein includes subcutaneous, intravenous, intramuscular, intrathecal, or intrasternal, or infusion techniques. In one aspect, therapeutically effective amount of HAPH analog/metal chelate can range from about 0.5 mcg to about 200 mg, about 1 mcg to about 180 mg, about 5 mcg to about 150 mg, or about 10 mcg to about 120 mg. The therapeutically effective amount of HAPH can range from about 0.5 mcg/day to about 100 mg/day, from about 1 mcg to about 60 mg/day, from about 20 mcg to about 50 mg/day, from about 20 mcg to about 30 mg/day, or from about 15 mcg to about 25 mg/day. Administering the HAPH composition may provide HAPH, or chelate at blood levels of at or about 1 ng/ml, at or about 2 ng/ml, at or about 4 ng/ml, at or about 5 ng/ml, at or about 6 ng/ml, at or about 8 ng/ml, at or about 10 ng/ml, at or about 12 ng/ml, at or about 15 ng/ml, at or about 20 ng/ml, at or about 22 ng/ml, at or about 24 ng/ml, at or about 26 ng/ml, at or about 28 ng/ml, at or about 30 ng/ml, at or about 32 ng/ml, at or about 34 ng/ml, at or about 36 ng/ml, at or about 38 ng/ml, at or about 40 ng/ml, at or about 42 ng/ml, at or about 44 ng/ml, 46 ng/ml, at or about 48 ng/ml, at or about 50 ng/ml.

A HAPH composition can be administered to the subject daily or more than once daily. For example, the composition can be administered to the subject once, twice or three times per day. The duration of each administration can vary from few seconds to about several hours, such as in infusion administration. Further, the HAPH composition can be administered every 2, 3, 4, 5, 6, 7, 14, or every 30 days. Such treatment regimen is subject to alter and adjust based on subject's individual needs and response sensitivity. The HAPH composition can be administered over a period ranging from about 1 day to about 1 year, from about 1 day to about 1 week, from about 3 days to about 1 month, from about 2 weeks to about 6 months, or from about 2 months to about 4 months. The HAPH composition can also be administered over a period of about 1 day, about 7 days, about 30 days, about 60 days, about 120 days, or about 180 days or more. In some aspect, A HAPH composition is administered over a period of about 57 weeks, about 148 weeks, about 208 weeks, indefinitely, or until resolution of the condition being treated.

A pharmaceutical formulation comprises one or more pharmaceutically acceptable excipients. Non-limiting examples of excipients include chemical enhancers, humectants, pressure sensitive adhesives, antioxidants, solubilizers, thickening agents, plasticizers, adjuvants, carriers, excipients, vehicles, coatings, and any combinations thereof. One or more excipients can be selected for oral, transdermal, parenteral, intraperitoneal, intravascular, subcutaneous, by inhalation spray, rectal, or intrapulmonary administration.

A HAPH composition can in general be formulated for improving patient compliance, preventing a subject from removing the drug-delivery device. For instance, HAPH composition could be formulated for improved patient compliance and preventing removal of a drug-delivery device by providing formulations for extended delivery. Extended delivery can range for periods ranging from more than one day, to months. This may be especially relevant for patients with compromised cognitive and/or motor-control abilities. Extended delivery for periods can range from about 1 day to about 1 year, from about 1 day to about 1 week, from about 3 days to about 1 month, from about 2 weeks to about 6 months, or from about 2 months to about 4 months.

Extended release formulations could be used for substantially continuous delivery of drug at a preselected rate. For example, for HAPH composition, the active can be delivered at a rate of from about 1 mg to about 100 mg/day, from about 40 to about 60 mg/day, or from about 10 to about 30 μm/day. Appropriate amounts of HAPH composition can be readily determined by the ordinarily skilled artisan based upon, for example, the intended duration of administration of the active by the extended release formulation, the delivery mechanism, the particular formulation, and the relative potency of the drug among other factors.

i. Binders

Non-limiting examples of binders suitable for the formulations of various aspects include starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohols, polyethylene glycol, polyols, saccharides, oligosaccharides, polypeptides, oligopeptides, and combinations thereof. The polypeptide may be any arrangement of amino acids ranging from about 100 to about 300,000 Daltons.

The binder can be introduced into the mixture to be granulated in a solid form, including but not limited to a crystal, a particle, a powder, or any other finely divided solid form known in the art. Alternatively, the binder can be dissolved or suspended in a solvent and sprayed onto the mixture in a granulation device as a binder fluid during granulation.

ii. Diluents

Non-limiting examples of diluents (also referred to as “fillers” or “thinners”) include carbohydrates, inorganic compounds, and biocompatible polymers, such as polyvinylpyrrolidone (PVP). Other non-limiting examples of diluents include dibasic calcium sulfate, tribasic calcium sulfate, starch, calcium carbonate, magnesium carbonate, microcrystalline cellulose, dibasic calcium phosphate, tribasic calcium phosphate, magnesium carbonate, magnesium oxide, calcium silicate, talc, modified starches, saccharides such as sucrose, dextrose, lactose, microcrystalline cellulose, fructose, xylitol, and sorbitol, polyhydric alcohols; starches; pre-manufactured direct compression diluents; and mixtures of any of the foregoing.

iii. Disintegrants

Disintegrants can be effervescent or non-effervescent. Non-limiting examples of non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth. Suitable effervescent disintegrants include but are not limited to sodium bicarbonate in combination with citric acid, and sodium bicarbonate in combination with tartaric acid.

iv. Preservatives

Non-limiting examples of preservatives include, but are not limited to, ascorbic acid and its salts, ascorbyl palmitate, ascorbyl stearate, anoxomer, N-acetylcysteine, benzyl isothiocyanate, m-aminobenzoic acid, o-aminobenzoic acid, p-aminobenzoic acid (PABA), butylated hydroxyanisole (BHA), butylated hydroxytoluene (BHT), caffeic acid, canthaxantin, alpha-carotene, beta-carotene, beta-caraotene, beta-apo-carotenoic acid, carnosol, carvacrol, catechins, cetyl gallate, chlorogenic acid, citric acid and its salts, clove extract, coffee bean extract, p-coumaric acid, 3,4-dihydroxybenzoic acid, N,N′-diphenyl-p-phenylenediamine (DPPD), dilauryl thiodipropionate, distearyl thiodipropionate, 2,6-di-tert-butylphenol, dodecyl gallate, edetic acid, ellagic acid, erythorbic acid, sodium erythorbate, esculetin, esculin, 6-ethoxy-1,2-dihydro-2,2,4-trimethylquinoline, ethyl gallate, ethyl maltol, ethylenediaminetetraacetic acid (EDTA), eucalyptus extract, eugenol, ferulic acid, flavonoids (e.g., catechin, epicatechin, epicatechin gallate, epigallocatechin (EGC), epigallocatechin gallate (EGCG), polyphenol epigallocatechin-3-gallate), flavones (e.g., apigenin, chrysin, luteolin), flavonols (e.g., datiscetin, myricetin, daemfero), flavanones, fraxetin, fumaric acid, gallic acid, gentian extract, gluconic acid, glycine, gum guaiacum, hesperetin, alpha-hydroxybenzyl phosphinic acid, hydroxycinammic acid, hydroxyglutaric acid, hydroquinone, N-hydroxysuccinic acid, hydroxytryrosol, hydroxyurea, rice bran extract, lactic acid and its salts, lecithin, lecithin citrate; R-alpha-lipoic acid, lutein, lycopene, malic acid, maltol, 5-methoxy tryptamine, methyl gallate, monoglyceride citrate; monoisopropyl citrate; morin, beta-naphthoflavone, nordihydroguaiaretic acid (NDGA), octyl gallate, oxalic acid, palmityl citrate, phenothiazine, phosphatidylcholine, phosphoric acid, phosphates, phytic acid, phytylubichromel, pimento extract, propyl gallate, polyphosphates, quercetin, trans-resveratrol, rosemary extract, rosmarinic acid, sage extract, sesamol, silymarin, sinapic acid, succinic acid, stearyl citrate, syringic acid, tartaric acid, thymol, tocopherols (i.e., alpha-, beta-, gamma- and delta-tocopherol), tocotrienols (i.e., alpha-, beta-, gamma- and delta-tocotrienols), tyrosol, 38hosphor acid, 2,6-di-tert-butyl-4-hydroxymethylphenol (i.e., Ionox 100), 2,4-(tris-3′,5′-bi-tert-butyl-4′-hydroxybenzyl)-mesitylene (i.e., Ionox 330), 2,4,5-trihydroxybutyrophenone, ubiquinone, tertiary butyl hydroquinone (TBHQ), thiodipropionic acid, trihydroxy butyrophenone, tryptamine, tyramine, uric acid, vitamin K and derivates, vitamin Q10, wheat germ oil, zeaxanthin, or combinations thereof.

v. Flavor-Modifying Agents

Suitable flavor-modifying agents include flavorants, taste-masking agents, sweeteners, and the like. Flavorants include, but are not limited to, synthetic flavor oils and flavoring aromatics and/or natural oils, extracts from plants, leaves, flowers, fruits, and combinations thereof. Other non-limiting examples of flavors include cinnamon oils, oil of wintergreen, peppermint oils, clover oil, hay oil, anise oil, eucalyptus, vanilla, citrus oils such as lemon oil, orange oil, grape and grapefruit oil, fruit essences including apple, peach, pear, strawberry, raspberry, cherry, plum, pineapple, and apricot.

Taste-masking agents include but are not limited to cellulose hydroxypropyl ethers (HPC) such as Klucel®, Nisswo HPC and PrimaFlo HP22; low-substituted hydroxypropyl ethers (L-HPC); cellulose hydroxypropyl methyl ethers (HPMC) such as Seppifilm-LC, Pharmacoat®, Metolose SR, Opadry YS, PrimaFlo, MP3295A, Benecel MP824, and Benecel MP843; methylcellulose polymers such as Methocel® and Metolose®; Ethylcelluloses (EC) and mixtures thereof such as E461, Ethocel®, Aqualon®-EC, Surelease; Polyvinyl alcohol (PVA) such as Opadry AMB; hydroxyethylcelluloses such as Natrosol®; carboxymethylcelluloses and salts of carboxymethylcelluloses (CMC) such as Aualon®-CMC; polyvinyl alcohol and polyethylene glycol co-polymers such as Kollicoat IR®; monoglycerides (Myverol), triglycerides (KLX), polyethylene glycols, modified food starch, acrylic polymers and mixtures of acrylic polymers with cellulose ethers such as Eudragit® EPO, Eudragit® RD100, and Eudragit® E100; cellulose acetate phthalate; sepifilms such as mixtures of HPMC and stearic acid, cyclodextrins, and mixtures of these materials. In other aspects, additional taste-masking agents contemplated are those described in U.S. Pat. Nos. 4,851,226; 5,075,114; and 5,876,759, each of which is hereby incorporated by reference in its entirety.

Non-limiting examples of sweeteners include glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia rebaudiana (Stevioside); chloro derivatives of sucrose such as sucralose; sugar alcohols such as sorbitol, mannitol, sylitol, hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

vi. Lubricants and Glidants

The lubricant compositions may be utilized to lubricate ingredients that form a pharmaceutical composition. As a glidant, the lubricant facilitates removal of solid dosage forms during the manufacturing process. Non-limiting examples of lubricants and glidants include magnesium stearate, calcium stearate, zinc stearate, hydrogenated vegetable oils, sterotex, polyoxyethylene monostearate, talc, polyethylene glycol, sodium benzoate, sodium lauryl sulfate, magnesium lauryl sulfate, and light mineral oil. The pharmaceutical composition will generally comprise from about 0.01% to about 10% by weight of a lubricant. In some aspects, the pharmaceutical composition will comprise from about 0.1% to about 5% by weight of a lubricant. In a further aspect, the pharmaceutical composition will comprise from about 0.5% to about 2% by weight of a lubricant.

vii. Dispersants

Dispersants may include but are not limited to starch, alginic acid, polyvinylpyrrolidones, guar gum, kaolin, bentonite, purified wood cellulose, sodium starch glycolate, isoamorphous silicate, and microcrystalline cellulose as high hydrophilic-lipophilic balance (HLB) emulsifier surfactants.

viii. Colorants

Depending upon the aspect of the disclosure, it may be desirable to include a coloring agent. Suitable color additives include but are not limited to food, drug and cosmetic colors (FD&C), drug and cosmetic colors (D&C), or external drug and cosmetic colors (Ext. D&C). These colors or dyes, along with their corresponding lakes, and certain natural and derived colorants, may be suitable for use in various aspects of the disclosure.

### ix. pH Modifiers

Non-limiting examples of pH modifiers include citric acid, acetic acid, tartaric acid, malic acid, fumaric acid, lactic acid, phosphoric acid, sorbic acid, benzoic acid, sodium carbonate and sodium bicarbonate.

x. Chelating Agents

A chelating agent may be included as an excipient to immobilize oxidative groups, including but not limited to metal ions, in order to inhibit the oxidative degradation of the morphinan by these oxidative groups. Non-limiting examples of chelating agents include lysine, methionine, glycine, gluconate, polysaccharides, glutamate, aspartate, and disodium ethylenediaminetetraacetate (Na₂EDTA).

xi. Antimicrobial Agents

An antimicrobial agent may be included as an excipient to minimize the degradation of the compound according to this disclosure by microbial agents, including but not limited to bacteria and fungi. Non-limiting examples of antimicrobials include parabens, chlorobutanol, phenol, calcium propionate, sodium nitrate, sodium nitrite, Na₂EDTA, and sulfites including but not limited to sulfur dioxide, sodium bisulfite, and potassium hydrogen sulfite.

xii. Release-Controlling Polymers

Release-controlling polymers may be included in the various aspects of the solid dosage pharmaceutical compositions incorporating compounds according to this disclosure. In one aspect, the release-controlling polymers may be used as a tablet coating. In other aspects, including but not limited to bilayer tablets, a release-controlling polymer may be mixed with the granules and other excipients prior to the formation of a tablet by a known process including but not limited to compression in a tablet mold. Suitable release-controlling polymers include but are not limited to hydrophilic polymers and hydrophobic polymers.

Suitable hydrophilic release-controlling polymers include, but are not limited to, cellulose acetate, cellulose diacetate, cellulose triacetate, cellulose ethers, hydroxyethyl cellulose, hydroxypropyl cellulose, hydroxypropyl methylcellulose, microcrystalline cellulose, nitrocellulose, crosslinked starch, agar, casein, chitin, collagen, gelatin, maltose, mannitol, maltodextrin, pectin, pullulan, sorbitol, xylitol, polysaccharides, ammonia alginate, sodium alginate, calcium alginate, potassium alginate, propylene glycol alginate, alginate sodium carmellose, calcium carmellose, carrageenan, fucoidan, furcellaran, 41hosph gum, carrageens gum, ghafti gum, guar gum, karaya gum, locust bean gum, okra gum, tragacanth gum, scleroglucan gum, xanthan gum, hypnea, laminaran, acrylic polymers, acrylate polymers, carboxyvinyl polymers, copolymers of maleic anhydride and styrene, copolymers of maleic anhydride and ethylene, copolymers of maleic anhydride propylene or copolymers of maleic anhydride isobutylene), crosslinked polyvinyl alcohol and poly N-vinyl-2-pyrrolidone, diesters of polyglucan, polyacrylamides, polyacrylic acid, polyamides, polyethylene glycols, polyethylene oxides, poly(hydroxyalkyl methacrylate), polyvinyl acetate, polyvinyl alcohol, polyvinyl chloride, polystyrenes, polyvinylpyrrolidone, anionic and cationic hydrogels, and combinations thereof.

xiii. Coatings

A solid dosage comprising a compound according to this disclosure may comprise a coating, wherein such a coating may control release of the compound, act as a moisture barrier, or buffer or modify pH. A “control releasing coat” or “controlled release coat” as used herein is defined to mean a functional coat which can for example comprise at least one pH independent polymer, pH dependent polymer (for example enteric or reverse enteric type polymers), soluble polymer, insoluble polymer, lipids, lipidic materials, or combinations thereof. The coating, when applied onto a dosage form, may slow (for example when applied to a normal release matrix dosage form), further slow (for example when applied to a controlled release matrix dosage form) or modify the rate of release of a compound according to this disclosure when applied to an uncoated dosage form. For example, the control releasing coat can be designed such that when the control releasing coat is applied to a dosage form, the dosage form in conjunction with the control releasing coat can exhibit the release of the compound according to this disclosure, such as a “modified-release”, “controlled-release”, “sustained-release”, “extended-release”, “delayed-release”, “prolonged-release,” or combinations thereof. The “control releasing coat” may optionally comprise additional materials that may alter the functionality of the control releasing coat.

The term “moisture barrier” as used herein is one which impedes or retards the absorption of moisture. Compounds according to this disclosure may be hygroscopic and, as such, may be susceptible to decomposition over time under highly humid conditions. The proportion of the components of the moisture barrier and the amount of the moisture barrier optionally applied onto the control-releasing coating or onto the core are typically such that the moisture barrier does not fall within the USP definition and requirement for an enteric coat. Suitably, the moisture barrier may comprise an enteric and/or acrylic polymer, suitably an acrylic polymer, optionally a plasticizer, and a permeation enhancer. The permeation enhancer is a hydrophilic substance, which allows water to enter without physical disruption of the coating. The moisture barrier may additionally comprise other conventional inert excipients, which may improve processing of an extended-release formulation.

Coating and matrix materials which may be used in accordance with the present disclosure are those known in the art for use in controlled-release formulations, such as synthetic polymers of the polyvinyl type, e.g., polyvinylchloride, polyvinylacetate and copolymers thereof, polyvinylalcohol, and polyvinylpyrrolidone; synthetic polymers of the polyethylene type, e.g., polyethylene and polystyrene; acrylic acid polymers; biopolymers or modified biopolymers, such as cellulosic polymers, shellac and gelatin; fats, oils, higher fatty acids and higher alcohols (i.e., acids and alcohols containing alkyl chains of at least 10 carbon atoms), for example aluminum monostearate, cetylalcohol, hydrogenated beef tallow, hydrogenated castor oil, 12-hydroxystearl alcohol, glyceryl mono- or dipalmitate; glyceryl mono-, di- or tristearate; myristyl alcohol, stearic acid, stearyl alcohol, and polyethyleneglycols; waxes; sugars and sugar alcohols.

The pH-buffering properties of a coating may be strengthened by introducing into the coating substances chosen from a group of compounds usually used in antacid formulations, for example magnesium oxide, hydroxide or carbonate, aluminum or calcium hydroxide, carbonate or silicate; composite aluminum/magnesium compounds, for example Al₂O₃·6MgO·CO₂·12H₂O, (Mg₆Al₂(OH)₁₆CO₃·4H₂O), MgO·Al₂O₃·2SiO₂·nH₂O, aluminum bicarbonate coprecipitate or similar compounds; or other pharmaceutically acceptable pH-buffering compounds, for example the sodium, potassium, calcium, magnesium and aluminum salts of phosphoric, carbonic, citric or other suitable, weak, inorganic or organic acids; or suitable organic bases, including basic amino acids; and salts or combinations thereof.

A pH-dependent coating serves to release the drug in desired areas of the gastrointestinal (GI) tract, e.g., the stomach or small intestine. When a pH-independent coating is desired, the coating is designed to achieve optimal release regardless of pH-changes in the environmental fluid, e.g., the GI tract. When the coating is formulated to release a compound according to this disclosure in the intestines (especially the upper small intestines), the coating is often called an “enteric coating”. A pH-dependent coating may include, but is not limited to, acrylic acid polymers and copolymers, for example polymers formed from acrylic acid, methacrylic acid, methyl acrylate, ammonio methylacrylate, ethyl acrylate, methyl methacrylate and/or ethyl methacrylate (e.g., Eudragit™); cellulosic polymers such as hydroxypropyl cellulose, hydroxyethyl cellulose, hydroxypropyl methyl cellulose, methyl cellulose, ethyl cellulose, cellulose acetate, cellulose acetate phthalate (CAP), cellulose acetate trimellitate, hydroxypropylmethyl cellulose phthalate, hydroxypropylmethyl cellulose succinate and carboxymethylcellulose sodium; shellac (purified lac); vinyl polymers and copolymers such as polyvinyl pyrrolidone, polyvinyl acetate, polyvinylacetate phthalate (PVAP), vinylacetate crotonic acid copolymer, and ethylene-vinyl acetate copolymers; zein; and salts and combinations thereof.

ix. Nanoparticles

The HAPH compositions may comprise nanoparticles wherein each of the nanoparticles comprises a carrier entity and at least one HAPH active, wherein the active is arranged on an outside surface of the nanoparticles and wherein the nanoparticles are capable of binding to a predetermined epitope in vivo. In another aspect, provided herein are nanoparticle compositions comprising nanoparticles, wherein each of the nanoparticles comprises at least one HAPH actives, wherein the HAPH active is arranged on a surface of the nanoparticles such that the binding with subject's receptors or cells is directed outward from that surface and wherein the nanoparticles are capable of binding to a predetermined epitope in vivo. In other embodiments, the nanoparticles multimerize, e.g. dimerize. Multimerization may be observed as multiples of the weight or size of the unit molecule, e.g. 160 nm particles multimerize to about 320 nm, 480 nm, 640 nm, etc. In some embodiments, less than 20% of the population are multimers. In some embodiments, more than 80% of the population are multimers.

x. Liposomes

In one alternative embodiment, a liposome delivery vehicle may be utilized. Liposomes, depending upon the embodiment, may be used for delivery of a HAPH composition comprising compounds of Formula (I) or Formula (II), or their metal chelates thereof, in view of their structural and chemical properties. Generally, liposomes are spherical vesicles with a phospholipid bilayer membrane. The lipid bilayer of a liposome may fuse with other bilayers (e.g., the cell membrane), thus delivering the contents of the liposome to cells.

Liposomes may be comprised of a variety of different types of phosolipids having varying hydrocarbon chain lengths. Phospholipids generally comprise two fatty acids linked through glycerol phosphate to one of a variety of polar groups. Suitable phospholids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unsaturated. Suitable fatty acid chains include (common name presented in parentheses)n-dodecanoate (laurate), n-tretradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecandienoate (linoleate), all cis-9,12,15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soybeans contain PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-deheptyloxacarbocyanine iodide, 1, T-dedodecyl-3,3,3′,3′-tetramethylindocarbocyanine perchloarate, 1, T-dioleyl-3,3,3′,3′-tetramethylindo carbocyanine methanesulfonate, N-4-(delinoleylaminostyryl)-N-methylpyridinium iodide, or 1,1,-dilinoleyl-3,3,3′,3′-tetramethylindocarbocyanine perchloarate.

Liposomes may optionally comprise sphingolipids, in which spingosine is the structural counterpart of glycerol and one of the one fatty acids of a phosphoglyceride, or cholesterol, a major component of animal cell membranes. Liposomes may optionally contain pegylated lipids, which are lipids covalently linked to polymers of polyethylene glycol (PEG). PEGs may range in size from about 500 to about 10,000 daltons.

Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetro nitrile, alcohols, dimethylformamide, tetrahydrofuran, or combinations thereof.

xi. Emulsions

The HAPH composition may be formulated as part of a microemulsion. Microemulsions are generally clear, thermodynamically stable solutions comprising an aqueous solution, a surfactant, and “oil.” The “oil” in this case, is the supercritical fluid phase. The surfactant rests at the oil-water interface. Any of a variety of surfactants are suitable for use in microemulsion formulations including those described herein or otherwise known in the art. The aqueous microdomains suitable for use according to the present disclosure generally will have characteristic structural dimensions from about 5 nm to about 100 nm. Aggregates of this size are poor scatterers of visible light and hence, these solutions are optically clear. As will be appreciated by a skilled artisan, microemulsions can and will have a multitude of different microscopic structures including sphere, rod, or disc shaped aggregates. In one embodiment, the structure may be micelles, which are the simplest microemulsion structures that are generally spherical or cylindrical objects. Micelles are like drops of oil in water, and reverse micelles are like drops of water in oil. In an alternative embodiment, the microemulsion structure is the lamellae. It comprises consecutive layers of water and oil separated by layers of surfactant. The “oil” of microemulsions optimally comprises phospholipids.

Any of the phospholipids detailed above for liposomes are suitable for embodiments directed to microemulsions. A composition comprising at least one anti-tumor therapeutic derivative may be encapsulated in a microemulsion by any method generally known in the art.

In yet another embodiment, the HAPH composition comprising compounds of Formula (I) or Formula (II), or metal chelates thereof may be delivered in a dendritic macromolecule, or a dendrimer. Generally, a dendrimer is a branched tree-like molecule, in which each branch is an interlinked chain of molecules that divides into two new branches (molecules) after a certain length. This branching continues until the branches (molecules) become so densely packed that the canopy forms a globe. Generally, the properties of dendrimers are determined by the functional groups at their surface. For example, hydrophilic end groups, such as carboxyl groups, would typically make a water-soluble dendrimer. Alternatively, phospholipids may be incorporated in the surface of a dendrimer to facilitate absorption across the skin. Any of the phospholipids detailed for use in liposome embodiments are suitable for use in dendrimer embodiments. Any method generally known in the art may be utilized to make dendrimers and to encapsulate compositions as disclosed herein. For example, dendrimers may be produced by an iterative sequence of reaction steps, in which each additional iteration leads to a higher order dendrimer. Consequently, they have a regular, highly branched 3D structure, with nearly uniform size and shape. Furthermore, the final size of a dendrimer is typically controlled by the number of iterative steps used during synthesis. A variety of dendrimer sizes are suitable for use according to the present disclosure. Generally, the size of dendrimers may range from about 1 nm to about 100 nm.

### VII. Dosage Forms

One aspect of the disclosure encompasses dosage forms made from the HAPH compositions. When the active is a HAPH analog, and/or a chelate, the dosage form may comprise the active from about 1 microgram to about 50 g, from about 1 mcg to about 5,000 mg, from about 1 mcg to about 1,000 mg, from about 1 mcg to about 500 mg, from about 50 mcg to about 400 mg, from about 75 mcg to about 150 mg, from about 150 mcg to about 200 mg, from about 40 mcg to about 150 mg, from about 80 mcg to about 120 mg, or from about 180 mcg to about 100 mg. For instance, the dosage form can comprise 1, 10, 20, 30, 40, 50, 60, 70, 80, 90, 100, 120, 140, 160, 180, 200, 220, 240, 260, 280, or 300 or more mcg of the HAPH active. In some aspect, dosage forms can comprise from about 1 mcg to about 500 mg, about 1 mcg to about 400 mg, about 1 mcg to about 300 mg, about 1 mcg to about 250 mg, about 1 mcg to about 200 mg, about 1 mcg to about 150 mg, or about 1 mcg to about 100 mg of the HAPH active.

Dosage forms include those formulated for extended or slow release, and those formulated for immediate release. For example, an immediate release dosage form may include a crystalline form of HAPH analog as the free base or as a HAPH salt as disclosed herein. For example, a fast-dissolve oral dosage form may include for example an HAPH salt, such as the HCl salt. Alternatively, a dosage form may include a crystalline form of HAPH analog as the free base or as a HAPH salt crystalline form.

Dosage forms also include those formulated for topical administration. For instance, a dosage form can be formulated as one or more of a gel, ointment, emulsion, microemulsion, lipids, liposomes, nanosomes, solution, suspension, paste, gel, foam, spray, lotion, or cream. In one aspect, a topical administration dosage form is a transdermal patch. When the dosage form is formulated as a transdermal patch, the transdermal patch can contain from at or about 40 mcg to at or about 60 mg, from at or about 80 mcg to at or about 50 mg, or from about 180 mcg to at or about 40 mg of HAPH freebase in crystalline form.

Dosage forms can alternatively be formulated for oral administration. Dosage forms formulated for oral administration can be tablets to swallow, chew, or dissolve in water or under the tongue, capsules and chewable capsules, powders, granules, teas, drops, or liquid medications or syrups. In some aspect, the dosage form is an enteric coated oral formulation.

When the dosage form is an enteric coated oral formulation, the formulation can comprise from about 0.1 mcg to about 60 mg HAPH analog, preferably from about 1 mcg to about 50 mg HAPH analog.

An enteric coated oral formulation can also contain HAPH analog salt in a crystalline form. The salt of a HAPH analog can be a fumarate salt, a sulfate salt, a mesylate salt, a dihydrogen phosphate salt, an edisylate salt, a benzoate salt, a hydrochloride salt, and an oxalate salt. In one aspect, the salt of the HAPH analog is a fumarate salt. When the salt is a fumarate salt, the enteric coated oral formulation can comprise from about 0.1 to about 100 mg of fumarate salt of the HAPH analog, preferably from about 1 mg to about 55 mg of fumarate salt of the HAPH analog. In another aspect, the salt of the HAPH analog is a hydrochloride salt. When the salt is a hydrochloride salt, the enteric coated oral formulation can comprise from about 0.1 to about 200 mg of hydrochloride salt of the HAPH analog, and preferably from about 1 mg to about 100 mg of hydrochloride salt of the HAPH analog.

Dosage forms also encompass those formulated for subcutaneous and/or intramuscular injection. For example, an intramuscular dosage form may comprise HAPH analog in crystal form, dissolved in an oil matrix for intramuscular injection, or alternatively prepared as a suspension of the free base for intramuscular injection. A dosage form formulated for subcutaneous or intramuscular injection may comprise HAPH analog in a salt or crystal form as disclosed herein, prepared as microspheres using methods known in the art. Alternatively, HAPH analog or salt form may be coated, for example using Atomic Layer Deposition (ALD) techniques, with a thin layer coating such as a coating of zinc oxide, and used in a formulation for subcutaneous or intramuscular injection. Alternatively, HAPH analog may be dissolved in a biodegradable polymer matrix, and then implanted subcutaneously (or used in a transdermal patch as detailed further below).

### VIII. Kits

A further aspect of the present disclosure provides kits comprising one or more compositions for use according to the present disclosure. The kits may comprise a container to hold the HAPH composition and an instruction of use. The kits generally include instructions for carrying out the methods detailed in the present disclosure. Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides such instructions.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2^{nd} ed. 1994); The Cambridge Dictionary of Science and Technology (Walker ed., 1988); The Glossary of Genetics, 5^{th} Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “comprising” means “including, but not necessarily limited to”; it specifically indicates open-ended inclusion or membership in a so-described combination, group, series and the like. The terms “comprising” and “including” as used herein are inclusive and/or open-ended and do not exclude additional, unrecited elements or method processes. The term “consisting essentially of” is more limiting than “comprising” but not as restrictive as “consisting of.” Specifically, the term “consisting essentially of” limits membership to the specified materials or steps and those that do not materially affect the essential characteristics of the claimed invention.

The term “alkyl,” as used herein, describes groups that are preferably lower alkyl containing from one to eight carbon atoms in the principal chain and up to 20 carbon atoms. They may be straight or branched chain or cyclic and include methyl, ethyl, propyl, isopropyl, butyl, hexyl and the like.

The term “alkenyl,” as used herein, describes groups that are preferably lower alkenyl containing from two to eight carbon atoms in the principal chain and up to 20 carbon atoms. They may be straight or branched chain or cyclic and include ethenyl, propenyl, isopropenyl, butenyl, isobutenyl, hexenyl, and the like.

The term “alkynyl,” as used herein, describes groups that are preferably lower alkynyl containing from two to eight carbon atoms in the principal chain and up to 20 carbon atoms. They may be straight or branched chain and include ethynyl, propynyl, butynyl, isobutynyl, hexynyl, and the like.

The term “aromatic,” as used herein, alone or as part of another group denotes optionally substituted homo- or heterocyclic planar ring or ring system comprising delocalized electrons. These aromatic groups are preferably monocyclic (e.g., furan or benzene), bicyclic, or tricyclic groups containing from 5 to 14 atoms in the ring portion. The term “aromatic” encompasses “aryl” groups defined below.

The terms “aryl” or “Ar,” as used herein, alone or as part of another group denote optionally substituted homocyclic aromatic groups, preferably monocyclic or bicyclic groups containing from 6 to 10 carbons in the ring portion, such as phenyl, biphenyl, naphthyl, substituted phenyl, substituted biphenyl, or substituted naphthyl.

The terms “carbocyclo” or “carbocyclic,” as used herein, alone or as part of another group denote optionally substituted, aromatic or non-aromatic, homocyclic ring or ring system in which all of the atoms in the ring are carbon, with preferably 5 or 6 carbon atoms in each ring. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxy, keto, ketal, 51hosphor, nitro, and thio.

The terms “halogen” or “halo,” as used herein, alone or as part of another group refer to chlorine, bromine, fluorine, and iodine.

The term “heteroatom,” as used herein, refers to atoms other than carbon and hydrogen. The term “heteroaromatic,” as used herein, alone or as part of another group denotes optionally substituted aromatic groups having at least one heteroatom in at least one ring, and preferably 5 or 6 atoms in each ring. The heteroaromatic group may have 1 to 4 oxygen, nitrogen, silicone, and/or sulfater atoms (e.g. 1 or 2 oxygen atoms and/or 1 to 4 nitrogen atoms) in the ring, and is bonded to the remainder of the molecule through a carbon. Exemplary groups include fury I, benzofuryl, oxazolyl, isoxazolyl, oxadiazolyl, benzoxazolyl, benzoxadiazolyl, pyrrolyl, pyrazolyl, imidazolyl, triazolyl, tetrazolyl, pyridyl, pyrimidyl, pyrazinyl, pyridazinyl, indolyl, isoindolyl, indolizinyl, benzimidazolyl, indazolyl, benzotriazolyl, tetrazolopyridazinyl, carbazolyl, purinyl, quinolinyl, isoquinolinyl, imidazopyridyl, and the like. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxy, keto, ketal, 52hosphor, nitro, and thio.

The terms “heterocyclo” or “heterocyclic,” as used herein, alone or as part of another group denote optionally substituted, fully saturated or unsaturated, monocyclic or bicyclic, aromatic or non-aromatic groups having at least one heteroatom in at least one ring, and preferably 5 or 6 atoms in each ring. The heterocyclo group may have 1 or 2 oxygen atoms and/or 1 to 4 nitrogen atoms in the ring, and is bonded to the remainder of the molecule through a carbon or heteroatom. Exemplary heterocyclo groups include heteroaromatics as described above. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxy, keto, ketal, 52hosphor, nitro, and thio.

The terms “hydrocarbon” and “hydrocarbyl,” as used herein, refer to organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

The compounds described herein may have asymmetric centers. Compounds of the present disclosure containing an asymmetrically substituted atom may be isolated in optically active or racemic form. All chiral, diastereomeric, racemic forms and all geometric isomeric forms of a structure are intended, unless the specific stereochemistry or isomeric form is specifically indicated.

As used herein, the terms “disease”, “disorder” or “medical condition” are used interchangeably. Each includes, but is not limited to, any condition or disease manifested as one or more physical and/or psychological symptoms for which treatment is desirable, and includes previously and newly identified diseases and other disorders.

As used herein, the term “anticancer”, “anti-tumor” and “antineoplastic” may be used interchangeably, for an activity in combating cancer or tumor. Classes of anticancer agents include, but are not limited to, chemotherapeutic agents, cytotoxins, antimetabolites, alkylating agents, protein kinase inhibitors, anthracyclines, antibiotics, antimitotic agents (e.g. antitubulin agents), corticosteroids, radiopharmaceuticals, and proteins (e.g. cytokines, enzymes, or interferons). Specific examples include, but are not limited to docetaxel, gemcitabine, imatinib (Gleevec®), 5-fluorouracil, 9-aminocamptothecin, amine-modified geldanamycin, doxorubicin, paclitaxel (Taxol®), procarbazine, hydroxyurea, meso e-chlorin, cisplatin, Gd(+3) compounds, asparaginase, and radionuclides (e.g I-131, Y-90, In-111, and Tc-99m). There are many anticancer agents known in the art and many continue to be developed.

The terms “treat,” “treating,” and “treatment” are meant to include alleviating or abrogating a condition, or one or more of the symptoms associated with the condition; or alleviating or eradicating the cause(s) of the condition itself.

The terms “manage,” “managing,” and “management” encompass preventing the recurrence of the specified disease, disorder, or condition in a patient who has already suffered from the disease, disorder, or condition, and/or lengthening the time that a patient who has suffered from the disease, disorder, or condition remains in remission. The terms encompass modulating the threshold, development and/or duration of the disease, disorder, or condition, or changing the way that a patient responds to the disease, disorder, or condition.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition

As used herein, the term “effective amount” refers to an amount of an agent (such as a mixture of RNAs) that provides a desired biological, therapeutic, preventive, theranostic, and/or prophylactic result. That result can be reduction, amelioration, palliation, lessening, delaying, prevention, and/or alleviation of one or more of the signs, symptoms, or causes of a disease (such as advanced stage solid tumor cancer). In some embodiments, an effective amount comprises an amount sufficient to cause a solid tumor/lesion to shrink. In some embodiments, an effective amount is an amount sufficient to decrease the growth rate of a solid tumor (such as to suppress tumor growth). In some embodiments, an effective amount is an amount sufficient to delay tumor development. In some embodiments, an effective amount is an amount sufficient to prevent or delay tumor recurrence.

In some aspect, an effective amount is an amount sufficient to increase a subject's immune response to a tumor or a cancer, such that tumor growth and/or size and/or metastasis is reduced, delayed, ameliorated, and/or prevented. An effective amount can be administered in one or more administrations. In some embodiments, administration of an effective amount (e.g., of a composition comprising mRNAs) may: (i) reduce the number of cancer cells; (ii) reduce tumor size; (iii) inhibit, retard, slow to some extent and may stop cancer cell infiltration into peripheral organs; (iv) inhibit (e.g., slow to some extent and/or block or prevent) metastasis; (v) inhibit tumor growth; (vi) prevent or delay occurrence and/or recurrence of tumor; and/or (vii) relieve to some extent one or more of the symptoms associated with the cancer.

The term “co-administered” or “co-administration” or the like as used herein refers to administration of two or more agents concurrently, simultaneously, or essentially at the same time, either as part of a single formulation or as multiple formulations that are administered by the same or different routes. “Essentially at the same time” as used herein means within about 1 minute, 5 minutes, 10 minutes, 15 minutes, 30 minutes, 1 hour, 2 hours, or 6 hours period of each other.

As used herein, the term “subject” means that preferably the subject is a mammal, such as a human, but can also be an animal, e.g., domestic animals (e.g., dogs, cats and the like), farm animals (e.g., cows, sheep, pigs, horses and the like) and laboratory animals (e.g., cynomolgus monkey, rats, mice, guinea pigs and the like).

As used herein, the administration of an agent or drug to a subject or patient includes self-administration and the administration by another. It is also to be appreciated that the various modes of treatment or prevention of medical conditions as described are intended to mean “substantial”, which includes total but also less than total treatment or prevention, and wherein some biologically or medically relevant result is achieved.

### Examples

The publications discussed above are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. Study Design

At least one of the goals of the present disclosure is to synthesize novel HAPH analogs as exemplified by Formula (I)-(IV), and metal chelates thereupon. Another goal is to investigate how these HAPH analogs impact the oxygen-sensitivity of a biological system. The oxygen-sensitivity may manifest, for example, as DNA cleavage and/or DNA scissions. The oxygen-sensitivity may also be manifested as radical-formation resulting from oxygen activation. These oxygen-sensitivities may be led to various therapeutic uses, including but not limited to anti-tumor or anti-cancer treatments. Some exemplary HAPH analogs are listed in TABLE 1 and in FIGS. 1A-D. Some exemplary synthesis processes are given in FIGS. 2A-2G. Also contemplated are their metal chelates with various metal ions.

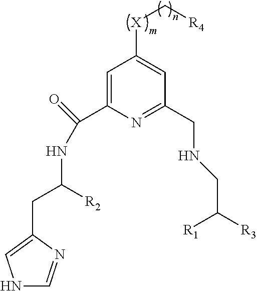

TABLE 1
HAPH ANALOGS

| NAME | R₁ | R₂ | R₃ | R₄ | X | m | n |
|---|---|---|---|---|---|---|---|
| HAPH-2 | 5-imidazolyl | H | H | NH₂ | N/A | 0 | 1 |
| HAPH-2 | 5-imidazolyl | H | H | NH₂ HCl | N/A | 0 | 1 |
| HCl Salt | | | | | | | |
| HAPH-2 | 5-imidazolyl | H | H | N₃ | CH₂—NHC(O) | 0 | 11 |
| Azide | | | | | | | |
| HAPH-2 | 5-imidazolyl | H | H | CΞC | CH₂—NHC(O) | 0 | 11 |
| Ethyne | | | | | | | |
| HAPH-2 | 5-imidazolyl | H | H | N₃ | N/A | 0 | 1 |
| Methylene | | | | | | | |
| Azide | | | | | | | |
| HAPH-3 | 5-imidazolyl | H | H | OH | N/A | 0 | 1 |
| HAPH-4 | 5-imidazolyl | H | H | COOH | N/A | 0 | 2 |
| HAPH-5 | 5-imidazolyl | H | H | COOH | N/A | 0 | 3 |
| HAPH-6 | 5-imidazolyl | H | H | NH₂ | N/A | 0 | 2 |
| HAPH-7 | 5-imidazolyl | H | H | NH₂ | N/A | 0 | 3 |
| HAPH-8 | 5-imidazolyl | H | H | OH | N/A | 0 | 2 |
| HAPH-9 | 5-imidazolyl | H | H | OH | N/A | 0 | 3 |
| HAPH-10 | 5-imidazolyl | H | H | NH₂ | O | 1 | 2 |
| HAPH-11 | 5-imidazolyl | H | H | COOH | O | 1 | 2 |
| HAPH-12 | 5-imidazolyl | H | H | OH | O | 1 | 2 |
| HAPH-13 | 5-imidazolyl | H | H | COOH | N/A | 0 | 1 |
| AMPHIS-1N | —NH₂ | —C(O)OCH₃ | H | NH₂ | N/A | 0 | 1 |
| AMPHIS-1O | —NH₂ | —C(O)OCH₃ | H | OH | N/A | 0 | 1 |
| AMPHIS-1A | —NH₂ | —C(O)OCH₃ | H | COOH | N/A | 0 | 1 |
| PYML-1N | NH₂ | —C(O)OH | —C(O)NH₂ | NH₂ | N/A | 0 | 1 |
| PYML-1O | NH₂ | —C(O)OH | —C(O)NH₂ | OH | N/A | 0 | 1 |
| PYML-1A | NH₂ | —C(O)OH | —C(O)NH₂ | COOH | N/A | 0 | 1 |
| SAPH-1N | —SMe | H | H | NH₂ | N/A | 0 | 1 |
| SAPH-1O | —SMe | H | H | OH | N/A | 0 | 1 |
| SAPH-1A | —SMe | H | H | COOH | N/A | 0 | 1 |
| SAPH-2N | —SCH₂C₆H₄OMe | H | H | NH₂ | N/A | 0 | 1 |
| SAPH-2O | —SCH₂C₆H₄OMe | H | H | OH | N/A | 0 | 1 |
| SAPH-2A | —SCH₂C₆H₄OMe | H | H | COOH | N/A | 0 | 1 |
| SAPH-3N | —SH | H | H | NH₂ | N/A | 0 | 1 |
| SAPH-3O | —SH | H | H | OH | N/A | 0 | 1 |
| SAPH-3A | —SH | H | H | COOH | N/A | 0 | 1 |

### Example 2. Physiochemical and Biological Testing

In order to assess the potential or efficacy of HAPH analogs and their chelates as DNA cleaving or scission agents, various physiochemical testing and biological assays are to be performed.

### Example 2A: Physiochemical Testing

HAPH analogs or its metal chelates are evaluated for their susceptibility to oxidation. Their solutions are adjusted to the desired pH by adding concentrated, Ar-purged NaOH, by means of a 1.00-mL calibrated, gastight syringe. Aliquots are withdrawn by syringes and transferred to 1.00- or 2.00-cm Ar-purged quartz cells for UV—visible spectrophotometry or quartz flat cells after oxidation by O₂ or H₂O₂ for the ESR spin-trapping studies. ESR Spectra is obtained using frozen-solution spectra12 at 77 K and recorded at 9.019 GHz, 1.60-G modulation amplitude, and 20.0-mW power with 8.0-min scans and a 1.0-s time constant. Receiver gains (RG) are measured. Samples, prepared as described above, are kept in liquid N2 inside of a quartz Dewar held in such a manner as to suspend the tube at the proper position in the microwave cavity of a Varian E-4 ESR spectrometer. The low-temperature ESR spectrum of the testing sample can be obtained in the same frozen glass in a quartz ESR tube mounted in a Varian temperature controller unit within the cavity. Scanning parameters may be set at 9.058-GHz microwave frequency, 6.3-G modulation amplitude, 20.0-mW power, 4.0-min scan, and 1.0-s time constant. The field is calibrated for all runs at room temperature with DPPH as the standard. Results are recorded and graphed as ESR spectrum.

In studies involving CO complex formation, the Ar purging gas is temporarily replaced by bubbling CO (Air Products) through the Cr″ gas train. The entire assembly for Ar or CO purging is carried out in a hood with a strong exhaust system to remove the vented CO gas. Reversing the coordination of CO was carried out by vigorous Ar flushing for a period of 15 min. Total recovery of the initial analogs or chelates may be confirmed by matching initial and final visible spectra. Redox potentials were measured with an IBM EC/225 voltammetric analyzer in the differential pulse (DP) and cyclic voltammetry (CV) modes. A pulse amplitude of 50 mV at a sweep rate of 10 mV/s is employed for DP; scan rates of 100 mV/s are typical for CV data. A three-electrode system consisting of a glassy-carbon working electrode, a sodium chloride saturated calomel reference electrode (SSCE), and a Pt-wire auxiliary electrode are used. The potential range was scanned immediately and then scanned at appropriate subsequent intervals up to 150 min.

Reversible CO binding with HAPH analogs and their chelates are studied as well. CO adducts with HAPH analogs or their chelates are prepared in bubblers and transferred to the N2-purged electrochemical cell by standard syringe techniques. A N2 atmosphere is maintained over the solution while the differential pulse polarograph is recorded. Bubbling these solutions with N₂ for—10 min allows the decay of the CO complex to be studied. A similar experiment is performed with CV. Reversibility is studied through an N2 purge, which allows recording of a CV trace matching the one prior to CO admission to the cell.

### Example 2B: Bioassays for DNA Cleavage

For example, the HAPH analogs shown in Table 1 and their metal chelates are tested by incubating the analogs, and/or chelates at various concentrations, with supercoiled plasmid DNA or linear DNA and assessing the cleavage using isolated fragments. Cleavage assays are conducted to determine if a potential drug targets gyrase. Some drugs interrupt the DNA breakage-reunion step of the gyrase reaction. This leads to cell death and it is the mechanism behind the action of many antitumor drugs. The cleavage activities of the HAPH analogs and metal chelates may be assessed using fluorescently labeled or radiolabeled DNA. Further, these studies can be extended to cell-based assays using various established cancer cell lines, such as BSMZ, a breast cancer cell line, LNCaP, a prostate cancer cell line, K-562, a human leukemia cell line; and Lewis Lung Carcinoma, a murine lung carcinoma cell line. DNA cleaving activities of the HAPH analogs are assessed by measuring changes in cellular characteristics and/or morphology at pre-specified endpoints after treatment. Measured determinants of cellular changes in characteristics and/or morphology may include analysis of cell proliferation, cell size, and cell senescence. Additionally, and alternatively, the inducement of oxygen radical formation is to be tested.

Another testing method involves use of topoisomerase. In a Topoisomerase DNA cleavage Assay, type II topoisomerases are used. These topoisomerases change DNA topology by cleavage and religation of both DNA strands. When an inhibitor such as fluoroquinolone drugs blocks the religation step of the topoisomerase reaction, the cleaved or linearized DNA forms covalent complexes with the enzyme (cleavage complex). Formation of DNA cleavage complexes causes DNA damage in the cells (topoisomerase poison). Testing formation of DNA cleavage complexes is therefore measured and calculated for cleavage activities.

### Example 2C: Bioassays for DNA Scission

DNA scission may occur during processes of DNA repair, programmed cell death (apoptosis), and necrosis. DNA scission of pathological cells may be viewed as a marker of therapeutic effects. DNA scission or fragmentation may be detected through incorporating treated cell tissue with TdT, DNA polymerase I, and the Klenow fragment of DNA polymerase. For the TdT experimentation, tissue sections treated with HAPH analogs or metal chelates are incubated with TdT (such as 0.3 U/μL) and digoxigenin-11-dUTP (such as 0.5 nmol/L) in 0.1 mol/L sodium cacodylate, 1 mmol/L CoCl2, 0.1 mmol/L dithiothreitol, and 50 μg/mL BSA for 60 minutes at 37° C. For the DNA polymerase I and the Klenow enzyme, adjacent tissue sections are incubated with a mixture of digoxigenin-conjugated dUTP and unlabeled deoxynucleotides, buffer containing 50 mmol/L Tris-HCl (pH 7.5), 10 mmol/L MgSO4, 0.1 mmol/L dithiothreitol, 50 μg/mL BSA, and the polymerase for 2 hours at 37° C. After treated sections are washed, incubation for 60 minutes with horseradish peroxidase-conjugated anti-digoxigenin antibody (Boehringer Mannheim) is performed to detect the incorporation of digoxigenin-conjugated dUTP. The chromogen 3-amino-9-ethylcarbazole (Biomeda Corp) is used for color development. Controls for the TdT and for polymerase-based procedures included (1) exposure of parallel sections to DNase I (Sigma) (positive control) and (2) deletion of the respective enzyme in each experiment (negative control for nonspecific staining) in situ. Comparing these methods, the TdT technique, in its most simplistic interpretation, is an indicator of significant cell death. DNA polymerase I holoenzyme exonuclease activity mediates nick translation and therefore allows visualization of randomly occurring single-strand scission of double-strand DNA. Both methods can be applied here for testing.

### Example 3. Synthesis of HAPH

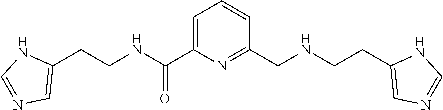 [SMILES: O=C(NCCc1cnc[nH]1)c1cccc(CNCCc2cnc[nH]2)n1]

HAPH was successfully synthesized. Briefly, histamine (0.22 g, 1.98 mmol) was dissolved in methanol (10 mL). Two drops of the indicator bromocresol purple (1% in methanol) was added to this solution. The solution was deep blue, indicating a basic pH. The pH was adjusted to 6 with the addition of 5 M HCl in methanol, as indicated by a yellow-green color. Size 3A molecular sieves were added to the flask. Methyl-2-formylpyridine-6-carboxylate (0.30 g, 1.8 mmol) was then added to the mixture. The solution was stirred for 1.5 h with the pH maintained at 6. Sodium cyanoborohydride (0.09 g, 0.95 mmol) was then added, and the solution was stirred for 2 days. An additional amount of histamine (0.66 g, 5.9 mmol) was then added to the flask. The solution was dark blue, indicating a basic pH, which was not altered. After it was stirred for an additional 4 days, the solution was filtered and concentrated under vacuum to 1 mL. The reaction mixture was purified by preparative TLC (CHCl3/C2H5OH/C2H5NH2 (70% aqueous solution), 80:20:2) to yield HAPH (0.365 g, 60% yield) as an oil.

### Example 4. Synthesis of HAPH-2

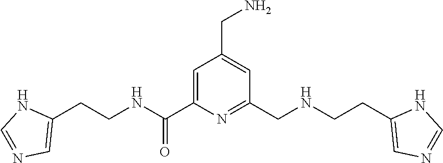 [SMILES: NCc1cc(CNCCc2cnc[nH]2)nc(C(=O)NCCc2cnc[nH]2)c1]

HAPH-2 was synthesized according to Scheme 2. Briefly, 6-Formyl-4-[(4-methoxy-benzylamino)-methyl]-pyridine-2-carboxylic acid methyl ester (0.5 g, 1.59 mmol) and histamine (0.212 g, 1.90 mmol) was taken in Hexafluorolsopropanol (5.0 ml) and stirred for 6 hours. Then Sodium borohydride (37 mg, 1.08 mmol) was added and stirred for 16 hours at room temperature. After completion of starting material in TLC, it was evaporated under reduced pressure to dryness. The crude was purified by column chromatography (Eluent: 25-30% MeOH in MDC) to afford 550 mg of 6-{[2-(3H-Imidazol-4-yl)-ethylamino]-methyl}-4-[(4-methoxy-benzylamino)-methyl]-pyridine-2-carboxylic acid methyl ester. Details of Step-1 through Step-3 were provided below.

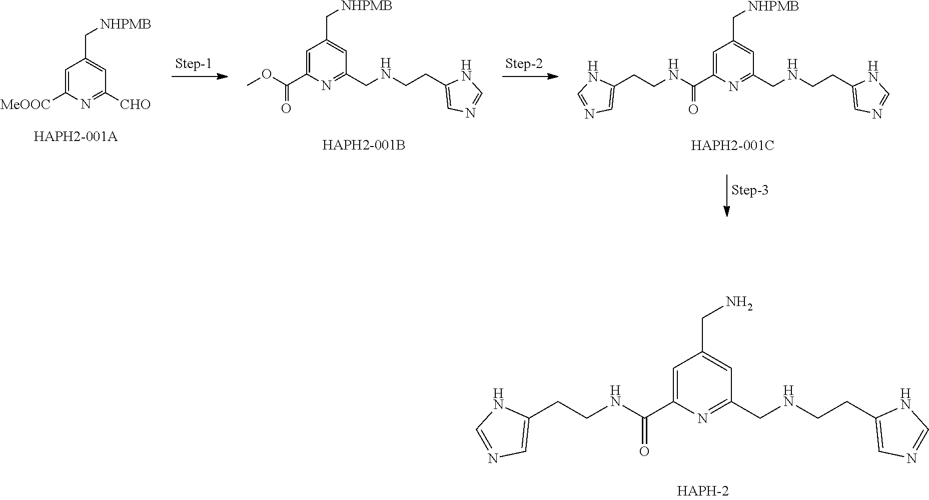

Step-1:6-Formyl-4-[(4-methoxy-benzylamino)-methyl]-pyridine-2-carboxylic acid methyl ester (0.5 g, 1.59 mmol) and histamine (0.212 g, 1.90 mmol) was taken in Hexafluorolsopropanol (5.0 ml) and stirred for 6 hours. Then Sodium borohydride (37 mg, 1.08 mmol) was added and stirred for 16 hours at room temperature. After completion of starting material in TLC, it was evaporated under reduced pressure to dryness. The crude was purified by column chromatography (Eluent: 25-30% MeOH in MDC) to afford 550 mg of 6-{[2-(3H-Imidazol-4-yl)-ethylamino]-methyl}-4-[(4-methoxy-benzylamino)-methyl]-pyridine-2-carboxylic acid methyl ester.

Step-2: To a stirred solution of 6-{[2-(3H-Imidazol-4-yl)-ethylamino]-methyl}-4-[(4-methoxy-benzylamino)-methyl]-pyridine-2-carboxylic acid methyl ester (0.55 g, 1.34 mmol) in MeOH (5 ml) histamine (447 mg, 4.02 mmol) was added at RT and stirred for 48 hours. After completion of starting material in TLC. It was evaporated under reduced pressure. The Crude compound was purified by column chromatography (Eluent: 30% MeOH in MDC) to afford 0.65 g of 6-{[2-(3H-Imidazol-4-yl)-ethylamino]-methyl}-4-[(4-methoxy-benzylamino)-methyl]-pyridine-2-carboxylic acid [2-(3H-imidazol-4-yl)-ethyl]-amide.

Step-3:6-{[2-(3H-Imidazol-4-yl)-ethylamino]-methyl}-4-[(4-methoxy-benzylamino)-methyl]-pyridine-2-carboxylic acid [2-(3H-imidazol-4-yl)-ethyl]-amide (0.65 g, 1.33 mmol) was taken in TFA (6 ml) and heated to 950° C. for 48 hrs, Crude LCMS shows 30% of product formation. Then the reaction mass was evaporated to dryness and purified by Prep HPLC to afford 4-Aminomethyl-6-{[2-(3H-imidazol-4-yl)-ethylamino]-methyl}-pyridine-2-carboxylic acid [2-(3H-imidazol-4-yl)-ethyl]-amide.

HAPH-2 was characterized by ¹H NMR, Mass Spectroscopy (MS), LCMS, and HPLC as shown in FIGS. 3A-3G. Thin layer chromatography (TLC) and PREP purification process were summarized as below.

TLC was performed in the following mobile phases. Step-1: Mobile phase 5% MeOH in MDC with 0.2 Rf. Step-2: Mobile phase 10% MeOH in MDC with 0.2 Rf, two times elution; and Step-3: Mobile phase 20% MeOH in MDC with 0.1 Rf, two times elution. Prep Conditions Included Trials with Mini Prep Conditions A-D. Mini Prep Condition A Had Gradient Program: Time/Pump B: 0/5,4/5, 16/25, 17/100,22/100,22.5/5,28/5; Flow Rate: 15 ml/Min; Column: X-Select C18 (*250*10 μm); Buffers Used Included Pump A: 10 mm Abc In Water And Pump B: Acetonitrile. Mini Prep Conditions B Had A Gradient Program With Time/Pump B: 0/5,4/5, 16/25, 17/100,22/100,22.5/5,28/5; Flow Rate: 15 ml/Min; Column: X-Bridge C18 (*250*10 μm); Buffers Used Included Pump A: 0.1% Tfa In Water And Pump B: Acetonitrile. Mini Prep Conditions C Had Gradient Program: Time/Pump B: 0/5,4/5, 16/25, 17/100,22/100,22.5/5,28/5; Flow Rate: 15 ml/Min; Column: X-Select (*250*10 μm); Buffers Used: Pump A: 0.1% TFA in Water And Pump B: Acetonitrile. Mini Prep Conditions D Included Gradient Program: Time/Pump B: 0/5,4/5, 16/25, 17/100,22/100,22.5/5,28/5; Flow Rate: 15 ml/Min; Column: X-Select (*250*10 μm) Dacc; Buffers Used included PUMP A: 0.1% TFA IN WATER and PUMP B: ACETONITRILE. Finalized conditions were summarized in TABLE 2.

TABLE 2
HAPH-2 Purification Details
Purification details

| | | Gradient | | |
|---|---|---|---|---|
| Compound | | table | | |
| ID | HAPH-II | TIME(min) | % A | % B |
| column ID | XSELECT CSH C18 | 0 | 95 | 5 |
| | (250*21 m*10 u) | | | |
| Flow rate | 15 ml/min | 2 | 95 | 5 |
| Mobile phase | | 16 | 75 | 25 |
| Phase A | 0.1% TFA acid in water | 19 | 5 | 95 |
| Phase B | HPLC grade Acetonitrile | 24 | 5 | 95 |
| Gradient: | Method in the table | 24.5 | 95 | 5 |
| | | 28 | 95 | 5 |

Retention time - 28 Min

### Example 5. Synthesis of HAPH-2 HCl

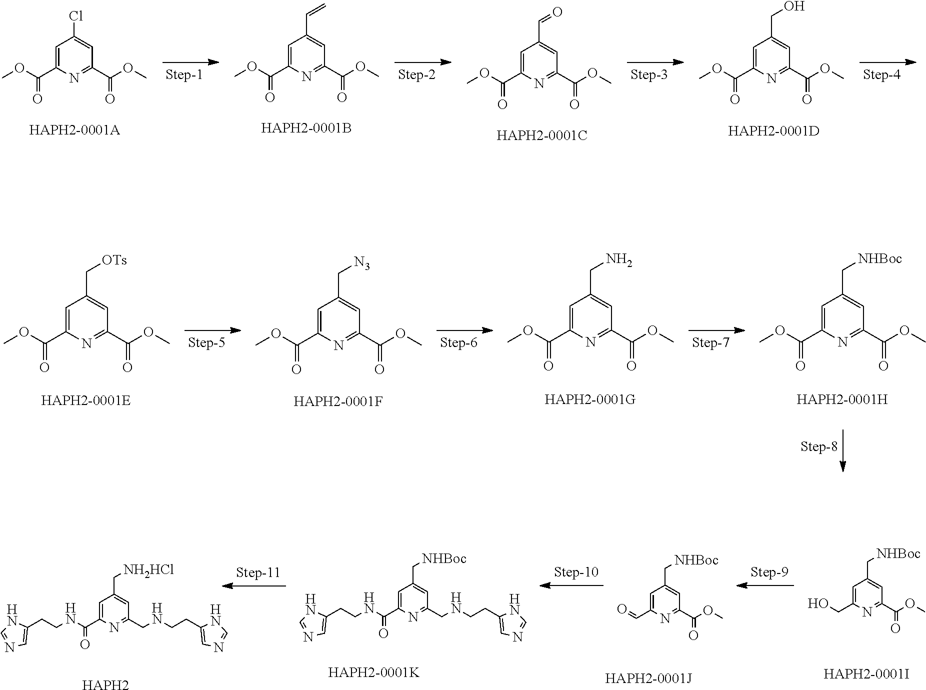

HAPH-2 hydrochloride was synthesized according to Scheme 3, through Steps 1-11 detailed below.

Step-1: To a stirred solution of Dimethyl-4-chloropyridine-2,6-dicarboxylate (1.0 g, 4.34 mmol) in 1,4-Dioxane (16.0 ml) and H2O (4 ml) was added K2CO3 (1.8 g, 13.04 mmol) followed by Potassium vinyltrifluoroborate (0.87 g, 6.52 mmol). Reaction mixture was degassed with N₂ for 15 min, then added Pd(dppf)Cl2. DCM (0.15 g, 0.217 mmol). It was heated to 85° C. for 10 hours. Completion of starting material was monitored by TLC. Then the reaction mixture was extracted with EtOAc (2×50 ml), washed with water (2×50 ml), followed by Brine (2×50 ml). The organic layer was dried over Na2SO4, filtered and evaporated under reduced pressure. Crude compound was purified by column chromatography and compound was eluted in 20% Ethyl acetate/Hexane to afford 0.3 g of Dimethyl 4-vinylpyridine-2,6-dicarboxylate as a White solid.

Step-2: To a stirred solution of Dimethyl 4-vinylpyridine-2,6-dicarboxylate (0.5 g, 2.26 mmol) in THF (10 ml) was added 4% OsO₄ in water (0.7 ml, 0.11 mmol), NMO (0.91 g, 6.78 mmol) at Room temperature. It was stirred for 4 hours at room temperature. Then it was extracted with EtOAc (2×50 ml), washed with water (2×50 ml) and Brine (2×10 ml). The Crude compound was dissolved in ACN (10 ml) and H₂O (5 ml), was added NaIO₄ (0.75 g, 3.39 mmol). It was stirred at RT for 4 h. Completion of starting material was monitored by TLC. Then the Reaction mixture was extracted with EtOAc (2×50 ml), washed with water (2×50 ml) and Brine (2×50 ml). The organic layer was dried over Na₂SO4, filtered and evaporated under reduced pressure. The Crude compound was purified by column and compound was eluted in 50% Ethyl acetate/Hexane to afford 0.3 g Dimethyl-4-formylpyridine-2,6-dicarboxylate as a white solid.

Step-3: To a stirred solution of Dimethyl-4-formylpyridine-2,6-dicarboxylate (0.3 g, 0.89 mmol) in THF (10 ml) was added NaBH₄ (0.16 g, 0.44 mmol) at 0° C. Reaction mixture was stirred at RT for one hour. TLC indicate completion of starting material. Reaction mixture was extracted with EtOAc (2×50 ml), washed with water (2×20 ml) and Brine (2×20 ml). The organic layer was dried over Na₂SO4, filtered and evaporated under reduced pressure. Crude compound was purified by column and compound was eluted in 80% Ethyl acetate/Hexane to afford 0.3 g of Dimethyl-4-formylpyridine-2,6-dicarboxylate a white solid.

Step-4: TEA (2.5 eq) and Tosyl chloride (1.1 eq) were added to a stirred solution of SM (1.8 g, 1 eq) in DCM at 0° C. RM was stirred at RT for 3 h. TLC showed completion of SM. Reaction mixture was extracted with DCM and washed water. Organic layer was dried over Na₂SO4 and concentrated. Crude compound was purified by column. Compound was eluted with 70% EA/Hexane.

Step-5: NaN3 (4 eq) was added to a stirred solution of SM (4.5 g, 1 eq) in DMF at RT. RM was stirred at 85° C. for 5 h. TLC showed completion of SM. Reaction mixture was extracted with EtOAC, washed water and Brine solution. Organic layer was dried over Na₂SO4 and concentrated. Crude compound was purified by column. Compound was eluted with 40% EA/Hexane.

Step-6: Pd/C (600 mg) was added to a stirred solution of SM (1.6 g, 1 eq) in MeOH at RT. RM was stirred at RT for 16 h under Hydrogen. TLC showed completion of SM. Reaction mixture was filtered through celite and washed with MeOH. Organic layer was concentrated to get 1 g of Amine compound. Crude was as such taken for next step.

Step-7: TEA (3 eq) and (Boc)₂O (1.2 eq) were added to a stirred solution of SM (1 g, 1 eq) in DCM at 0° C. RM was stirred at RT for 3 h. TLC showed completion of SM. Reaction mixture was extracted with DCM and washed water. Organic layer was dried over Na₂SO4 and concentrated. Crude compound was purified by column. Compound was eluted with 40% EA/Hexane.

Step-8: To a stirred solution of 4-(tert-Butoxycarbonylamino-methyl)-pyridine-2,6-dicarboxylic acid monomethyl ester (0.2 g) in DCM (4 ml) and MeOH (1 ml) was added NaBH₄ (21 mg) at 0° C. Reaction mixture was stirred at RT for 1 h. TLC indicate completion of starting material. Reaction mixture was extracted with DCM (2×10 ml), washed with water (2×10 ml). The organic layer was dried over Na₂SO4, filtered and evaporated under reduced pressure. Crude compound was purified by column and compound was eluted in 50% Ethyl acetate/Hexane to afford 0.15 g of 4-(tert-Butoxycarbonylamino-methyl)-6-hydroxymethyl-pyridine-2-carboxylic acid methyl ester as a white solid.

Step-9: To a stirred solution of 4-(tert-Butoxycarbonylamino-methyl)-6-hydroxymethyl-pyridine-2-carboxylic acid methyl ester (0.15 g) in 1,4-Dioxane (3.0 ml) was added SeO₂ (0.23 mmol) at RT. Reaction mixture was stirred at 80° C. for 2 h. TLC indicate completion of starting material. Reaction mixture was filtered through celite bed and was evaporated under reduced pressure. Crude compound was purified by column and compound was eluted in 10% Ethyl acetate/Pet ether to afford 0.125 g 4-(tert-Butoxycarbonylamino-methyl)-6-formyl-pyridine-2-carboxylic acid methyl ester as a white solid.

Step-10A: 4-(tert-Butoxycarbonylamino-methyl)-6-formyl-pyridine-2-carboxylic acid methyl ester (0.15 g) and histamine (0.57 mmol) was taken in HFIP (1.5 ml) and stirred for 6 hours at RT then added Sodium borohydride (0.35 mmol) and stirred for 16 hours. TLC indicates completion of starting material. Reaction mixture was evaporated under reduced pressure. Crude was purified through column Chromatography (Eluent: 25-30% MeOH in DCM) to afford 150 mg of 4-(tert-Butoxycarbonylamino-methyl)-6-{[2-(3H-imidazol-4-yl)-ethylamino]-methyl}-pyridine-2-carboxylic acid methyl ester.

Step-10B: To a stirred solution of 4-(tert-Butoxycarbonylamino-methyl)-6-{[2-(3H-imidazol-4-yl)-ethylamino]-methyl}-pyridine-2-carboxylic acid methyl ester (0.15 g) and histamine (1.5 mmol) was taken in MeOH (2.0 ml) stirred for 48 hours at RT. TLC indicates completion of starting material. Reaction mixture was evaporated under reduced pressure. Crude was purified through column Chromatography (Eluent: 30% MeOH in DCM) to afford 150 mg of {2-{[2-(3H-Imidazol-4-yl)-ethylamino]-methyl}-6-[2-(3H-imidazol-4-yl)-ethylcarbamoyl]-pyridin-4-ylmethyl}-carbamic acid tert-butyl ester. Crude was purified through Preparative HPLC.

Step-11: To a stirred solution of {2-{[2-(3H-Imidazol-4-yl)-ethylamino]-methyl}-6-[2-(3H-imidazol-4-yl)-ethylcarbamoyl]-pyridin-4-ylmethyl}-carbamic acid tert-butyl ester (0.15 g) was taken in (2.0 ml) and added Dioxane. HCl (0.5 ml) stirred for 4 hrs at RT. TLC indicated completion of starting material. Reaction mixture was evaporated under reduced pressure. The material was triturated with Diethyl ether (5.0 ml) filtered and dried under vacuum to achieve target compound.

HAPH-2 HCl was confirmed by ¹H NMR as shown in FIG. 4A-4C. Ammonium acetate was used to convert the hydrochloride salt to HAPH-2 base, and the conversion was confirmed by HPLC FIG. 4D.

### Example 6. Synthesis of HAPH-3

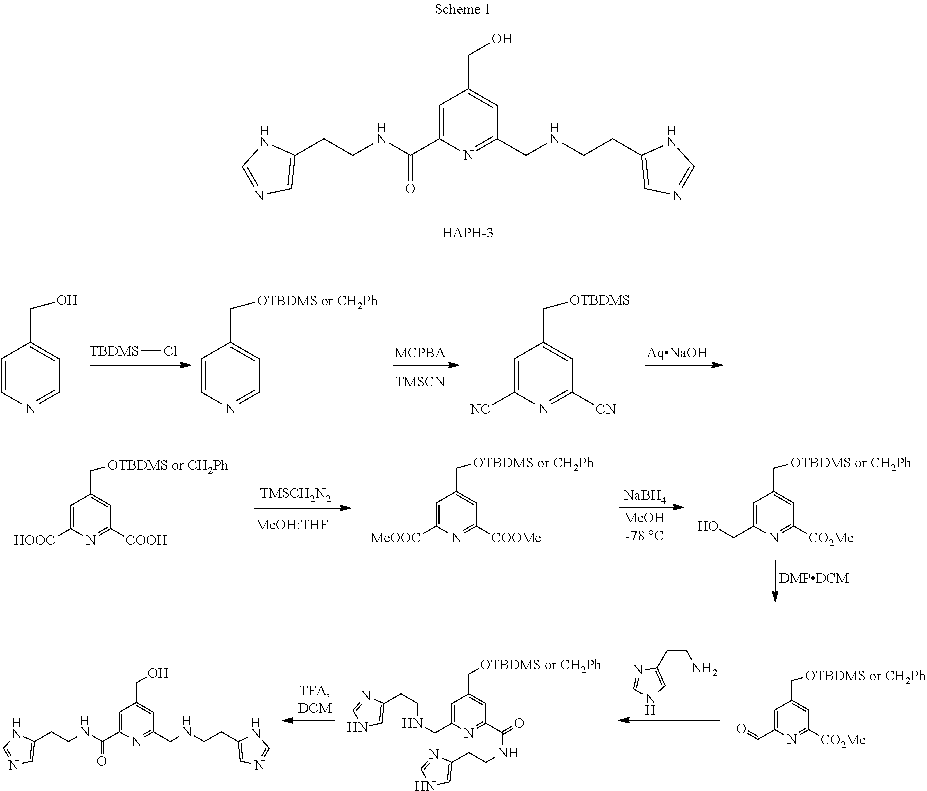

HAPH-3 may be synthesized by Scheme 1. Alternatively, HAPH-3 was synthesized according to Scheme 4 below with the detailed Step-1 through Step-8.

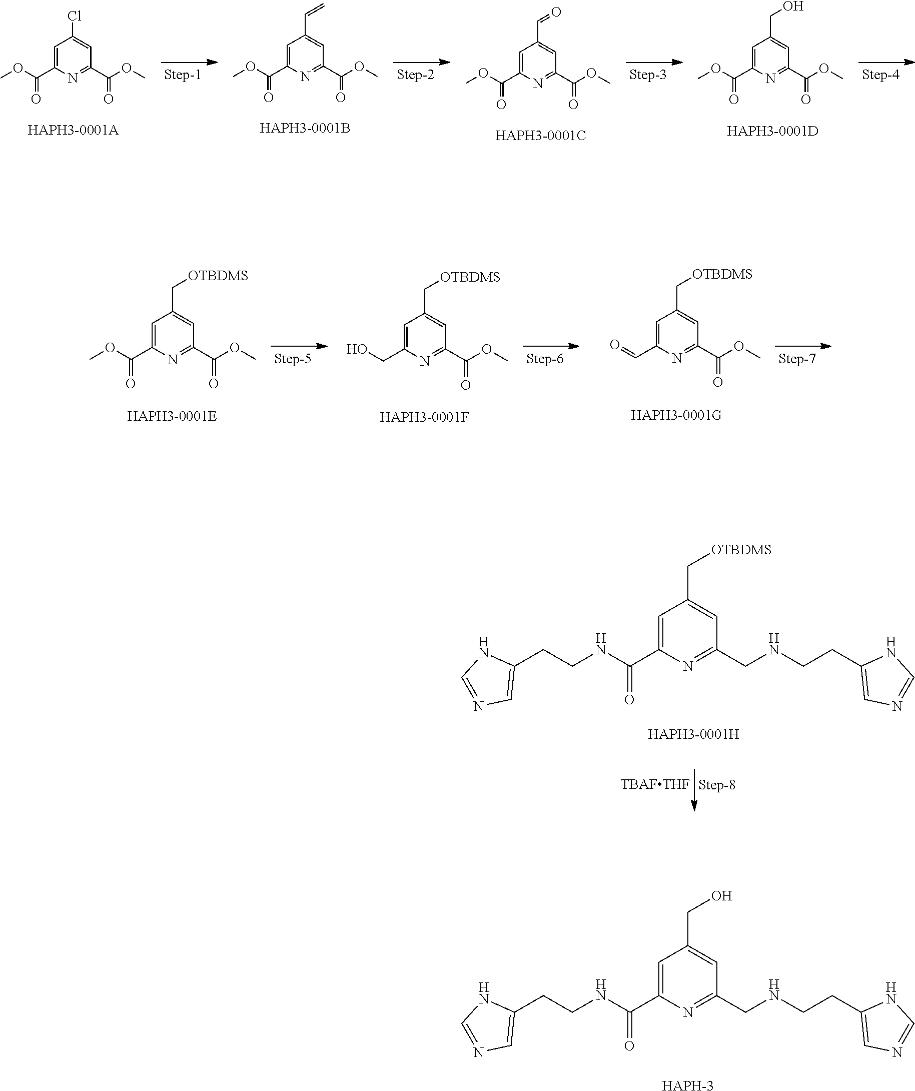

Step-1: To a stirred solution of Dimethyl-4-chloropyridine-2,6-dicarboxylate (1.0 g, 4.34 mmol) in 1,4-Dioxane (16.0 ml) and H₂O (4 ml) was added K₂CO₃ (1.8 g, 13.04 mmol) followed by Potassium vinyltrifluoroborate (0.87 g, 6.52 mmol). Reaction mixture was degassed with N2 for 15 min, then added Pd(dppf)Cl₂. DCM (0.15 g, 0.217 mmol). It was heated to 85° C. for 10 hours. Completion of starting material was monitored by TLC. Then the reaction mixture was extracted with EtOAc (2×50 ml), washed with water (2×50 ml), followed by Brine (2×50 ml). The organic layer was dried over Na₂SO₄, filtered and evaporated under reduced pressure. Crude compound was purified by column chromatography and compound was eluted in 20% Ethyl acetate/Hexane to afford 0.3 g of Dimethyl 4-vinylpyridine-2,6-dicarboxylate as a White solid.

Step-2: To a stirred solution of Dimethyl 4-vinylpyridine-2,6-dicarboxylate (0.5 g, 2.26 mmol) in THF (10 ml) was added 4%0.0 in water (0.7 ml, 0.11 mmol), NMO (0.91 g, 6.78 mmol) at Room temperature. It was stirred for 4 hours at room temperature. Then it was extracted with EtOAc (2×50 ml), washed with water (2×50 ml) and Brine (2×10 ml). The Crude compound was dissolved in ACN (10 ml) and H2O (5 ml), was added NaIO₄(0.75 g, 3.39 mmol). It was stirred at RT for 4 h. Completion of starting material was monitored by TLC. Then the Reaction mixture was extracted with EtOAc (2×50 ml), washed with water (2×50 ml) and Brine (2×50 ml). The organic layer was dried over Na₂SO₄, filtered and evaporated under reduced pressure. The Crude compound was purified by column and compound was eluted in 50% Ethyl acetate/Hexane to afford 0.3 g Dimethyl-4-formylpyridine-2,6-dicarboxylateas a White solid.

Step-3: To a stirred solution of Dimethyl-4-formylpyridine-2,6-dicarboxylate (0.3 g, 0.89 mmol) in THF (10 ml) was added NaBH₄ (0.16 g, 0.44 mmol) at 0° C. Reaction mixture was stirred at RT for one hour. TLC indicate completion of starting material. Reaction mixture was extracted with EtOAc (2×50 ml), washed with water (2×20 ml) and Brine (2×20 ml). The organic layer was dried over Na₂SO₄, filtered and evaporated under reduced pressure. Crude compound was purified by column and compound was eluted in 80% Ethyl acetate/Hexane to afford 0.3 g of Dimethyl-4-formylpyridine-2,6-dicarboxylate a white solid.

Step-4: To a stirred solution of Dimethyl-4-formylpyridine-2,6-dicarboxylate (0.3 g, 0.88 mmol) in DCM (8.0 ml) was added Imidazole (0.15 g, 2.21 mmol) at 0° C. and stirred for 15 minutes. Then TBDMS-Cl (0.16 g, 1.06 mmol) in DCM (2.0 mL) was added dropwise at 0° C. Reaction mixture was stirred at RT for 16 hours. TLC indicated completion of starting material. Reaction mixture was extracted with DCM (2×25 ml), washed with water (2×10 ml) and Brine (2×10 ml). The organic layer was dried over Na2SO4, filtered and evaporated under reduced pressure. Crude compound was purified by column and compound was eluted in 40% Ethyl acetate/Hexane to afford 0.3 g of Dimethyl 4-(((tert-butyldimethylsilyl)oxy)methyl)pyridine-2,6-dicarboxylate as White solid.

Step-5: To a stirred solution of Dimethyl-4-vinylpyridine-2,6-dicarboxylate (0.2 g, 0.56 mmol) in DCM (4 ml) and MeOH (1 ml) was added NaBH₄ (21 mg, 0.56 mmol) at 0° C. Reaction mixture was stirred at RT for 1 h. TLC indicated completion of starting material. Reaction mixture was extracted with DCM (2×10 ml), washed with water (2×10 ml). The organic layer was dried over Na₂SO₄, filtered and evaporated under reduced pressure. Crude compound was purified by column and compound was eluted in 50% Ethyl acetate/Hexane to afford 0.15 g of Methyl 4-(((tert-butyldimethylsilyl)oxy)methyl)-6-(hydroxymethyl) picolinate as a white solid.

Step-6: To a stirred solution of Methyl 4-(((tert-butyldimethylsilyl)oxy)methyl)-6-(hydroxymethyl) (0.15 g, 0.46 mmol) in 1,4-Dioxane (3.0 ml) was added SeO₂ (25 mg, 0.23 mmol) at RT. Reaction mixture was stirred at 80° C. for 2 h. TLC indicate completion of starting material. Reaction mixture was filtered through celite bed and was evaporated under reduced pressure. Crude compound was purified by column and compound was eluted in 10% Ethyl acetate/Pet ether to afford 0.125 g methyl 4-(((tert-butyldimethylsilyl)oxy)methyl)-6-formylpicolinate as a white solid.

Step-7A: methyl 4-(((tert-butyldimethylsilyl)oxy)methyl)-6-formylpicolinate (0.15 g, 0.48 mmol) and histamine (64 mg, 0.57 mmol) was taken in HFIP (1.5 ml) and stirred for 6 hours at RT then added Sodium borohydride (12 mg, 0.35 mmol) and stirred for 16 hours. TLC indicates completion of starting material. Reaction mixture was evaporated under reduced pressure. Crude was purified through column Chromatography (Eluent: 25-30% MeOH in DCM) to afford 150 mg of 4-(tert-Butyl-dimethyl-silanyloxymethyl)-6-{[2-(3H-imidazol-4-yl)-ethylamino]-methyl}-pyridine-2-carboxylic acid methyl ester.

Step-7B: To a stirred solution of 4-(tert-Butyl-dimethyl-silanyloxymethyl)-6-{[2-(3H-imidazol-4-yl)-ethylamino]-methyl}-pyridine-2-carboxylic acid methyl ester (0.15 g, 0.37 mmol) and histamine (171 mg, 1.5 mmol) was taken in MeOH (2.0 ml) stirred for 48 hours at RT. TLC indicated completion of starting material. Reaction mixture was evaporated under reduced pressure. Crude was purified through column Chromatography (Eluent: 30% MeOH in DCM) to afford 150 mg of 4-(tert-Butyl-dimethyl-silanyloxymethyl)-6-{[2-(3H-imidazol-4-yl)-ethylamino]-methyl}-pyridine-2-carboxylic acid [2-(3H-imidazol-4-yl)-ethyl]-amide.

Step-8: To a stirred solution of -(tert-Butyl-dimethyl-silanyloxymethyl)-6-{[2-(3H-imidazol-4-yl)-ethylamino]-methyl}-pyridine-2-carboxylic acid [2-(3H-imidazol-4-yl)-ethyl]-amide (0.15 g, 0.31 mmol) was taken in THF (2.0 ml) and added TBAF (0.5 ml) stirred for 4 hrs at RT. TLC indicates completion of starting material. Reaction mixture was evaporated under reduced pressure. Crude was purified through Preparative HPLC.

HAPH-3 was characterized by ¹H NMR, Mass Spectroscopy (MS), LCMS, and HPLC as shown in FIGS. 5A-5G. Thin layer chromatography (TLC) and PREP purification process were summarized as below.

TLC was performed with the following mobile phases: Step-1: Mobile phase 30% Ethyl acetate in Hexane with 0.4 Rf; Step-2: Mobile phase 50% Ethyl acetate in Hexane with 0.3 Rf; Step-3: Mobile phase 70% Ethyl acetate in Hexane with 0.3 Rf; Step-4: Mobile phase 30% Ethyl acetate in Hexane with 0.4 Rf; Step-5: Mobile phase 70% Ethyl acetate in Hexane with 0.3 Rf; Step-6: Mobile phase 2% MeOH in MDC with 0.5 Rf; Step-7A: Mobile phase 5% MeOH in MDC with 0.2 Rf; Step-7B: Mobile phase 10% MeOH in MDC with 0.2 Rf, two times elution; and Step-8: Mobile phase 20% MeOH in MDC with 0.1 Rf, two times elution. PREP Conditions included trials mini preconditions A-F. Mini Prep conditions A With Gradient Program: Time/Pump B: 0/5,4/5, 16/25, 17/100,22/100,22.5/5,28/5; Flow Rate: 15 ml/Min; Column: Xbridge Bridge C18 (*250*10 um); Buffers Used: Pump A: 0.1% TFA In Water And Pump B: 100% Acetonitrile. Mini Prep Conditions B With Gradient Program: Time/Pump B: 0/5,4/5, 16/25, 17/100,22/100,22.5/5,28/5; Flow Rate: 15 ml/Min; Column: Xbridge Bridge C18 (*250*10 um); Buffers Used Included Pump A: 10 mm Ammonium Acetate in Water And Pump B: 100% Acetonitrile. Mini Prep Conditions C Had Gradient Program: Time/Pump B: 0/5,4/5, 16/25, 17/100,22/100,22.5/5,28/5; Flow Rate: 15 ml/Min; Column: Xbridge Bridge C18 (*250*10 μm); Buffers used included Pump A: 10 mm Ammonium Formate In Water And Pump B: 100% Acetonitrile. Mini Prep Conditions D Had Gradient Program; Time/Pump B: 0/5,4/5, 16/25, 17/100,22/100,22.5/5,28/5; Flow Rate: 15 ml/Min; Column: Xbridge Bridge C18 (*250*10 um); Buffers Used Included Pump A: 0.1% TFA in Water and Pump B: Acetonitrile+MeOH. Mini Prep Conditions E Had Gradient Program: Time/Pump B: 0/5,4/5, 16/25, 17/100,22/100,22.5/5,28/5; Flow Rate: 15 ml/Min; Column: X-Select C18 (*250*10 um); Buffers Used Included Pump A: 0.1% TFA in Water and Pump B: Acetonitrile+MeOH. Mini Prep Conditions F Included A Gradient Program Of Time/Pump B: 0/5,4/5, 16/25, 17/100,22/100,22.5/5,28/5; Flow Rate: 15 ml/Min; Column: X-Select C18 (*250*10 um); Buffers Used Included Pump A: 0.1% Tfa In Water And Pump B: Acetonitrile. Mini Prep Conditions G Had A Gradient Program Of Time/Pump B: 0/5,4/5, 16/25, 17/100,22/100,22.5/5,28/5; Flow Rate: 15 ml/Min; Column: X-Select C18 (*250*10 μm); Buffers Used Included Pump A: 10 mm Abc In Water And Pump B: Acetonitrile. The finalized PREP condition was summarized in TABLE 3.

TABLE 3
Purification Details of HAPH-3
Purification details

| | | Gradient | | |
|---|---|---|---|---|
| Compound | | table | | |
| ID | HAPH-III | TIME(min) | % A | % B |
| column ID | XBRIDGE C18 (250*21 | 0 | 95 | 5 |
| | m*10 u) | | | |
| Flow rate | 15 ml/min | 2 | 95 | 5 |
| Mobile phase | | 17 | 90 | 10 |
| Phase A | 10 mm ABC in water | 19 | 90 | 10 |
| Phase B | HPLC grade Acetonitrile | 21 | 5 | 90 |
| Gradient: | Method in the table | 25 | 5 | 95 |
| | | 26 | 95 | 5 |
| | | 30 | 95 | 5 |

Retention time - 30 Min

### Example 7. Synthesis of HAPH-4 and HAPH-5

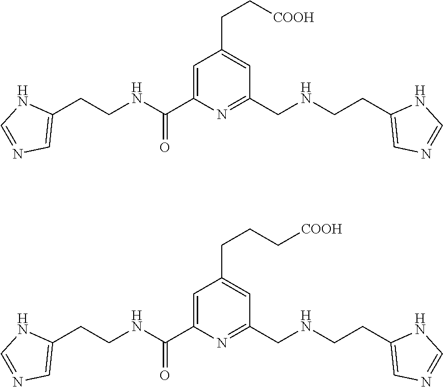 [SMILES: O=C(O)CCc1cc(CNCCc2cnc[nH]2)nc(C(=O)NCCc2cnc[nH]2)c1 ; O=C(O)CCCc1cc(CNCCc2cnc[nH]2)nc(C(=O)NCCc2cnc[nH]2)c1]

HAPH-4 and HAPH-5 are synthesized according to FIG. 2A.

### Example 8. Synthesis of HAPH-6 and HAPH-7

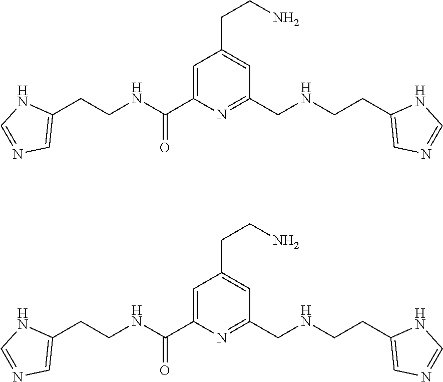 [SMILES: NCCc1cc(CNCCc2cnc[nH]2)nc(C(=O)NCCc2cnc[nH]2)c1 ; NCCc1cc(CNCCc2cnc[nH]2)nc(C(=O)NCCc2cnc[nH]2)c1]

HAPH-6 and HAPH-7 are synthesized according to FIG. 2B.

### Example 9. Synthesis of HAPH-8 and HAPH-9

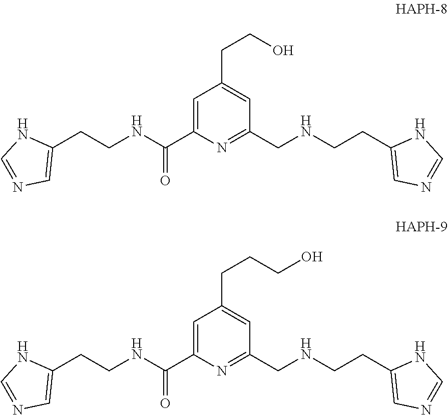 [SMILES: O=C(NCCc1cnc[nH]1)c1cc(CCO)cc(CNCCc2cnc[nH]2)n1 ; O=C(NCCc1cnc[nH]1)c1cc(CCCO)cc(CNCCc2cnc[nH]2)n1]

HAPH-8 and HAPH-9 are synthesized according to FIG. 2C.

### Example 10. Synthesis of Novel Intermediates HAPH-10, HAPH-11 and HAPH-12

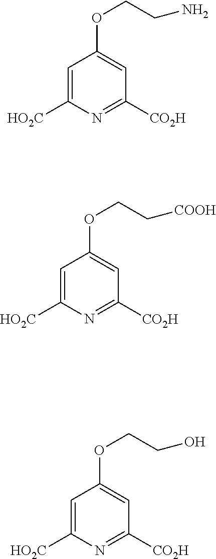 [SMILES: NCCOc1cc(C(=O)O)nc(C(=O)O)c1 ; O=C(O)CCOc1cc(C(=O)O)nc(C(=O)O)c1 ; O=C(O)c1cc(OCCO)cc(C(=O)O)n1]

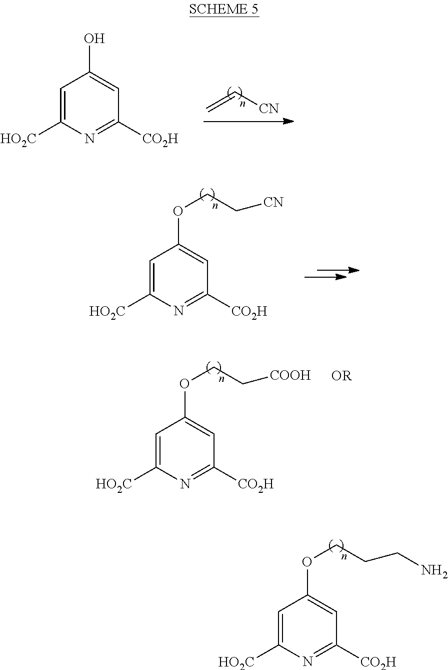

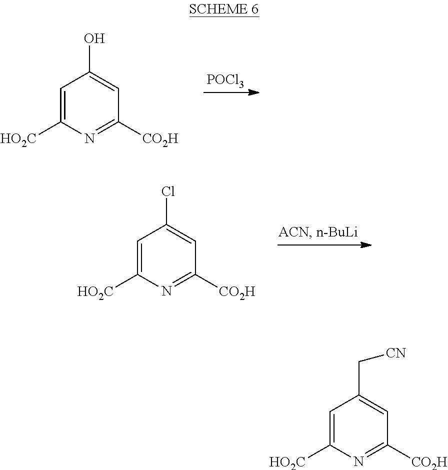

Novel intermediates HAPH-10, HAPH-11, and HAPH-12 are synthesized by using intermediates synthesized through Schemes 5-6.

### Example 11. Synthesis of HAPH-2 Azide

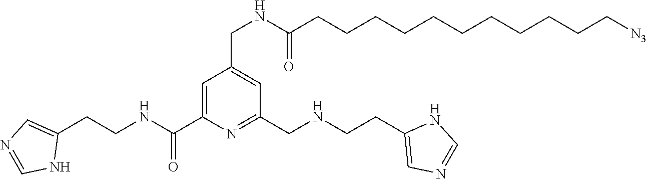 [SMILES: [N-]=[N+]=NCCCCCCCCCCCC(=O)NCc1cc(CNCCc2cnc[nH]2)nc(C(=O)NCCc2cnc[nH]2)c1]

### HAPH-2 Azide

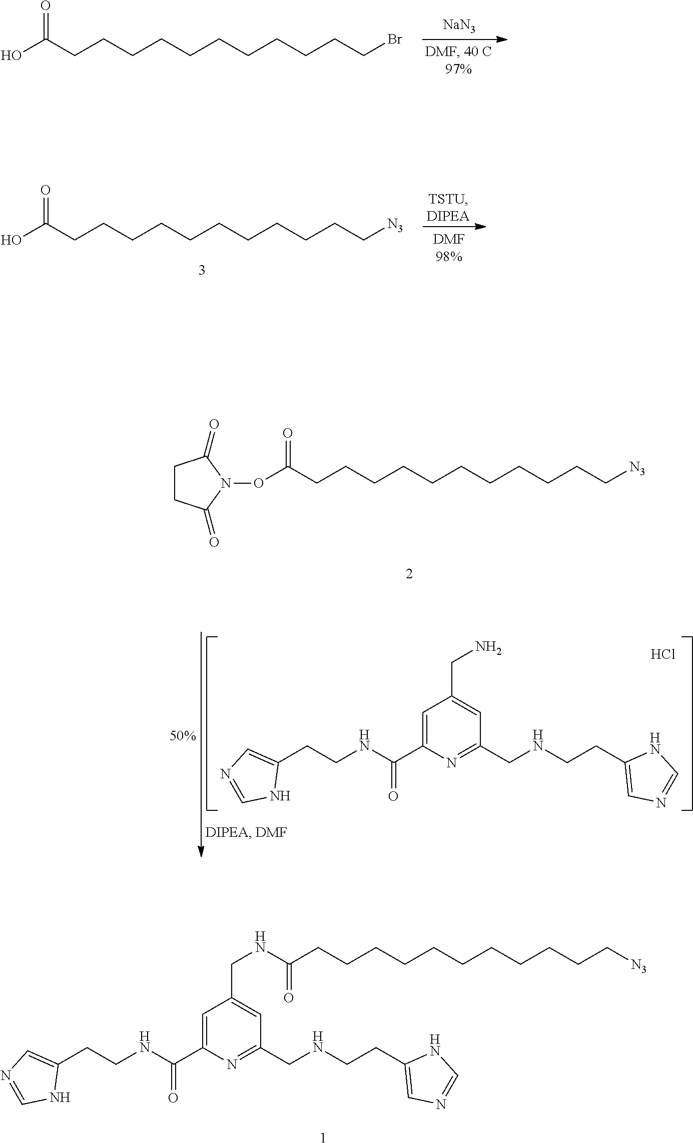

HAPH-2 Azide was synthesized according to Scheme 7 and detailed as following. All anhydrous solvents used in the following reactions were obtained from commercial sources or freshly distilled over calcium hydride. ¹H and ³¹P NMR spectra were recorded on a Bruker 500 or Varian 600 MHz spectrometer. 1H NMR chemical shifts are relative to CDCl₃ (δ=7.26 ppm), CD₃OD (δ=3.31 ppm) or D₂O (δ=4.79 ppm). High-resolution mass spectrometry (HRMS) spectra were obtained on an Applied Biosystems 4800 MALDI-TOF/TOF mass spectrometer (Applied Biosystems, Foster City, CA).

### 12-azidododecanoic acid (3)

### 2,5-dioxopyrrolidin-1-yl 12-azidododecanoate (2)

To a stirring solution of 12-bromodecanoic acid (1.5 g, 5.37 mmol) in anhydrous DMF (20 mL), was added NaN₃ under Argon at 40° C. The reaction was stirred for 12 h, then, the reaction solution was diluted with EtOAc (30 mL), washed sequentially with 1N HCl (aq) (20 mL, 3×), ddH₂O (aq) (20 mL, 2×) and Brine (20 mL, 1×). The organic layer was dried over Na₂SO₄, EtOAc was removed under reduced pressure and the isolated product was dried to give a waxy white solid in a 96.8% yield.

TLC: Rf=0.33 (50% EtOAc in Hexanes, visualization by reducing in 10% Ph₃P in CH₂Cl₂ then stained using ninhydrin)

### ¹H NMR (500 MHz, Chloroform-d) δ 3.25 (t, J=7.0 Hz, 2H), 2.35 (t, J=7.5 Hz, 2H), 1.61 (dp, J=21.8, 7.1 Hz, 5H), 1.29 (d, J=15.4 Hz, 17H).

Compound 3 (1.296 g, 5.37 mmol), TSTU (1.779 g, 5.91 mmol) and DIPEA (1.389 g, 10.7 mmol) were combined in anhydrous DMF (16 mL) with molecular sieves under Argon and the reaction was stirred at room temperature for 12 hours. The reaction was filtered through silica and activated charcoal and the resulting filtrate was diluted with EtOAc (50 mL) and washed sequentially with 1N HCl (aq) (30 mL, 3×), sat. NaHCO3(aq) (30 mL, 2×) and Brine (30 mL, 1×). The organic layer was dried over Na₂SO₄, EtOAc was removed under reduced pressure and resulted in an orange solid in 98% yield.

TLC: Rf=0.74 (50% EtOAc in Hexanes, visualization by reducing in 10% Ph₃P in CH₂Cl₂ then stained using ninhydrin or PMA)

### ¹H NMR (500 MHz, Chloroform-d) δ 3.25 (t, J=7.0 Hz, 2H), 2.88-2.78 (m, 4H), 2.60 (t, J=7.5 Hz, 2H), 1.74 (p, J=7.5 Hz, 2H), 1.63-1.53 (m, 6H), 1.44-1.23 (m, 16H).

### N-(2-(1H-imidazol-5-yl)ethyl)-6-(((2-(1H-imidazol-5-yl)ethyl)amino)methyl)-4-((12-azidododecanamido)methyl)picolinamide (HAPH-2 Azide)

To a stirring solution of HAPH-2 (83.4 mg, 0.152 mmol) and DIPEA (0.317 mL, 0.303 mmol) in anhydrous DMF (1.5 mL) a dry solution of Compound 2 (51.2 mg, 0.152 mmol) in anhydrous DMF (1.7 mL) under Argon at room temperature for 18 hours. The reaction solution was diluted with EtOAc (20 mL), and washed sequentially with 1N HCl (aq) (10 mL, 3×), and Brine (10 mL, 1×). The organic layer was dried over Na₂SO₄, EtOAc was removed under reduced pressure and the resulting oil was chromatographed via silica-gel column (5% MeOH in CH₂Cl₂→70% MeOH in CH₂Cl₂+0.1% TFA). The isolated product was dried to give a yellow oil in 49.7% yield.

TLC: Rf=0.13 (70% MeOH/CH₂Cl₂+0.1% NH₄OAc, visualization by reducing in 10% Ph₃P in CH₂Cl₂ then stained using ninhydrin or PMA).
¹H NMR 500 MHz, CD3OD) δ 7.94 (s, 1H), 7.64 (d, J=10.7 Hz, 2H), 7.44 (s, 1H), 6.92 (d, J=4.8 Hz, 2H), 4.47 (s, 2H), 4.16 (s, 2H), 3.70 (t, J=7.2 Hz, 2H), 3.28 (t, J=6.8 Hz, 2H), 3.11 (t, J=7.5 Hz, 2H), 2.96 (d, J=8.2 Hz, 4H), 2.31 (t, J=7.6 Hz, 2H), 1.73-1.53 (m, 4H), 1.34 (d, J=12.4 Hz, 15H).
HRMS (MALDI): m/z calculated for C₃₀H₄₆N₁₁O₂⁺ [M+H]: 592.3836, found 592.38586. Full characterizations of HAPH-2 Azide were provided in FIGS. 6A-6E.

TABLE 4
HPLC METHODS
Column: Phenomenex Luna 5 μm C18(2) 100 Å
Dimensions: 150 × 4.6 mm

| Time | % 1% TFA in ddH2O | % Acetonitrile | Flow Rate (mL/min) |
|---|---|---|---|
| 0 | 95 | 5 | 1 |
| 5 | 95 | 5 | 1 |
| 15 | 5 | 95 | 1 |
| 20 | 5 | 95 | 1 |
| 21 | 95 | 5 | 1 |
| 24 | 95 | 5 | 1 |

### Example 12. Scaled Up Synthesis

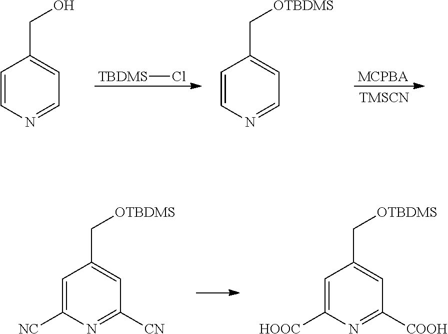

Various critical intermediates are tested for scaled up. For example, the intermediate for synthesis of HAPH-3 are synthesized in large or industrial scale.

### Example 13. Synthesis of HAPH-2 Ethyne

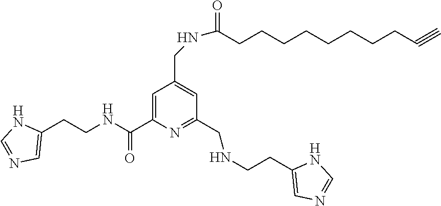 [SMILES: C#CCCCCCCCCC(=O)NCc1cc(CNCCc2cnc[nH]2)nc(C(=O)NCCc2cnc[nH]2)c1]

HAPH-2 Ethyne is synthesized through Scheme 9, wherein HAPH-2 is reacted with 2,5-dioxopyrrolidin-1-yl undec-10-ynoate and N,N-diisopropylethylamine (DIPEA) in DMF under appropriate conditions. The yield is over 50%.

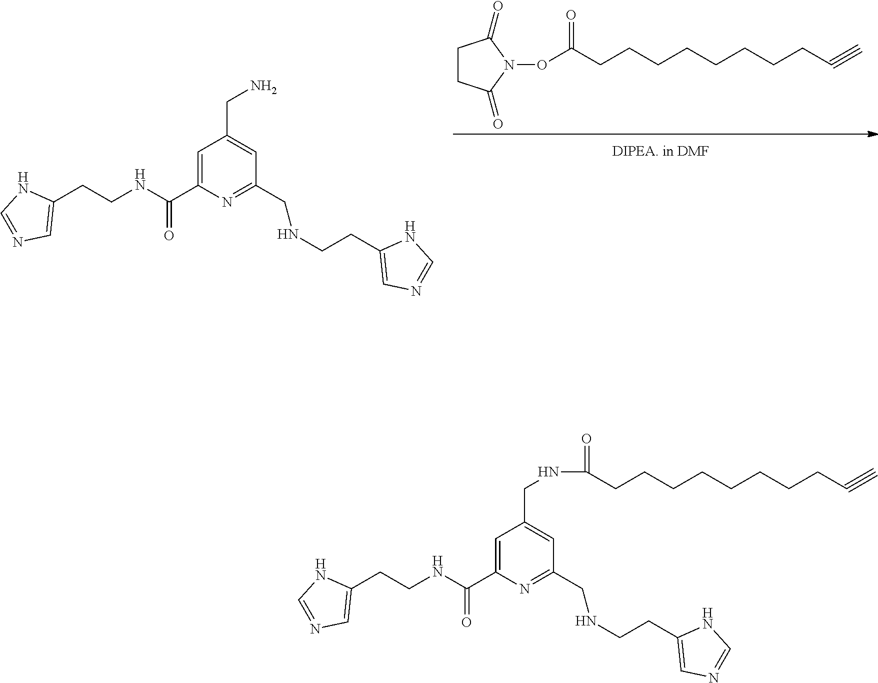 [SMILES: NCc1cc(CNCCc2cnc[nH]2)nc(C(=O)NCCc2cnc[nH]2)c1 ; C#CCCCCCCCCC(=O)ON1C(=O)CCC1=O ; C#CCCCCCCCCC(=O)NCc1cc(CNCCc2cnc[nH]2)nc(C(=O)NCCc2cnc[nH]2)c1]

### Example 14. Synthesis of Metal Chelates

Novel Metal chelates of HAPH analogs are synthesized by chelating with Cr, Mn, Co, Ni, Zn and Ag, specifically with Cr (III), Cr (VI), Mn (II), Mn (III), Mn (VI), Co (II), Co (III), Ni (II), Sc (II), Zn (II), Ga (III), In (III), Y (III), and Ag (I) through suitable chemical reactions. For example, synthesis of Cu^{II} HAPH was recited here for your reference: solution comprising Cu(NO₃)₂ (0.885M) was pipetted into HAPH solution (18 mg dissolved in 10 ml of water), such that 20% excess free ligand was present. In an alternate approach an excess of standard Cu^{II} was added to HAPH solution to form 1:1 complex. Excess Cu^{II} was precipitated by raising the pH to above 10. The insoluble Cu(OH)₂ was then removed by filtration with Milipore filters (0.47 μm mesh) and the pH of the solution was adjusted to desired range.)

Example 15. Preparation of Compositions and Dosage forms of HAPH Analogs and/or Metal Chelates Thereof

HAPH analogs and/or metal chelates are used as pharmaceutical active, as diagnostic active, and/or as theranostic active. These actives are formulated for administration through either parenteral or enteral routes. For a parenteral formulation, the active is combined with a suitable solvent at a pharmaceutically relevant concentration and formulated with additional suitable excipients and/or carriers. For intravenous administration, the composition can be further diluted with an infusion fluid, such as normal saline, before infusion into a patient. Alternatively, the composition can be formulated for subcutaneous injection administration. The patient receives an adequate dosage at an optimized frequency, necessary to show physiologically relevant suppression of tumor growth and/or spread. Alternatively, the formulation can be given in a ready to use unit dosage form.

Example 16. Therapeutic Applications of HAPH Analogs and/or Metal Chelates Thereof

Study is conducted to evaluate the pharmacodynamics of HAPH analogs, and their metal chelates. Assays evaluating cell proliferation, cell size, and cell senescence are performed by treating established cancer cell lines e.g. BSMZ, a HER2⁺ breast cancer cell line, with increasing doses of a testing HAPH analog, or a metal chelate thereof. Similarly, LNCaP, a PSMA⁺ prostate cancer cell line is treated with the testing HAPH analog, or its metal chelate. At pre-specified end points of treatment, assessment is done to measure treatment efficacies. Further, treatment efficacy and tolerability assessments are examined in animal models. In PDX mouse models, the testing active is administered at varying dosages. The serum concentrations of the analogs or chelates are measured using immunoassay. Assessments of tumor volume and size is determined to evaluate the efficacy of the treatments. The results from these experiments can be used to correlate drug sensitivity and drug dosage for clinical applications.

Example 17. Diagnostic and/or Theranostic Applications of HAPH Metal Chelates

HAPH analogs chelated with metal ion radioisotopes are used for diagnostic and/or theranostic imaging. HAPH analogs chelated with metal radioisotopes, such as ⁶⁴Cu, ⁶⁷Cu, ⁴³Sc, ⁴⁴Sc, ⁴⁶Sc, ⁴⁷Sc, or ⁴⁸Sc, are tested for use in theranostic positron emission tomography (PET). Tumor cell lines or animal models treated or injected with radioisotope derivatized HAPH analogs or chelates are tested initially to assess the efficacy of the treatment and specificity of imaging using PET scans. Further, computed tomography can be combined with PET scans to provide more assessments on tumor growth and/or spread.

Alternatively, HAPH analogs or metal chelates can be labelled with fluorescent probe to assess cellular uptake of HAPH, and to image and provide diagnosis of tumor growth and/or spread.

1. A compound of Formula (I), a pharmaceutically acceptable salt, a polymorph, a stereoisomer, a solvate or a prodrug thereof, 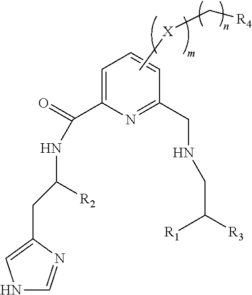
wherein
R₁ and R₃ each is independently selected from the substituted or unsubstituted group consisting of hydrogen, halogen, —NH₂, —C(O)NH₂, —C(O)OH, —C(O)OMe, —C(O)R₅, —C(O)NR₅, —OC(O)R₅, —C(O)OR₅, —(C₁-C₆ alkylene), —C₁-C₆ alkyl, —C₂-C₆ alkenyl, —C₂-C₆ alkynyl, —C₁-C₆ alkoxy, —C₁-C₆ heteroalkyl, —C₃-C₁₂ cycloalkyl, —C₆-C₁₄ aryl, —C₆-C₁₄ aryloxy, —C₆-C₁₄ aryl, 3-12 membered heterocyclyl, 3-12 membered heterocycloalkyl, 5-14 membered heteroaryl, —SH, —SMe, —SR₅, —S(O)₂R₅, —SR₅-aryl, and SR₅-aryl-O—R₅; and
R₂ is selected from the substituted or unsubstituted group consisting of hydrogen, halogen, —C₁-C₆ alkyl, —C₂-C₆ alkenyl, and —C₂-C₆ alkynyl;
The motif Xm-(CH₂)n-R₄ comprises para- or meta-position relative to N on the pyridine ring;
X is —O—, —CH₂NR₅—, CH₂O—, —CH₂C(O)—, CH₂—NR₅—C(O)—, —NR₅—, —CH₂S—, or —S—;
m is the number 0 or 1, and when m is 0, the methylene group is directly connected to the pyridine ring;
n is zero or an integer of 1, 2, 3, 4, 5, 6, 7, 8, 9, or 10;
R₄ is selected from the substituted or unsubstituted group consisting of hydrogen, hydroxyl, —NH₂, —C(O)NH₂, —NHC(O), —C(O)OH, —C(O)OMe, —C(O)R₅, —C(O)NR₅, —OC(O)R₅, —C(O)OR₅—, —N₃, —(CH₂)₁₀—N₃; —NH—C(O)—(CH₂)₁₁—N₃, —C₁-C₁₅ alkylazide, —C≡CH, (CH₂)₁₀—C≡CH; —NH—C(O)—(CH₂)₁₁—C≡CH; —C₁-C₁₅—C≡CH; —SH, —SMe, —SR₅, —S(O)₂R₅, —SR₅-aryl, SR₅-aryl-O—R₅, —C₁-C₁₅ alkyl, —C₂-C₁₅ alkenyl, —C₂-C₁₅ alkynyl, and phosphate; and
wherein each R₅ is selected independently from the group consisting of hydrogen, C₁-C₆ alkyl, C₂-C₆ alkenyl, and C₂-C₆ alkynyl;
with the proviso that when m=n=0, R₁ is not selected from the group consisting of —NH₂, —SH, —SCH₃, SCH₂C₆H₄OCH₃, and 5-imidazolyl.

2. (canceled)

3. The compound of claim 1, wherein the compound has a structure of Formula (III), 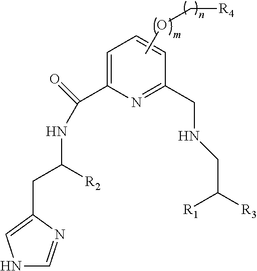
wherein
X is —O—;
m is the number 0 or 1, and when m is 0, the methylene group is directly connected to the pyridine ring;
n is zero or an integer of 1, 2, 3, 4, 5, 6, 7, 8, 9, 10;
R₄ is selected from the substituted or unsubstituted group consisting of hydrogen, hydroxyl, —NH₂, —C(O)NH₂, —NHC(O), —C(O)OH, —C(O)OMe, —C(O)R₅, —C(O)NR₅, —OC(O)R₅, —C(O)OR₅—, —N₃, —(CH₂)₁₀—N₃; —NH—C(O)—(CH₂)₁₁—N₃, —C₁-C₁₅ alkylazide, —C≡CH, —(CH₂)₁₀—C≡CH; —NH—C(O)—(CH₂)₁₁—C≡CH; —C₁-C₁₅—C≡CH; —SH, —SMe, —SR₅, —S(O)₂R₅, —SR₅-aryl, SR₅-aryl-O—R₅, —C₁-C₁₅ alkyl, —C₂-C₁₅ alkenyl, —C₂-C₁₅ alkynyl, and phosphate;
with the proviso that m and n are not zero at the same time.

4. The compound of claim 1, wherein the compound has a structure of Formula (IV).

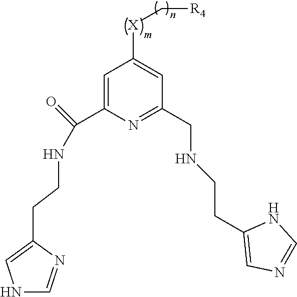

5. The compound according to Formula (I), having one of the following structures:

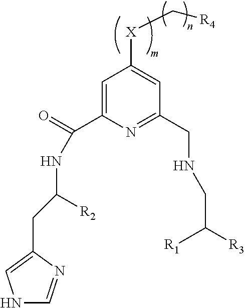

| NAME | R₁ | R₂ | R₃ | R₄ | X | m | n |
|---|---|---|---|---|---|---|---|
| HAPH-2 | 5-imidazolyl | H | H | NH₂ | N/A | 0 | 1 |
| HAPH-2 | 5-imidazolyl | H | H | NH₂ HCl | N/A | 0 | 1 |
| HCl Salt | | | | | | | |
| HAPH-2 | 5-imidazolyl | H | H | Na | CH₂—NHC(O) | 0 | 11 |
| Azide | | | | | | | |
| HAPH-2 | 5-imidazolyl | H | H | CΞC | CH₂—NHC(O) | 0 | 11 |
| Ethyne | | | | | | | |
| HAPH-2 | 5-imidazolyl | H | H | N₃ | N/A | 0 | 1 |
| Methylene | | | | | | | |
| Azide | | | | | | | |
| HAPH-3 | 5-imidazolyl | H | H | OH | N/A | 0 | 1 |
| HAPH-4 | 5-imidazolyl | H | H | COOH | N/A | 0 | 2 |
| HAPH-5 | 5-imidazolyl | H | H | COOH | N/A | 0 | 3 |
| HAPH-6 | 5-imidazolyl | H | H | NH₂ | N/A | 0 | 2 |
| HAPH-7 | 5-imidazolyl | H | H | NH₂ | N/A | 0 | 3 |
| HAPH-8 | 5-imidazolyl | H | H | OH | N/A | 0 | 2 |
| HAPH-9 | 5-imidazolyl | H | H | OH | N/A | 0 | 3 |
| HAPH-10 | 5-imidazolyl | H | H | NH₂ | O | 1 | 2 |
| HAPH-11 | 5-imidazolyl | H | H | COOH | O | 1 | 2 |
| HAPH-12 | 5-imidazolyl | H | H | OH | O | 1 | 2 |
| HAPH-13 | 5-imidazolyl | H | H | COOH | N/A | 0 | 1 |
| AMPHIS-1N | —NH₂ | —C(O)OCH₃ | H | NH₂ | N/A | 0 | 1 |
| AMPHIS-1O | —NH₂ | —C(O)OCH₃ | H | OH | N/A | 0 | 1 |
| AMPHIS-1A | —NH₂ | —C(O)OCH₃ | H | COOH | N/A | 0 | 1 |
| PYML-1N | NH₂ | —C(O)OH | —C(O)NH₂ | NH₂ | N/A | 0 | 1 |
| PYML-1O | NH₂ | —C(O)OH | —C(O)NH₂ | OH | N/A | 0 | 1 |
| PYML-1A | NH₂ | —C(O)OH | —C(O)NH₂ | COOH | N/A | 0 | 1 |
| SAPH-1N | —SMe | H | H | NH₂ | N/A | 0 | 1 |
| SAPH-1O | —SMe | H | H | OH | N/A | 0 | 1 |
| SAPH-1A | —SMe | H | H | COOH | N/A | 0 | 1 |
| SAPH-2N | —SCH₂C₆H₄OMe | H | H | NH₂ | N/A | 0 | 1 |
| SAPH-2O | —SCH₂C₆H₄OMe | H | H | OH | N/A | 0 | 1 |
| SAPH-2A | —SCH₂C₆H₄OMe | H | H | COOH | N/A | 0 | 1 |
| SAPH-3N | —SH | H | H | NH₂ | N/A | 0 | 1 |
| SAPH-3O | —SH | H | H | OH | N/A | 0 | 1 |
| SAPH-3A | —SH | H | H | COOH | N/A | 0 | 1 |

6. A metal chelate of Formula (V), wherein the metal M is selected from the group consisting of Fe, Cu, Sc, Cr, Mg, Mn, Co, Ni, Zn, Ga, In, Y and Ag; 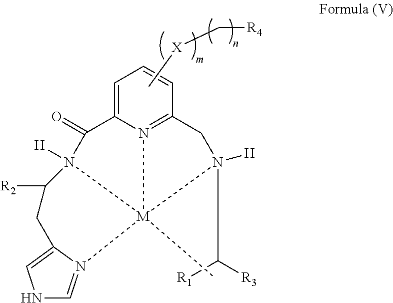
and wherein
R₁ and R₃ each is independently selected from the substituted or unsubstituted group consisting of hydrogen, halogen, —NH₂, —C(O)NH₂, —C(O)OH, —C(O)OMe, —C(O)R₅, —C(O)NR₅, —OC(O)R₅, —C(O)OR₅, —(C₁-C₆ alkylene), —C₁-C₆ alkyl, —C₂-C₆ alkenyl, —C₂-C₆ alkynyl, —C₁-C₆ alkoxy, —C₁-C₆ heteroalkyl, —C₃-C₁₂ cycloalkyl, —C₆-C₁₄ aryl, —C₆-C₁₄ aryloxy, —C₆-C₁₄ aryl, 3-12 membered heterocyclyl, 3-12 membered heterocycloalkyl, 5-14 membered heteroaryl, —SH, —SMe, —SR₅, —S(O)₂R₅, —SR₅-aryl, and SR₅-aryl-O—R₅; and
R₂ is selected from the substituted or unsubstituted group consisting of hydrogen, halogen, —C₁-C₆ alkyl, —C₂-C₆ alkenyl, and —C₂-C₆ alkynyl;
X is —O—, —CH₂NR₅—, CH₂O—, —CH₂C(O)—, CH₂—NR₅—C(O)—, —NR₅—, —CH₂S—, or —S—;
m is the number 0 or 1, and when m is 0, the methylene group is directly connected to the pyridine ring;
n is zero or an integer of 1, 2, 3, 4, 5, 6, 7, 8, 9, or 10;
R₄ is selected from the substituted or unsubstituted group consisting of hydrogen, hydroxyl, —NH₂, —C(O)NH₂, —NHC(O), —C(O)OH, —C(O)OMe, —C(O)R₅, —C(O)NR₅, —OC(O)R₅, —C(O)OR₅—, —N₃, —(CH₂)₁₀—N₃; —NH—C(O)—(CH₂)₁₁—N₃, —C₁-C₁₅ alkylazide, —C≡CH, —(CH₂)₁₀—C≡CH; —NH—C(O)—(CH₂)₁₁—C≡CH; —C₁-C₁₅—C≡CH; —SH, —SMe, —SR₅, —S(O)₂R₅, —SR₅-aryl, SR₅-aryl-O—R₅, —C₁-C₁₅ alkyl, —C₂-C₁₅ alkenyl, —C₂-C₁₅ alkynyl, and phosphate; and
wherein each R₅ is selected independently from the group consisting of C₁-C₆ alkyl, C₂-C₆ alkenyl, and C₂-C₆ alkynyl;
with the proviso that when metal is Fe or Cu and m=n=0, R₁ is not —SH, SMe, —SCH₂C₆H₄OMe or 5-imidazolyl.

7. The metal chelate of claim 6, wherein the metal is a metal ion comprises Fe (II), Fe (III), Cu (II), Cr (III), Cr (VI), Mg (II), Mn (II), Mn (III), Mn (VI), Co (II), Co (III), Sc (II), Ni (II), Zn (II), Ga (III), In (III), Y (III) and Ag (I).

8. The metal chelate of claim 6, wherein the metal is an isotope or a radioisotope form.

9. (canceled)

10. The metal chelate of claim 6, wherein the metal chelate is of Formula (VI):

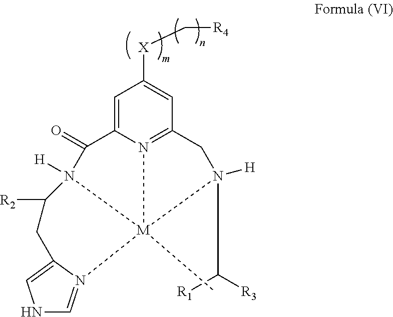

| NAME | R₁ | R₂ | R₃ | R₄ | X | m | n |
|---|---|---|---|---|---|---|---|
| AMPHIS | —NH₂ | —C(O)OCH₃ | H | H | N/A | 0 | 0 |
| PYML | NH₂ | —C(O)OH | —C(O)NH₂ | H | N/A | 0 | 0 |
| SAPH-1 | —SMe | H | H | H | N/A | 0 | 0 |
| SAPH-2 | —SCH₂C₆H₄OMe | H | H | H | N/A | 0 | 0 |
| SAPH-3 | —SH | H | H | H | N/A | 0 | 0 |
| HAPH-1 | 5-imidazolyl | H | H | H | N/A | 0 | 0 |
| HAPH-2 | 5-imidazolyl | H | H | NH₂ | N/A | 0 | 1 |
| HAPH-2 | 5-imidazolyl | H | H | NH₂ HCl | N/A | 0 | 1 |
| HCl Salt | | | | | | | |
| HAPH-2 | 5-imidazolyl | H | H | N₃ | CH₂—NHC(O) | 0 | 11 |
| Azide | | | | | | | |
| HAPH-2 | 5-imidazolyl | H | H | CΞC | CH₂—NHC(O) | 0 | 11 |
| Ethyne | | | | | | | |
| HAPH-2 | 5-imidazolyl | H | H | N₃ | N/A | 0 | 1 |
| Methylene | | | | | | | |
| Azide | | | | | | | |
| HAPH-3 | 5-imidazolyl | H | H | OH | N/A | 0 | 1 |
| HAPH-4 | 5-imidazolyl | H | H | COOH | N/A | 0 | 2 |
| HAPH-5 | 5-imidazolyl | H | H | COOH | N/A | 0 | 3 |
| HAPH-6 | 5-imidazolyl | H | H | NH₂ | N/A | 0 | 2 |
| HAPH-7 | 5-imidazolyl | H | H | NH₂ | N/A | 0 | 3 |
| HAPH-8 | 5-imidazolyl | H | H | OH | N/A | 0 | 2 |
| HAPH-9 | 5-imidazolyl | H | H | OH | N/A | 0 | 3 |
| HAPH-10 | 5-imidazolyl | H | H | NH₂ | O | 1 | 2 |
| HAPH-11 | 5-imidazolyl | H | H | COOH | O | 1 | 2 |
| HAPH-12 | 5-imidazolyl | H | H | OH | O | 1 | 2 |
| HAPH-13 | 5-imidazolyl | H | H | COOH | N/A | 0 | 1 |
| AMPHIS-1N | —NH₂ | —C(O)OCH₃ | H | NH₂ | N/A | 0 | 1 |
| AMPHIS-1O | —NH₂ | —C(O)OCH₃ | H | OH | N/A | 0 | 1 |
| AMPHIS-1A | —NH₂ | —C(O)OCH₃ | H | COOH | N/A | 0 | 1 |
| PYML-1N | NH₂ | —C(O)OH | —C(O)NH₂ | NH₂ | N/A | 0 | 1 |
| PYML-1O | NH₂ | —C(O)OH | —C(O)NH₂ | OH | N/A | 0 | 1 |
| PYML-1A | NH₂ | —C(O)OH | —C(O)NH₂ | COOH | N/A | 0 | 1 |
| SAPH-1N | —SMe | H | H | NH₂ | N/A | 0 | 1 |
| SAPH-1O | —SMe | H | H | OH | N/A | 0 | 1 |
| SAPH-1A | —SMe | H | H | COOH | N/A | 0 | 1 |
| SAPH-2N | —SCH₂C₆H₄OMe | H | H | NH₂ | N/A | 0 | 1 |
| SAPH-2O | —SCH₂C₆H₄OMe | H | H | OH | N/A | 0 | 1 |
| SAPH-2A | —SCH₂C₆H₄OMe | H | H | COOH | N/A | 0 | 1 |
| SAPH-3N | —SH | H | H | NH₂ | N/A | 0 | 1 |
| SAPH-3O | —SH | H | H | OH | N/A | 0 | 1 |
| SAPH-3A | —SH | H | H | COOH | N/A | 0 | 1 |

11. A composition comprising an amount of the compound of claim 1, and a carrier.

12. The composition of claim 11, wherein the amount is a therapeutically effective amount, and the carrier is a pharmaceutically acceptable carrier.

13. The composition of claim 11, wherein the amount is a diagnostic effective amount, and the carrier is a diagnostically acceptable carrier.

14. The composition of claim 11, wherein the amount is a theranostic effective amount, and the carrier is a theranostic acceptable carrier.

15. (canceled)

16. (canceled)

17. The composition of claim 1, wherein the carrier is suitable for parenteral delivery comprising intravenous injection, intratumor injection, subcutaneous injection, intramuscular injection, or intrathecal injection.

18. (canceled)

19. (canceled)

20. A method of inducing DNA scission or cleavage in a subject in need thereof by administering an effective amount of the compound of claim 1.

21. (canceled)

22. The method of claim 19, wherein the subject is having or suspected of having a cancer, a tumor, a blood pool, a thrombus, an inflammation, a hypoxia, a myocardial abnormality, a brain abnormality, or a disorder related with oxygen activity abnormality, dysfunction, deficiency, or disruption.

23. (canceled)

24. (canceled)

25. A tumor-imaging method by administering the metal chelate of claim 6 to a subject in need thereof, wherein the metal chelate is a fluorescent metal chelate or a radioisotope metal chelate.

26. (canceled)

27. The method of claim 25, wherein the radioisotope metal chelate is used in PET, MRI, and/or CT.

28. The method of claim 27, wherein the radioisotope comprises ⁴³Sc, ⁴⁴Sc, ⁴⁶Sc, ⁴⁷Sc, ⁴⁸Sc, ⁵⁵Co, ⁶⁰Cu, ⁶¹Cu, ⁶²Cu, ⁶⁴Cu, ⁶⁷Cu, ¹⁸F, ⁶⁶Ga, ⁶⁷Ga, ⁶⁸Ga, ¹⁸⁸Re, ¹¹¹In, ¹¹³In, ⁹⁰Y, ⁸⁶Y or ^{99m}Tc.

29. The method of claim 25, wherein the subject has or is suspected of having a cancer, a tumor a blood pool, a thrombus, an inflammation, a hypoxia, a myocardial abnormality, a brain abnormality, or a disorder related with oxygen activity abnormality, dysfunction, deficiency, disruption.

30-33. (canceled)
